(19) United States
(12) Patent Application Publication
(10) Pub. No.: US 2022/0372501 A1
(43) Pub. Date: Nov. 24, 2022

(54) PRODUCTION OF OLIGOSACCHARIDES

(71) Applicant: Ginkgo Bioworks, Inc., Boston, MA (US)

(72) Inventors: Sudeep Agarwala, Boston, MA (US); Michael G. Napolitano, Boston, MA (US)

(73) Assignee: Ginkgo Bioworks, Inc., Boston, MA (US)

(21) Appl. No.: 17/763,152

(22) PCT Filed: Sep. 24, 2020
(86) PCT No.: PCT/US2020/052390

§ 371 Date: Mar. 23, 2022

Related U.S. Application Data
(60) Provisional application No. 62/905,246, filed on Sep. 24, 2019.

(51) Int. Cl. C12N 15/81; C12N 9/10; C12P 19/18

(52) U.S. Cl. CPC C12N 15/81; C12N 9/1055; C12Y 204/01099; C12Y 204/011; C12P 19/18

(57) ABSTRACT

The disclosure relates to methods and compositions for the production of fructans using sucrose:sucrose 1-fructosyl-transferase (1-SST), fructan:fructan 1-fructosyltransferase (1-FFT), and/or sucrose fructan-6-fructosyltransferase (6-SFT) enzymes.

### CROSS-REFERENCE TO RELATED APPLICATIONS

This application claims the benefit under 35 U.S.C. § 119(e) of U.S. Provisional Application Ser. No. 62/905,246, filed Sep. 24, 2019, entitled “PRODUCTION OF OLIGOSACCHARIDES,” the disclosure of which is incorporated by reference herein in its entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Sep. 23, 2020, is named G091970034WO00-SEQ-FL and is 276 kilobytes in size.

### FIELD OF INVENTION The disclosure relates to enzymes, nucleic acids, and cells useful for the conversion of sucrose to fructans.

### BACKGROUND

Polyfructans are oligosaccharides that comprise fructose monomers. These oligosaccharides generally further comprise glucose. Polyfructans have numerous uses including as prebiotics, fat replacers, sugar replacers, texture modifiers, and in industrial processes. Polyfructans may comprise β(2,6) linkages and/or β(2,1) linkages, with the type of polyfructan depending on the linkage position of the fructose residues. For example, graminans are complex mixtures of branched polyfructan oligosaccharides with β(2,1)-linked-D-fructosyl backbone and β(2,6)-linked-D-fructosyl side chains with different degrees of polymerization. Three distinct classes of enzymes can be used to produce polyfructans: sucrose:sucrose 1-fructosyltransferase (1-SST) enzymes, which generate branched polyfructans by introduction of β(2,1) linkages in saccharides; fructan:fructan 1-fructosyltransferase (1-FFT) enzymes, which promote polymerization of fructose monomers on saccharides though the formation of β(2,1) linkages; and sucrose:fructan-6-fructosyltransferase (6-SFT) enzymes, which catalyze the addition of fructose monomers through β(2,6) linkages to produce polyfructans.

### SUMMARY

This disclosure relates, at least in part, to generation of engineered cells containing enzymes for producing polyfructan oligosaccharides, for example, by converting sucrose to polyfructans. These engineered cells are useful for producing complex and branched polyfructans.

Aspects of the disclosure relate to host cells that comprise one or more heterologous polynucleotides encoding: a sucrose:sucrose 1-fructosyltransferase (1-SST) enzyme; a fructan:fructan 1-fructosyltransferase (1-FFT); and a sucrose:fructan-6-fructosyltransferase (6-SFT) enzyme.

In some embodiments, the 1-SST enzyme comprises an amino acid sequence that is at least 90% identical to SEQ ID NO: 1 or SEQ ID NO: 24.

In some embodiments, the 1-FFT enzyme comprises an amino acid sequence that is at least 90% identical to SEQ ID NO: 7 or SEQ ID NO: 31.

In some embodiments, the 6-SFT enzyme comprises an amino acid sequence that is at least 90% identical to SEQ ID NO: 13 or SEQ ID NO: 38.

In some embodiments, a host cell comprises one or more heterologous polynucleotides encoding two or more of a 1-SST enzyme; a 1-FFT enzyme; and a 6-SFT enzyme.

In some embodiments, a host cell comprises one or more heterologous polynucleotides encoding a 1-SST enzyme; a 1-FFT enzyme; and a 6-SFT enzyme.

In some embodiments, at least two of the 1-SST enzyme, the 1-FFT enzyme, and the 6-SFT enzyme are expressed on the same heterologous polynucleotide.

In some embodiments, the host cell is a plant cell, an algal cell, a yeast cell, a bacterial cell, or an animal cell.

In some embodiments, the yeast cell is a Saccharomyces cell, a Yarrowia cell or a Pichia cell. In some embodiments, the host cell is a Pichia pastoris cell.

In some embodiments, the 1-SST enzyme comprises the amino acid sequence of SEQ ID NO: 1 or SEQ ID NO: 24.

In some embodiments, the 1-FFT enzyme comprises the amino acid sequence of SEQ ID NO: 7 or SEQ ID NO: 31.

In some embodiments, the 6-SFT enzyme comprises the amino acid sequence of SEQ ID NO: 13 or SEQ ID NO: 38.

In some embodiments, one or more of the 1-SST enzyme, the 1-FFT enzyme, and the 6-SFT enzyme is secreted from the host cell.

Further aspects of the disclosure provide methods comprising culturing any of the host cells disclosed herein in this application.

In some embodiments, the methods further comprise purifying one or more of the 1-SST enzyme, 1-FFT enzyme, and 6-SFT enzyme from the host cell.

Further aspects of the disclosure provide methods of producing a fructan. In some embodiments, the method comprises contacting sucrose with one or more of (a) a 1-SST enzyme comprising an amino acid sequence that is at least 90% identical to SEQ ID NO: 1 or SEQ ID NO: 24; (b) a 1-FFT enzyme comprising an amino acid sequence that is at least 90% identical to SEQ ID NO: 7 or SEQ ID NO: 31; and (c) a 6-SFT enzyme comprising an amino acid sequence that is at least 90% identical to SEQ ID NO: 13 or SEQ ID NO: 38.

In some embodiments, the sucrose is contacted with two or more of a 1-SST enzyme, a 1-FFT enzyme, and a 6-SFT enzyme.

In some embodiments, the sucrose is contacted with a 1-SST enzyme, a 1-FFT enzyme, and a 6-SFT enzyme.

In some embodiments, the fructan comprises a β(2,1) linkage, a β(2,6) linkage, or a combination thereof.

In some embodiments, the fructan is a kestose, an inulin and/or a graminan.

In some embodiments, the fructan has a degree of polymerization of at least 3.

In some embodiments, the method further comprises purifying the fructan.

In some embodiments, the 1-SST enzyme, 1-FFT enzyme, and/or 6-SFT enzyme are secreted from one or more host cells.

In some embodiments, the one or more host cells are cultured in media containing sucrose, wherein the sucrose is contacted with the 1-SST enzyme, 1-FFT enzyme, and/or 6-SFT enzyme in the media.

In some embodiments, the fructan is purified from the media.

In some embodiments, the 1-SST enzyme, 1-FFT enzyme, and/or 6-SFT enzyme is a purified enzyme.

In some embodiments, the kestose is 6-kestose.

In some embodiments, the kestose is 1-kestose.

In some embodiments, the fructan comprises a levan.

Aspects of the disclosure provide methods of producing a fructan, comprising (a) contacting sucrose with a 1-SST enzyme to produce kestose; and (b) contacting the kestose with a 1-FFT enzyme and/or a 6-SFT enzyme to produce the fructan.

In some embodiments, the kestose produced in a) is purified and the purified kestose is contacted with the 1-FFT enzyme and/or 6-SFT enzyme in b).

In some embodiments, the method further comprises purifying the fructan produced in b).

In some embodiments, the 1-SST enzyme, 1-FFT enzyme, and/or 6-SFT enzyme is secreted from one or more host cells. In some embodiments, the one or more host cells is cultured in media containing sucrose, wherein the sucrose is contacted with the 1-SST enzyme in the media. In some embodiments, the 1-SST enzyme, 1-FFT enzyme, and/or 6-SFT enzyme is a purified enzyme. In some embodiments, the fructan produced in b) is an inulin. In some embodiments, the fructan produced in b) is a branched inulin. In some embodiments, the fructan produced in b) is a graminan.

Aspects of the disclosure provide host cells that comprise one or more heterologous polynucleotides encoding one or more of (a) a 1-SST enzyme comprising an amino acid sequence that is at least 90% identical to a sequence selected from SEQ ID NOs: 1-4 and 24-28; (b) a 1-FFT enzyme comprising an amino acid sequence that is at least 90% identical to a sequence selected from SEQ ID NOs: 7-10 and 31-35; and (c) a 6-SFT enzyme comprising an amino acid sequence that is at least 90% identical to a sequence selected from SEQ ID NO: 13-21 and 38-52.

Aspects of the disclosure provide methods of producing a fructan, comprising contacting sucrose with one or more of: (a) a 1-SST enzyme comprising an amino acid sequence that is at least 90% identical to a sequence selected from SEQ ID NOs: 1-4 and 24-28; (b) a 1-FFT enzyme comprising an amino acid sequence that is at least 90% identical to a sequence selected from SEQ ID NOs: 7-10 and 31-35; and (c) a 6-SFT enzyme comprising an amino acid sequence that is at least 90% identical to a sequence selected from SEQ ID NOs: 13-21 and 38-52.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used in this disclosure is for the purpose of description and should not be regarded as limiting. The use of “including,” “comprising,” or “having,” “containing,” “involving,” and variations thereof, is meant to encompass the items listed thereafter and equivalents thereof as well as additional items.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 depicts schematics showing chemical structures of selected fructans (inulins, levans, and graminans).

FIG. 2 depicts a schematic showing an example of biosynthetic conversion and relevant enzymes involved in the production of fructans in Agave tequiliana.

FIGS. 3A-3B depict graphs showing data from screening of a library of enzymes. FIG. 3A shows a graph displaying individual enzymes and the resultant products (β(2,6) fructans (labeled ‘2→6’ on y-axis) or β(2,1) fructans (labeled ‘kestose’ on x-axis)) formed by incubation with sucrose. Based on product formation, individual enzymes were classified as: inactive; having invertase activity; having kestose transferase (1-SST) activity; or having β(2,6) branching (6-SFT) activity. FIG. 3B shows a graph displaying individual enzymes and the resultant products (β(2,1) inulins (labeled ‘Nystose’ on y-axis) or β(2,1) fructans (labeled ‘kestose’ on x-axis)) formed by incubation with kestose. Based on product formation, individual enzymes were classified as: inactive; having kestase activity; or having 1-FFT activity. All reaction products in FIGS. 3A-3B were analyzed by HPLC and quantified using peak integration.

FIG. 4 depicts schematics showing representative HPLC-RID traces of fructans. An example of an enzymatic bioconversion reaction (individual enzyme incubated with sucrose) is shown in the top panel. An example of a preparation of commercially-available standards of nystose (A), 1-kestose (B), sucrose (C), glucose (D), and fructose (E) is shown in the bottom panel.

FIG. 5 depicts a schematic showing synthesis of branched inulins. Starting from sucrose (dimer of glucose and fructose), kestose (comprising β(2,1) linkage) is enzymatically formed using 1-SST activity. 1-FFT activity catalyzes formation of a linear inulin, which can be reacted with an enzyme having 6-SFT activity to provide β2,6 branched inulins. (G=glucose; F=fructose.)

FIGS. 6A-6D show confirmation of branched inulin formation by bioconversion. FIG. 6A shows an HPLC-RID trace of a bioconversion reaction showing that branched inulins have been produced and can be distinguished from starting material (sucrose) and by-products (glucose). FIG. 6B shows a schematic depicting fragmentation products that are generated when branched inulins are subjected to analysis by GC/MS. These fragmentation products provide a unique mass spectroscopy signature that indicates presence of β2,6 branching. FIG. 6C shows an example of GC/MS spectral analysis of: a bioconversion sample; linear sugars (Chicory; Nicie); and a known branched sugar (‘Test Ground’). FIG. 6D is a magnification of the GC/MS analysis in FIG. 6C between 28.0-29.6 min.

FIG. 7 is a non-limiting example of sequence identity analysis of SEQ ID NOs: 2-4, 6, 8-10, 12, 14-21, and 63. The percent sequence identity between indicated SEQ ID NOs is shown. SEQ ID NO: 6 is Festuca arundinacea 1-SST. SEQ ID NO: 12 is Echinops ritro 1-FFT. SEQ ID NO: 63 corresponds to residues 60 through 623 of Phleum pratense 6-SFT (SEQ ID NO: 23). Multiple Sequence Comparison by Log-Expectation (MUSCLE) was used for the sequence identity analysis.

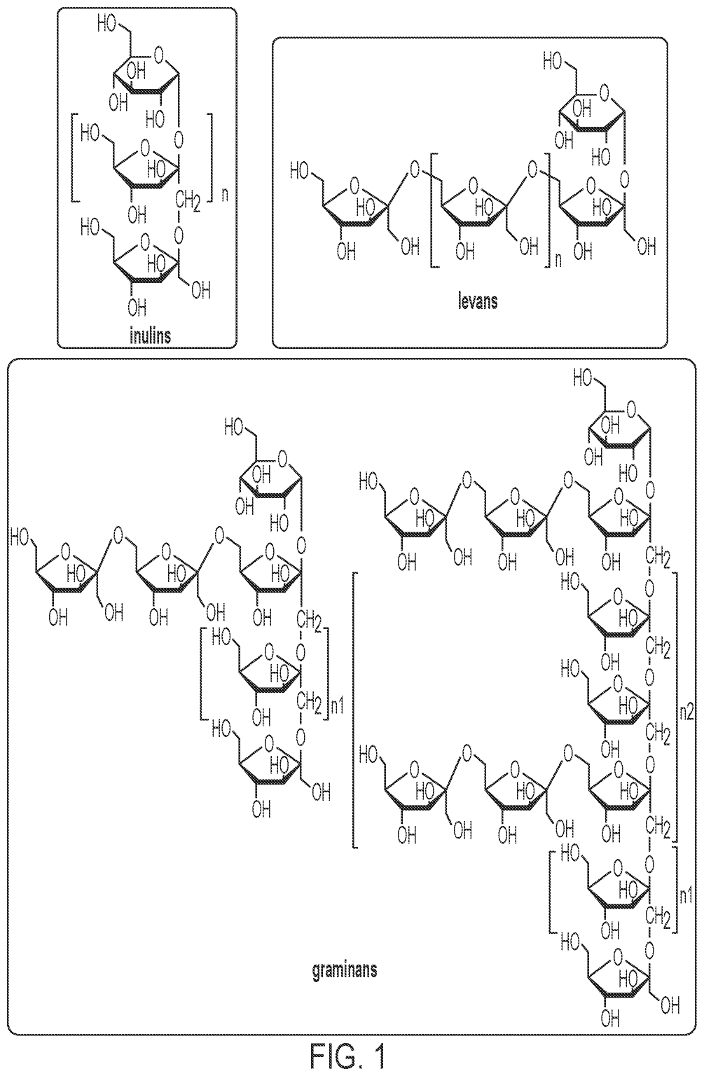

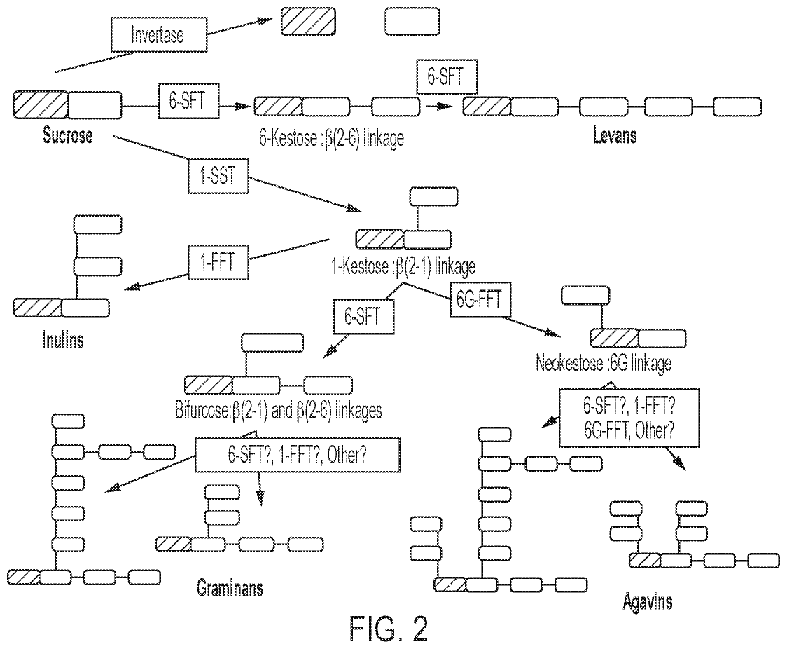

### DETAILED DESCRIPTION OF THE INVENTION

The disclosure provides, in some aspects, cells and enzymes that are engineered for production of polyfructans from sucrose. These enzymes include 1-SST enzymes, 1-FFT enzymes, and 6-SFT enzymes. Enzymes disclosed in this application and host cells comprising such enzymes, may be used to promote production of fructans, including branched fructans, such as branched inulins. In some embodiments, a fructan comprises a β(2,1) linkage, a β-(2,6)-linkage, or a combination thereof.

Fructans

As used in this application, a “fructan,” which may also be referred to as a “polyfructan” or a “fructooligosaccharide,” refers to an oligosaccharide that comprises fructose monomers. Fructans generally further comprise glucose. In some embodiments, a fructan comprises at least one β(2,1) linkage, at least one β(2,6) linkage, or a combination thereof. In some embodiments, a fructan is a kestose (e.g., 1-kestose or 6-kestose), an inulin and/or a graminan. In some embodiments, a fructan has a degree of polymerization (DP) of at least 3 (e.g., at least 3, at least 4, at least 5, at least 6), wherein the degree of polymerization refers to the total number of monosaccharide units (e.g., fructose units) in a fructan or the average number of monosaccharide units in a mixture of fructans. In some embodiments, a fructan comprises a levan (e.g., a linear levan or a branched levan, e.g., comprising at least one β(2,1) linkage and/or at least one β(2,6) linkage). In some embodiments, a fructan is an inulin. In some embodiments, an inulin is a linear inulin or a branched inulin (e.g., comprising at least one β(2,1) linkage and/or at least one β(2,6) linkage). In some embodiments, a fructan is a graminan.

Formula 1 is an example of a fructan comprising a β(2,1) linkage:

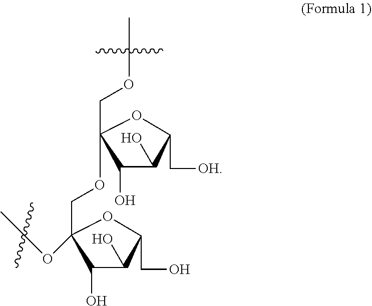

Formula 2 is an example of a fructan comprising a β(2,6) linkage:

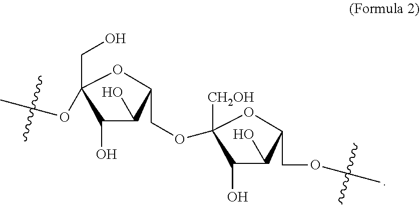

Formula 3 shows 1-kestose:

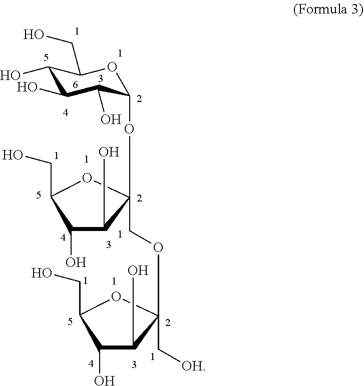

Formula 4 shows 6-kestose:

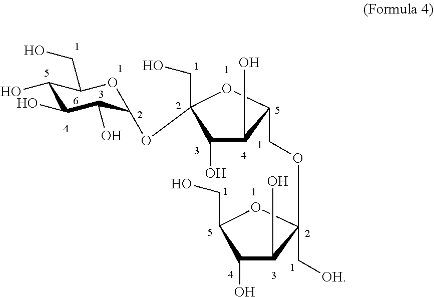

Formula 5 shows nystose:

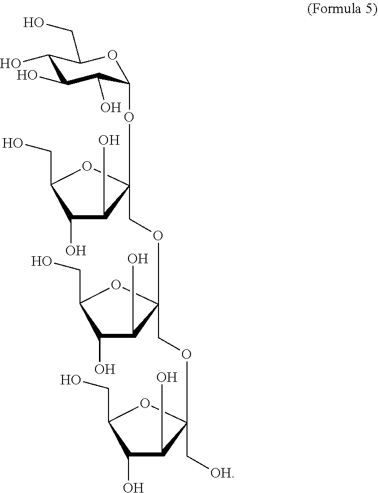

Formula 6 shows an inulin, in which n is any integer.

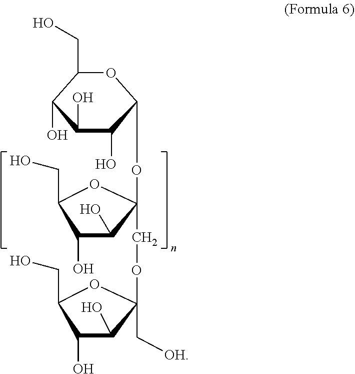

Formula 7 shows an example of a graminan, in which n1 is any integer.

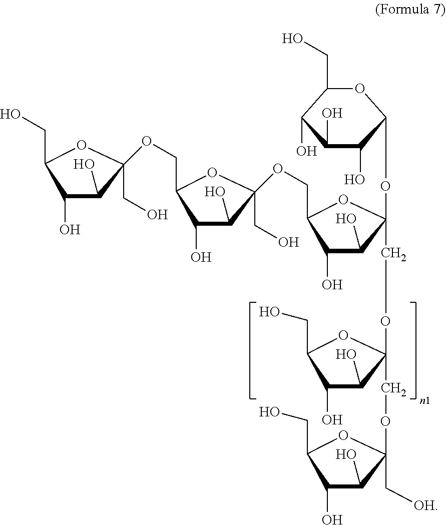

Formula 8 shows an example of a graminan, in which n1 and n2 independently may be any integer.

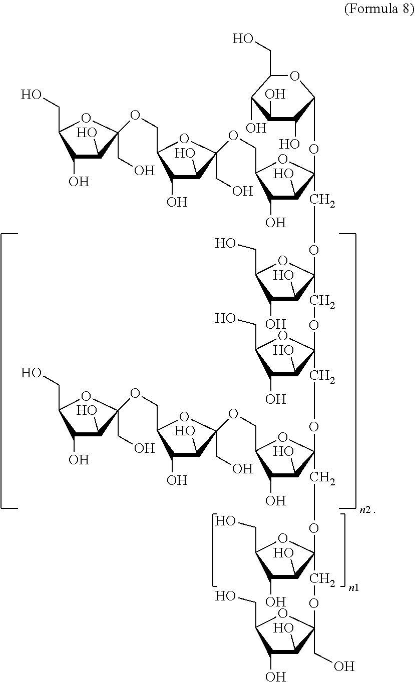

As one of ordinary skill in the art would appreciate, any of the fructans produced using the methods described in this application may have numerous applications, including industrial uses. As a non-limiting example, long chain fructans (e.g., levans) may be used in fermentation processes and in the production of vinegar. See also, e.g., Niness, J Nutr. 1999 Jul; 129(7 Suppl):1402S-6S; Kolida et al., Br J Nutr. 2002; Koga et al., Pediatr Res. 2016 Dec; 80(6):844-851; Roberfroid, J Nutr. 2007 Nov; 137(11 Suppl):24935-25025; Suzuki et al., Bioscience Microflora Vol. 25(3), 109-116, 2006; Lopez and Urias-Silvas, Recent Advances in Fructooligosaccharides Research (pp. 297-310), 2007; and Vijn and Smeekens, Plant Physiology, June 1999, Vol. 120, pp. 351-359.

Sucrose:sucrose 1-fructosyltransferase (1-SST)

As used in this application, “sucrose:sucrose 1-fructosyltransferase (1-SST)” refers to an enzyme that generates branched polyfructans by introduction of β(2,1) linkages in saccharides (e.g., formation of 1-kestose from sucrose). A 1-SST enzyme may use sucrose as a substrate. In some embodiments, 1-SST exhibits specificity for sucrose compared to other saccharides. In some embodiments, 1-SST produces 1-kestose from sucrose. In some embodiments, a 1-SST can use levan as a substrate to produce a branched levan with beta(2-6) linkages and beta(2-1) linkages.

A host cell described in this application can comprise a 1-SST enzyme and/or a heterologous polynucleotide encoding such an enzyme. In some embodiments, a host cell comprises a heterologous polynucleotide encoding a 1-SST enzyme comprising an amino acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NOs: 1-4, 6, and 24-28; a 1-SST enzyme in Table 2; or a 1-SST enzyme otherwise described in this application. In some embodiments, a host cell comprises a heterologous polynucleotide that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NOs: 5, 29-30, and 62; a polynucleotide encoding a 1-SST enzyme in Table 2; or a polynucleotide encoding a 1-SST enzyme otherwise described in this application.

In some embodiments, a host cell does not comprise a 1-SST derived from Festuca arundinacea. In some embodiments, a host cell does not comprise a 1-SST corresponding to SEQ ID NO: 6.

In some embodiments, a host cell that expresses a heterologous polynucleotide encoding a 1-SST enzyme may increase conversion of sucrose to 1-kestose, and/or increase introduction of β(2,1) linkages in oligosaccharides, by 0.5-fold, 1-fold, 1.5-fold, 2-fold, 2.5-fold, 3-fold, 3.5-fold, 4-fold, 4.5-fold, 5-fold, 5.5-fold, or 6-fold more (e.g., 2-fold to 6-fold more) relative to a control. In some embodiments, the control is a host cell that expresses a heterologous polynucleotide encoding SEQ ID NO: 6. In some embodiments, the control is a Pichia pastoris strain that expresses a heterologous polynucleotide encoding SEQ ID NO: 6, such as is described in and incorporated by reference from Lüscher, M. et. al., “Cloning and Functional Analysis of Sucrose:Sucrose 1-Fructosyltransferase from Tall Fescue,” Plant Physiology, 124:1217-1227 (2000).

In some embodiments, a host cell that expresses a heterologous polynucleotide encoding a 1-SST enzyme may exhibit at least 0.5-fold, 1-fold, 1.5-fold, 2-fold, 2.5-fold, 3-fold, 3.5-fold, 4-fold, 4.5-fold, 5-fold, 5.5-fold, or 6-fold more (e.g., 2-fold to 6-fold more) more activity in the presence of sucrose relative to other saccharides. In some embodiments, activity corresponds to conversion of sucrose to 1-kestose, and/or increase introduction of β(2,1) linkages in oligosaccharides.

In some embodiments, a 1-SST comprises a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to any one of SEQ ID NOs: 1-4, 6, and 24-28.

Fructan:fructan 1-fructosyltransferase (1-FFT)

As used in this application, “fructan:fructan 1-fructosyltransferase (1-FFT)” refers to an enzyme that catalyzes the conversion of oligosaccharides comprising β(2,1) linkages (e.g., 1-kestose) into longer polymer chains of oligosaccharides (e.g., conversion of 1-kestose to inulins). A 1-FFT enzyme may use 1-kestose, sucrose, and/or fructose as a substrate. In some embodiments, a 1-FFT enzyme can use bifurcose or neokestose as a substrate. In some embodiments, 1-FFT produces inulins (e.g., branched inulins) from 1-kestose.

A host cell described in this application can comprise a 1-FFT enzyme and/or a heterologous polynucleotide encoding such an enzyme. In some embodiments, a host cell comprises a heterologous polynucleotide encoding a 1-FFT enzyme comprising an amino acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 7-10, 12, and 31-35; a 1-FFT enzyme in Table 2; or a 1-FFT enzyme otherwise described in this application. In some embodiments, a host cell comprises a heterologous polynucleotide that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 11, 36, and 37; a polynucleotide encoding a 1-FFT enzyme in Table 2; or a polynucleotide encoding a 1-FFT enzyme otherwise described in this application.

In some embodiments, a host cell does not comprise a 1-FFT enzyme derived from Echinops ritro. In some embodiments, a host cell does not comprise a 1-FFT enzyme corresponding to SEQ ID NO: 12.

In some embodiments, a host cell that expresses a heterologous polynucleotide encoding a 1-FFT enzyme may increase conversion of 1-kestose to inulins, and/or increase conversion of oligosaccharides comprising β(2,1) linkages into longer polymer chains of oligosaccharides, by 0.5-fold, 1-fold, 1.5-fold, 2-fold, 2.5-fold, 3-fold, 3.5-fold, 4-fold, 4.5-fold, 5-fold, 5.5-fold, or 6-fold more (e.g., 2-fold to 6-fold more) relative to a control. In some embodiments, a control is a host cell that expresses a heterologous polynucleotide encoding SEQ ID NO: 12. In some embodiments, the control is a Pichia pastoris strain that expresses a heterologous polynucleotide encoding SEQ ID NO: 12, such as is described in and incorporated by reference from Van den Ende, W. et al., “Cloning and Functional Analysis of a High DP Fructan:Fructan 1-Fructosyl transferase from Echinops ritro (Asteraceae): Comparison of the native and recombinant enzymes,” Journal of Experimental Botany, 57(4):775-789 (2006).

In some embodiments, a 1-FFT enzyme comprises a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to any one of SEQ ID NO: 7-10, 12, and 31-35.

Sucrose:fructan-6-fructosyltransferase (6-SFT)

As used in this application “sucrose:fructan-6-fructosyltransferase (6-SFT)” refers to an enzyme that generates fructans by introducing β(2,6) linkages in saccharides (e.g., production of 6-kestose from sucrose) or generates more complex fructans by introducing β(2,6) linkages in precursor fructans (e.g., production of bifurcose from 1-kestose). A 6-SFT may use sucrose, 6-kestose, 1-kestose, bifurcose, and/or neokestose as a substrate. In some embodiments, 6-SFT produces 6-kestose from sucrose. In some embodiments, 6-SFT produces bifurcose from 1-kestose. In some embodiments, 6-SFT produces graminans from bifurcose.

A host cell described in this application can comprise a 6-SFT enzyme and/or a heterologous polynucleotide encoding such an enzyme. In some embodiments, a host cell comprises a heterologous polynucleotide encoding a 6-SFT enzyme comprising an amino acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NO: 13-21, 23, and 38-52; a 6-SFT enzyme in Table 2; or a 6-SFT enzyme otherwise described in this application. In some embodiments, a host cell comprises a heterologous polynucleotide that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical, or is 100% identical, including all values in between, to any of: SEQ ID NOs: 22 and 53-59; a polynucleotide encoding a 6-SFT enzyme in Table 2; or a polynucleotide encoding a 6-SFT enzyme otherwise described in this application.

In some embodiments, the host cell does not comprise a 6-SFT enzyme derived from Phleum pratense. In some embodiments, the host cell does not comprise a 6-SFT enzyme corresponding to SEQ ID NO: 23. In some embodiments, the host cell does not comprise a 6-SFT enzyme corresponding to SEQ ID NO: 63.

In some embodiments, a host cell that expresses a heterologous polynucleotide encoding an 6-SFT enzyme may increase conversion of sucrose to 1-kestose, increase conversion of 1-kestose to bifurcose, increase conversion of bifurcose to graminans, and/or increase introduction of β(2,6) linkages into fructans by 0.5-fold, 1-fold, 1.5-fold, 2-fold, 2.5-fold, 3-fold, 3.5-fold, 4-fold, 4.5-fold, 5-fold, 5.5-fold, or 6-fold more (e.g., 2-fold to 6-fold more) relative to a control. In some embodiments, a control is a host cell that expresses a heterologous polynucleotide encoding SEQ ID NO: 23. In some embodiments, the control is a Pichia pastoris strain that expresses a heterologous polynucleotide encoding SEQ ID NO: 23, such as is described in and incorporated by reference from Tamura, K. I., et al. “Cloning and Functional Analysis of a Fructosyltransferase cDNA for Synthesis of Highly Polymerized Levans in Timothy (Phleum pratense L.)” Journal of Experimental Botany, 60(3), 893-905 (2009). In some embodiments, a control is a host cell that expresses a heterologous polynucleotide encoding SEQ ID NO: 63. In some embodiments, the control is a Pichia pastoris strain that expresses a heterologous polynucleotide encoding SEQ ID NO: 63.

In some embodiments, an 6-SFT comprises a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to any one of SEQ ID NOs:13-21, 23, and 38-52.

Variants

Variants of enzymes and proteins described in this application (e.g., 1-SST, 1-FFT, or 6-SFT), including variants to nucleic acid and amino acid sequences, are also encompassed by the present disclosure. A variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a reference sequence, including all values in between.

Unless otherwise noted, the term “sequence identity,” as known in the art, refers to a relationship between the sequences of two polypeptides or polynucleotides, as determined by sequence comparison (alignment). In some embodiments, sequence identity is determined across the entire length of a sequence, such as a reference sequence, while in other embodiments, sequence identity is determined over a region of a sequence. In some embodiments, sequence identity is determined over a region (e.g., a stretch of amino acids or nucleic acids, e.g., the sequence spanning an active site) of a sequence (e.g., 1-SST, 1- FFT, or 6-SFT sequence). For example, in some embodiments, sequence identity is determined over a region corresponding to at least 30%, at least 40%, at least 50%, at least 60%, at least 70%, at least 80%, at least 90%, at least 95%, or over 100% of the length of the reference sequence.

Identity measures the percent of identical matches between the smaller of two or more sequences with gap alignments (if any) addressed by a particular mathematical model, algorithm, or computer program.

Identity of related polypeptides or nucleic acid sequences can be readily calculated by any of the methods known to one of ordinary skill in the art. The percent identity of two sequences (e.g., nucleic acid or amino acid sequences) may, for example, be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST® protein searches can be performed, for example, with the XBLAST program, score=50, wordlength=3 to obtain amino acid sequences homologous to the proteins described in this application. Where gaps exist between two sequences, Gapped BLAST® can be utilized, for example, as described in Altschul et al., Nucleic Acids Res. 25(17):3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

Another local alignment technique which may be used, for example, is based on the Smith-Waterman algorithm (Smith, T. F. & Waterman, M. S. (1981) “Identification of common molecular subsequences.” J. Mol. Biol. 147:195-197). A general global alignment technique which may be used, for example, is the Needleman-Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two polypeptides is determined by aligning the two amino acid sequences, calculating the number of identical amino acids, and dividing by the length of one of the amino acid sequences. In some embodiments, the identity of two nucleic acids is determined by aligning the two nucleotide sequences and calculating the number of identical nucleotide and dividing by the length of one of the nucleic acids.

For multiple sequence alignments, computer programs including Clustal Omega (Sievers et al., Mol Syst Biol. 2011 Oct 11;7:539) may be used.

In preferred embodiments, a sequence, including a nucleic acid or amino acid sequence, is found to have a specified percent identity to a reference sequence, such as a sequence disclosed in this application and/or recited in the claims when sequence identity is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993 (e.g., BLAST®, NBLAST®, XBLAST® or Gapped BLAST® programs, using default parameters of the respective programs).

In some embodiments, a sequence, including a nucleic acid or amino acid sequence, is found to have a specified percent identity to a reference sequence, such as a sequence disclosed in this application and/or recited in the claims when sequence identity is determined using the Smith-Waterman algorithm (Smith, T. F. & Waterman, M. S. (1981) “Identification of common molecular subsequences.” J. Mol. Biol. 147:195-197) or the Needleman-Wunsch algorithm (Needleman, S. B. & Wunsch, C. D. (1970) “A general method applicable to the search for similarities in the amino acid sequences of two proteins.” J. Mol. Biol. 48:443-453) using default parameters.

In some embodiments, a sequence, including a nucleic acid or amino acid sequence, is found to have a specified percent identity to a reference sequence, such as a sequence disclosed in this application and/or recited in the claims when sequence identity is determined using a Fast Optimal Global Sequence Alignment Algorithm (FOGSAA) using default parameters.

In some embodiments, a sequence, including a nucleic acid or amino acid sequence, is found to have a specified percent identity to a reference sequence, such as a sequence disclosed in this application and/or recited in the claims when sequence identity is determined using Clustal Omega (Sievers et al., Mol Syst Biol. 2011 Oct 11;7:539) using default parameters.

As used in this application, a residue (such as a nucleic acid residue or an amino acid residue) in sequence “X” is referred to as corresponding to a position or residue (such as a nucleic acid residue or an amino acid residue) “n” in a different sequence “Y” when the residue in sequence “X” is at the counterpart position of “n” in sequence “Y” when sequences X and Y are aligned using amino acid sequence alignment tools known in the art.

Variant sequences may be homologous sequences. As used in this application, homologous sequences are sequences, including nucleic acid or amino acid sequences, that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity, including all values in between). Homologous sequences include but are not limited to paralogous sequences, orthologous sequences, or sequences arising from convergent evolution. Paralogous sequences arise from duplication of a gene within a genome of a species, while orthologous sequences diverge after a speciation event. Two different species may have evolved independently but may each comprise a sequence that shares a certain percent identity with a sequence from the other species as a result of convergent evolution.

In some embodiments, a polypeptide variant, such as a 1-SST, 1-FFT, or 6-SFT enzyme variant, comprises a domain that shares a secondary structure (e.g., alpha helix, beta sheet) with a reference polypeptide (e.g., a reference 1-SST, 1-FFT, or 6-SFT enzyme). In some embodiments, a polypeptide variant, such as a 1-SST, 1-FFT, or 6-SFT enzyme variant, shares a tertiary structure with a reference polypeptide (e.g., a reference 1-SST, 1-FFT, or 6-SFT enzyme). As a non-limiting example, a variant polypeptide (e.g., 1-SST, 1-FFT, or 6-SFT enzyme variant) may have low primary sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity) compared to a reference polypeptide, but share one or more secondary structures (e.g., including but not limited to loops, alpha helices, or beta sheets), or have the same or similar tertiary structure as a reference polypeptide. For example, a loop may be located between a beta sheet and an alpha helix, between two alpha helices, or between two beta sheets. Homology modeling may be used to compare two or more tertiary structures.

Mutations can be made in a nucleotide sequence by a variety of methods known to one of ordinary skill in the art. For example, mutations can be made by PCR-directed mutation, site-directed mutagenesis according to the method of Kunkel (Kunkel, Proc. Nat. Acad. Sci. U.S.A. 82: 488-492, 1985), by chemical synthesis of a gene encoding a polypeptide, by gene editing, or by insertions, such as insertion of a tag (e.g., a HIS tag or a GFP tag). Mutations can include, for example, substitutions, deletions, and translocations, generated by any method known in the art. Methods for producing mutations may be found in references such as Molecular Cloning: A Laboratory Manual, J. Sambrook, et al., eds., Fourth Edition, Cold Spring Harbor Laboratory Press, Cold Spring Harbor, N.Y., 2012, or Current Protocols in Molecular Biology, F. M. Ausubel, et al., eds., John Wiley & Sons, Inc., New York, 2010.

In some embodiments, methods for producing variants include circular permutation (Yu and Lutz, Trends Biotechnol. 2011 Jan;29(1):18-25). In circular permutation, the linear primary sequence of a polypeptide can be circularized (e.g., by joining the N-terminal and C-terminal ends of the sequence) and the polypeptide can be severed (“broken”) at a different location. Thus, the linear primary sequence of the new polypeptide may have low sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less or less than 5%, including all values in between) as determined by linear sequence alignment methods (e.g., Clustal Omega or BLAST). Topological analysis of the two proteins, however, may reveal that the tertiary structure of the two polypeptides is similar or dissimilar. Without being bound by a particular theory, a variant polypeptide created through circular permutation of a reference polypeptide and with a similar tertiary structure as the reference polypeptide can share similar functional characteristics (e.g., enzymatic activity, enzyme kinetics, substrate specificity or product specificity). In some instances, circular permutation may alter the secondary structure, tertiary structure or quaternary structure and produce an enzyme with different functional characteristics (e.g., increased or decreased enzymatic activity, different substrate specificity, or different product specificity). See, e.g., Yu and Lutz, Trends Biotechnol. 2011 Jan;29(1):18-25.

It should be appreciated that in a protein that has undergone circular permutation, the linear amino acid sequence of the protein would differ from a reference protein that has not undergone circular permutation. However, one of ordinary skill in the art would be able to readily determine which residues in the protein that has undergone circular permutation correspond to residues in the reference protein that has not undergone circular permutation by, for example, aligning the sequences and detecting conserved motifs, and/or by comparing the structures or predicted structures of the proteins, e.g., by homology modeling.

In some embodiments, an algorithm that determines the percent identity between a sequence of interest and a reference sequence described in this application accounts for the presence of circular permutation between the sequences. The presence of circular permutation may be detected using any method known in the art, including, for example, RASPODOM (Weiner et al., Bioinformatics. 2005 Apr 1;21(7):932-7). In some embodiments, the presence of circulation permutation is corrected for (e.g., the domains in at least one sequence are rearranged) prior to calculation of the percent identity between a sequence of interest and a sequence described in this application. The claims of this application should be understood to encompass sequences for which percent identity to a reference sequence is calculated after taking into account potential circular permutation of the sequence.

Functional variants of the recombinant 1-SST, 1-FFT, or 6-SFT enzyme disclosed in this application are also encompassed by the present disclosure. For example, functional variants may bind one or more of the same substrates or produce one or more of the same products. Functional variants may be identified using any method known in the art. For example, the algorithm of Karlin and Altschul Proc. Natl. Acad. Sci. USA 87:2264-68, 1990 described above may be used to identify homologous proteins with known functions.

Putative functional variants may also be identified by searching for polypeptides with functionally annotated domains. Databases including Pfam (Sonnhammer et al., Proteins. 1997 Jul;28(3):405-20) may be used to identify polypeptides with a particular domain.

Homology modeling may also be used to identify amino acid residues that are amenable to mutation without affecting function. A non-limiting example of such a method may include use of position-specific scoring matrix (PSSM) and an energy minimization protocol.

Position-specific scoring matrix (PSSM) uses a position weight matrix to identify consensus sequences (e.g., motifs). PSSM can be conducted on nucleic acid or amino acid sequences. The method uses aligned sequences and takes into account the observed frequency of a particular residue (e.g., an amino acid or a nucleotide) at a particular position and the number of sequences analyzed. See, e.g., Stormo et al., Nucleic Acids Res. 1982 May 11;10(9):2997-3011. The likelihood of observing a particular residue at a given position can be calculated. Without being bound by a particular theory, positions in sequences with high variability may be amenable to mutation (e.g., PSSM score ≥0) to produce functional homologs.

PSSM may be paired with calculation of a Rosetta energy function, which determines the difference between the wild-type and the single-point mutant. The Rosetta energy function calculates this difference as (ΔΔG_{calc}). With the Rosetta function, the bonding interactions between a mutated residue and the surrounding atoms are used to determine whether a mutation increases or decreases protein stability. For example, a mutation that is designated as favorable by the PSSM score (e.g. PSSM score ≥0), can then be analyzed using the Rosetta energy function to determine the potential impact of the mutation on protein stability. Without being bound by a particular theory, potentially stabilizing mutations are desirable for protein engineering (e.g., production of functional homologs). In some embodiments, a potentially stabilizing mutation has a ΔΔG_{calc} value of less than −0.1 (e.g., less than −0.2, less than −0.3, less than −0.35, less than −0.4, less than −0.45, less than −0.5, less than −0.55, less than −0.6, less than −0.65, less than −0.7, less than −0.75, less than −0.8, less than −0.85, less than −0.9, less than −0.95, or less than −1.0) Rosetta energy units (R.e.u.). See, e.g., Goldenzweig et al., Mol Cell. 2016 Jul 21;63(2):337-346. Doi: 10.1016/j.molcel2016.06.012.

In some embodiments, a 1-SST, 1-FFT, or 6-SFT enzyme coding sequence comprises a mutation at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 positions corresponding to a reference (e.g., 1-SST, 1-FFT, or 6-SFT enzyme) coding sequence. In some embodiments, the 1-SST, 1-FFT, or 6-SFT enzyme coding sequence comprises a mutation in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99,100 or more codons of the coding sequence relative to a reference (e.g., 1-SST, 1-FFT, or 6-SFT enzyme) coding sequence. As will be understood by one of ordinary skill in the art, a mutation within a codon may or may not change the amino acid that is encoded by the codon due to degeneracy of the genetic code. In some embodiments, the one or more mutations in the coding sequence do not alter the amino acid sequence of the coding sequence (e.g., 1-SST, 1-FFT, or 6-SFT enzyme) relative to the amino acid sequence of a reference polypeptide (e.g., 1-SST, 1-FFT, or 6-SFT enzyme).

In some embodiments, the one or more mutations in a recombinant 1-SST, 1-FFT, or 6-SFT enzyme sequence alters the amino acid sequence of the polypeptide (e.g., 1-SST, 1-FFT, or 6-SFT enzyme) relative to the amino acid sequence of a reference polypeptide (e.g., 1-SST, 1-FFT, or 6-SFT enzyme). In some embodiments, the one or more mutations alters the amino acid sequence of the recombinant polypeptide (e.g., 1-SST, 1-FFT, or 6-SFT enzyme) relative to the amino acid sequence of a reference polypeptide (e.g., 1-SST, 1-FFT, or 6-SFT enzyme) and alters (enhances or reduces) an activity of the polypeptide relative to the reference polypeptide.

The activity, including specific activity, of any of the recombinant polypeptides described in this application (e.g., 1-SST, 1-FFT, or 6-SFT enzyme) may be measured using routine methods. As a non-limiting example, a recombinant polypeptide's activity may be determined by measuring its substrate specificity, product(s) produced, the concentration of product(s) produced, or any combination thereof. As used in this application, “specific activity” of a recombinant polypeptide refers to the amount (e.g., concentration) of a particular product produced for a given amount (e.g., concentration) of the recombinant polypeptide per unit time.

The skilled artisan will also realize that mutations in a recombinant polypeptide (e.g., 1-SST, 1-FFT, or 6-SFT enzyme) coding sequence may result in conservative amino acid substitutions that provide functionally equivalent variants of the foregoing polypeptides, e.g., variants that retain the activities of the polypeptides. As used in this application, a “conservative amino acid substitution” refers to an amino acid substitution that does not alter the relative charge or size characteristics or functional activity of the protein in which the amino acid substitution is made.

In some instances, an amino acid is characterized by its R group (see, e.g., Table 1). For example, an amino acid may comprise a nonpolar aliphatic R group, a positively charged R group, a negatively charged R group, a nonpolar aromatic R group, or a polar uncharged R group. Non-limiting examples of an amino acid comprising a nonpolar aliphatic R group include alanine, glycine, valine, leucine, methionine, and isoleucine. Non-limiting examples of an amino acid comprising a positively charged R group includes lysine, arginine, and histidine. Non-limiting examples of an amino acid comprising a negatively charged R group include aspartate and glutamate. Non-limiting examples of an amino acid comprising a nonpolar, aromatic R group include phenylalanine, tyrosine, and tryptophan. Non-limiting examples of an amino acid comprising a polar uncharged R group include serine, threonine, cysteine, proline, asparagine, and glutamine.

Non-limiting examples of functionally equivalent variants of polypeptides may include conservative amino acid substitutions in the amino acid sequences of proteins disclosed in this application. As used in this application “conservative substitution” is used interchangeably with “conservative amino acid substitution” and refers to any one of the amino acid substitutions provided in Table 1.

In some embodiments, 1, 2, 3, 4, 5, 6, 7, 8, 9, 10, 11, 12, 13, 14, 15, 16, 17, 18, 19, 20 or more than 20 residues can be changed when preparing variant polypeptides. In some embodiments, amino acids are replaced by conservative amino acid substitutions.

TABLE 1
Conservative Amino Acid Substitutions.

| Original | | Conservative Amino |
|---|---|---|
| Residue | R Group Type | Acid Substitutions |
| Ala | nonpolar aliphatic R group | Cys, Gly, Ser |
| Arg | positively charged R group | His, Lys |
| Asn | polar uncharged R group | Asp, Gln, Glu |
| Asp | negatively charged R group | Asn, Gln, Glu |
| Cys | polar uncharged R group | Ala, Ser |
| Gln | polar uncharged R group | Asn, Asp, Glu |
| Glu | negatively charged R group | Asn, Asp, Gln |
| Gly | nonpolar aliphatic R group | Ala, Ser |
| His | positively charged R group | Arg, Tyr, Trp |
| Ile | nonpolar aliphatic R group | Leu, Met, Val |
| Leu | nonpolar aliphatic R group | Ile, Met, Val |
| Lys | positively charged R group | Arg, His |
| Met | nonpolar aliphatic R group | Ile, Leu, Phe, Val |
| Pro | polar uncharged R group | |
| Phe | nonpolar aromatic R group | Met, Trp, Tyr |
| Ser | polar uncharged R group | Ala, Gly, Thr |
| Thr | polar uncharged R group | Ala, Asn, Ser |
| Trp | nonpolar aromatic R group | His, Phe, Tyr, Met |
| Tyr | nonpolar aromatic R group | His, Phe, Trp |
| Val | nonpolar aliphatic R group | Ile, Leu, Met, Thr |

Amino acid substitutions in the amino acid sequence of a polypeptide to produce a recombinant polypeptide variant having a desired property and/or activity can be made by alteration of the coding sequence of the polypeptide. Similarly, conservative amino acid substitutions in the amino acid sequence of a polypeptide to produce functionally equivalent variants of the polypeptide typically are made by alteration of the coding sequence of the recombinant polypeptide.

A sequence encoding an enzyme of the present disclosure may further encode a secretion signal. As a non-limiting example, a secretion signal may be selected based on the host cell of interest. In some embodiments, a secretion signal may be a yeast, plant, or bacteria secretion signal.

In some embodiments, a secretion signal comprises a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical to:

(SEQ ID NO: 60)
MRFPSIFTAVLFAASSALAAPVNTTTEDETAQIPAEAVIGYSDLEGDFDV
AVLPFSNSTNNGLLFINTTIASIAAKEEGVSLEKREAEA.

In some embodiments, nucleic acid sequence encoding a secretion signal comprises a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical to:

(SEQ ID NO: 61)
ATGAGATTTCCTTCAATTTTTACTGCTGTTTTATTCGCAGCATCCTCCGC
ATTAGCTGCTCCAGTCAACACTACAACAGAAGATGAAACGGCACAAATTC
CGGCTGAAGCTGTCATCGGTTACTCAGATTTAGAAGGGGATTTCGATGTT
GCTGTTTTGCCATTTTCCAACAGCACAAATAACGGGTTATTGTTTATAAA
TACTACTATTGCCAGCATTGCTGCTAAAGAAGAAGGGGTATCTCTCGAGA
AAAGAGAGGCTGAAGCT.

It should be appreciated that other secretion signals known to one of ordinary skill in the art would also be compatible with aspects of the disclosure.

Nucleic Acids Encoding Enzymes of the Disclosure

Aspects of the present disclosure relate to recombinant enzymes, functional modifications and variants thereof, as well as uses relating thereto. For example, the enzymes and cells described in this application may be used to promote production of fructans, e.g., branched fructans, e.g., branched inulins. The methods may comprise using a host cell comprising one or more enzymes disclosed in this application, a cell lysate, isolated enzymes, or any combination thereof. Methods comprising recombinant expression of polynucleotides encoding an enzyme disclosed in this application in a host cell are encompassed by the present disclosure. In vitro methods comprising reacting one or more enzymes for the production of polyfructans in a reaction mixture with a BCAA pathway enzyme disclosed in this application are also encompassed by the disclosure. In some embodiments, the BCAA pathway enzyme is an 1-SST, 1-FFT, or 6-SFT enzyme, or a combination thereof.

A nucleic acid encoding any one or more of the recombinant polypeptides 1-SST, 1-FFT, and/or 6-SFT is encompassed by the disclosure and may be comprised within a host cell. In some embodiments, the nucleic acid is in the form of an operon. In some embodiments, at least one ribosome binding site is present between one or more the coding sequences present in the nucleic acid.

In some embodiments, a nucleic acid provided in this application is a nucleic acid that hybridizes under high or medium stringency conditions to a nucleic acid encoding a 1-SST, 1-FFT, and/or 6-SFT, and that is biologically active. For example, high stringency conditions can include 0.2 to 1×SSC at 65° C. followed by a wash at 0.2×SSC at 65° C. In some embodiments, a nucleic acid provided in this application is a nucleic acid that hybridizes under low stringency conditions to a nucleic acid encoding a 1-SST, 1-FFT, and/or 6-SFT, and that is biologically active. For example, low stringency conditions can include 6×SSC at room temperature followed by a wash at 2×SSC at room temperature. Other hybridization conditions include 3×SSC at 40° C. or 50° C., followed by a wash in 1 or 2×SSC at 20° C., 30° C., 40° C., 50° C., 60° C., or 65° C.

Hybridizations can be conducted in the presence of formaldehyde, e.g., 10%, 20%, 30% 40% or 50%, which further increases the stringency of hybridization. Theory and practice of nucleic acid hybridization is described, e.g., in S. Agrawal (ed.) Methods in Molecular Biology, volume 20; and Tijssen (1993) Laboratory Techniques in biochemistry and molecular biology-hybridization with nucleic acid probes, e.g., part I chapter 2 “Overview of principles of hybridization and the strategy of nucleic acid probe assays,” Elsevier, New York. Exemplary proteins may have at least about 50%, 70%, 80%, 90%, 95%, 98% or 99% homology or identity with a 1-SST, 1-FFT, or 6-SFT protein or a domain thereof, e.g., a catalytic domain. Other exemplary proteins may be encoded by a nucleic acid that has at least about 50%, 70%, 80%, 90%, 95%, 98% or 99% homology or identity with a nucleic acid encoding a 1-SST, 1-FFT, or 6-SFT protein or a domain thereof, e.g., a catalytic domain.

A nucleic acid encoding any one or more of the recombinant polypeptides described in this application may be incorporated into any appropriate vector through any method known in the art. For example, the vector may be an expression vector, including but not limited to a viral vector (e.g., a lentiviral, retroviral, adenoviral, or adeno-associated viral vector), any vector suitable for transient expression, any vector suitable for constitutive expression, or any vector suitable for inducible expression (e.g., a galactose-inducible or doxycycline-inducible vector).

In some embodiments, a vector replicates autonomously in the cell. In some embodiments, a vector integrates into a chromosome within a cell. A vector can contain one or more endonuclease restriction sites that are cut by a restriction endonuclease to insert and ligate a nucleic acid containing a gene described in this application to produce a recombinant vector that is able to replicate in a cell. Vectors are typically composed of DNA, although RNA vectors are also available. Cloning vectors include, but are not limited to: plasmids, fosmids, phagemids, virus genomes and artificial chromosomes. As used in this application, the terms “expression vector” or “expression construct” refer to a nucleic acid construct, generated recombinantly or synthetically, with a series of specified nucleic acid elements that permit transcription of a particular nucleic acid in a host cell (e.g., microbe), such as a yeast cell. In some embodiments, the nucleic acid sequence of a gene described in this application is inserted into a cloning vector such that it is operably joined to regulatory sequences and, in some embodiments, expressed as an RNA transcript. In some embodiments, the vector contains one or more markers, such as a selectable marker, to identify cells transformed or transfected with the recombinant vector. In some embodiments, the nucleic acid sequence of a gene described in this application is recoded. Recoding may increase production of the gene product by at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, including all values in between) relative to a reference sequence that is not recoded.

A coding sequence and a regulatory sequence are said to be “operably joined” or “operably linked” when the coding sequence and the regulatory sequence are covalently linked and the expression or transcription of the coding sequence is under the influence or control of the regulatory sequence. If the coding sequence is to be translated into a functional protein, the coding sequence and the regulatory sequence are said to be operably joined if induction of a promoter in the 5′ regulatory sequence permits the coding sequence to be transcribed and if the nature of the linkage between the coding sequence and the regulatory sequence does not (1) result in the introduction of a frame-shift mutation, (2) interfere with the ability of the promoter region to direct the transcription of the coding sequence, or (3) interfere with the ability of the corresponding RNA transcript to be translated into a protein.

In some embodiments, the nucleic acid encoding any one or more of the proteins described in this application is under the control of regulatory sequences (e.g., enhancer sequences). In some embodiments, a nucleic acid is expressed under the control of a promoter. The promoter can be a native promoter, e.g., the promoter of the gene in its endogenous context, which provides normal regulation of expression of the gene. Alternatively, a promoter can be a promoter that is different from the native promoter of the gene, e.g., the promoter is different from the promoter of the gene in its endogenous context.

Enzymes disclosed herein can be encoded by the same heterologous polynucleotide or by different heterologous polynucleotides. For example, at least 2, 3, 4, 5, 6, 7, 8, 9, 10, or more than 10 enzymes can be encoded by the same heterologous polynucleotide or can be encoded by one or more different heterologous polynucleotides.

In some embodiments, a heterologous polynucleotide encoding a 1-SST enzyme also encodes a 1-FFT and/or a 6-SFT enzyme; a heterologous polynucleotide encoding a 1-FFT enzyme also encodes a 1-SST enzyme and/or a 6-SFT enzyme; or a heterologous polynucleotide encoding a 6-SFT enzyme also encodes a 1-SST enzyme and/or a 1-FFT enzyme.

In some embodiments, a heterologous polynucleotide comprises a single promoter operably linked to a polynucleotide encoding at least one enzyme. For example, a single nucleic acid encoding at least 1, at least 2, at least 3, at least 4, at least 5, at least 6, at least 7, at least 8, at least 9, or at least 10 enzymes may be operably linked to a single promoter. Expression of enzymes within a single heterologous polynucleotide may be controlled by any method known in the art, including, for example, by internal ribosome entry sites (IRES) or polypeptide cleavage signals such as 2A sequences.

In some instances, a heterologous polynucleotide comprises more than one promoter. In some instances, separate promoters are operably linked to at least two polynucleotide sequences that each encode an enzyme used to produce a polyfructan. In some instances, separate promoters are operably linked to each polynucleotide sequence encoding an enzyme used to produce a polyfructan.

In some embodiments, the promoter is a eukaryotic promoter. Non-limiting examples of eukaryotic promoters include TDH3, PGK1, PKC1, PDC1, TEF1, TEF2, RPL18B, SSA1, TDH2, PYK1,TPI1 GAL1, GAL10, GAL7, GAL3, GAL2, MET3, MET25, HXT3, HXT7, ACT1, 6-SFT1, 6-SFT2, CUP1-1, ENO2, pAOX1, pGAP1, and SOD1, as would be known to one of ordinary skill in the art (see, e.g., Addgene website: blog.addgene.org/plasmids-101-the-promoter-region). In some embodiments, the promoter is a prokaryotic promoter (e.g., bacteriophage or bacterial promoter). Non-limiting examples of bacteriophage promoters include Pls icon, T3, T7, SP6, and PL. Non-limiting examples of bacterial promoters include Pbad, PmgrB, Ptrc2, Plac/ara, Ptac, and Pm.

In some embodiments, the promoter is an inducible promoter. As used in this application, an “inducible promoter” is a promoter controlled by the presence or absence of a molecule. This may be used, for example, to controllably induce the expression of an enzyme. In some instances, an inducible promoter is used to controllably repress expression of an enzyme. Non-limiting examples of inducible promoters include chemically regulated promoters and physically regulated promoters. For chemically regulated promoters, the transcriptional activity can be regulated by one or more compounds, such as alcohol, tetracycline, galactose, a steroid, a metal, or other compounds. For physically regulated promoters, transcriptional activity can be regulated by a phenomenon such as light or temperature. Non-limiting examples of tetracycline-regulated promoters include anhydrotetracycline (aTc)-responsive promoters and other tetracycline-responsive promoter systems (e.g., a tetracycline repressor protein (tetR), a tetracycline operator sequence (tetO) and a tetracycline transactivator fusion protein (tTA)). Non-limiting examples of steroid-regulated promoters include promoters based on the rat glucocorticoid receptor, human estrogen receptor, moth ecdysone receptors, and promoters from the steroid/retinoid/thyroid receptor superfamily. Non-limiting examples of metal-regulated promoters include promoters derived from metallothionein (proteins that bind and sequester metal ions) genes. Non-limiting examples of pathogenesis-regulated promoters include promoters induced by salicylic acid, ethylene or benzothiadiazole (BTH). Non-limiting examples of temperature/heat-inducible promoters include heat shock promoters. Non-limiting examples of light-regulated promoters include light responsive promoters from plant cells. In certain embodiments, the inducible promoter is a galactose-inducible promoter. In some embodiments, the inducible promoter is induced by one or more physiological conditions (e.g., pH, temperature, radiation, osmotic pressure, saline gradients, cell surface binding, or concentration of one or more extrinsic or intrinsic inducing agents). Non-limiting examples of an extrinsic inducer or inducing agent include amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or any combination thereof. In some embodiments, an inducible promoter is the pAOX1 promoter. In some embodiments, an inducible promoter is used to drive expression in a eukaryotic cell. In some embodiments, a eukaryotic cell is a yeast cell. In some embodiments, a yeast cell is a Pichia cell. In some embodiments, a yeast cell is a Saccharomyces cell.

In some embodiments, the promoter is a constitutive promoter. As used in this application, a “constitutive promoter” refers to an unregulated promoter that allows continuous transcription of a gene. Non-limiting examples of a constitutive promoter include TDH3, PGK1, PKC1, PDC1, TEF1, TEF2, RPL18B, SSA1, TDH2, PYK1,TPI1, HXT3, HXT7, ACT1, 6-SFT1, 6-SFT2, ENO2, pGAP1, and SOD1. In some embodiments, a constitutive promoter is used to drive expression in a eukaryotic cell. In some embodiments, a eukaryotic cell is a yeast cell. In some embodiments, a yeast cell is a Pichia cell. In some embodiments, a yeast cell is a Saccharomyces cell.

Other inducible promoters or constitutive promoters known to one of ordinary skill in the art are also compatible with aspects of the disclosure.

The precise nature of the regulatory sequences needed for gene expression may vary between species or cell types, but generally can include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences can include a promoter region which includes a promoter sequence for transcriptional control of the operably joined gene. Regulatory sequences may also include enhancer sequences or upstream activator sequences. The vectors disclosed in this application may include 5′ leader or signal sequences. The regulatory sequence may also include a terminator sequence. In some embodiments, a terminator sequence marks the end of a gene in DNA during transcription. The choice and design of one or more appropriate vectors suitable for inducing expression of one or more genes described in this application in a heterologous organism is within the ability and discretion of one of ordinary skill in the art.

Expression vectors containing necessary elements for expression are commercially available and known to one of ordinary skill in the art (see, e.g., Sambrook et al., Molecular Cloning: A Laboratory Manual, Fourth Edition, Cold Spring Harbor Laboratory Press, 2012).

Host Cells

Any of the proteins or enzymes of the disclosure may be expressed in a host cell. The term “host cell” refers to a cell that can be used to express a polynucleotide, such as a polynucleotide that encodes an enzyme used in production of oligosaccharides.

he disclosed methods, compositions, and host cells are exemplified with Pichia pastoris cells, but are also applicable to other host cells. In this application, the term “Pichia pastoris” is used interchangeably with the term “Komagataella phaffii.”

Suitable host cells include, but are not limited to: yeast cells, bacterial cells, algal cells, plant cells, fungal cells, insect cells, and animal cells, including mammalian cells. In one illustrative embodiment, suitable host cells include Pichia pastoris.

Suitable yeast host cells include, but are not limited to: Candida, Escherichia, Hansenula, Saccharomyces, Schizosaccharomyces, Pichia, Kluyveromyces, and Yarrowia. In some embodiments, the yeast cell is Escherichia coli, Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, or Yarrowia lipolytica.

In some embodiments, the yeast strain is an industrial polyploid yeast strain. Other non-limiting examples of fungal cells include cells obtained from Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

In certain embodiments, the host cell is an algal cell such as Chlamydomonas (e.g., C. Reinhardtii) and Phormidium (P. sp. ATCC29409).

In other embodiments, the host cell is a prokaryotic cell. Suitable prokaryotic cells include gram positive, gram negative, and gram-variable bacterial cells. The host cell may be a species of, but not limited to: Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Saccharopolyspora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas.

In some embodiments, the bacterial host strain is an industrial strain. Numerous bacterial industrial strains are known and suitable for the methods and compositions described in this application.

In some embodiments, the bacterial host cell is of the Agrobacterium species (e.g., A. radiobacter, A. rhizogenes, A. rubi), the Arthrobacterspecies (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), or the Bacillus species (e.g., B. thuringiensis, B. anthracis, B. megaterium, B. subtilis, B. lentus, B. circulars, B. pumilus, B. lautus, B. coagulans, B. brevis, B. firmus, B. alkaophius, B. licheniformis, B. clausii, B. stearothermophilus, B. halodurans and B. amyloliquefaciens. In particular embodiments, the host cell is an industrial Bacillus strain including but not limited to B. subtilis, B. pumilus, B. licheniformis, B. megaterium, B. clausii, B. stearothermophilus and B. amyloliquefaciens. In some embodiments, the host cell is an industrial Clostridium species (e.g., C. acetobutylicum, C. tetani E88, C. lituseburense, C. saccharobutylicum, C. perfringens, C. beijerinckii). In some embodiments, the host cell is an industrial Corynebacterium species (e.g., C. glutamicum, C. acetoacidophilum). In some embodiments, the host cell is an industrial Escherichia species (e.g., E. coli). In some embodiments, the host cell is an industrial Erwinia species (e.g., E. uredovora, E. carotovora, E. ananas, E. herbicola, E. punctata, E. terreus). In some embodiments, the host cell is an industrial Pantoea species (e.g., P. citrea, P. agglomerans). In some embodiments, the host cell is an industrial Pseudomonas species, (e.g., P. putida, P. aeruginosa, P. mevalonii). In some embodiments, the host cell is an industrial Streptococcus species (e.g., S. equisimiles, S. pyogenes, S. uberis). In some embodiments, the host cell is an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the host cell is an industrial Zymomonas species (e.g., Z. mobilis, Z. lipolytica).

The present disclosure is also suitable for use with a variety of animal cell types, including mammalian cells, for example, human (including 293, HeLa, WI38, PER.C6 and Bowes melanoma cells), mouse (including 3T3, NSO, NS1, Sp2/0), hamster (CHO, BHK), monkey (COS, FRhL, Vero), and hybridoma cell lines.

In various embodiments, strains that may be used in the practice of the disclosure including both prokaryotic and eukaryotic strains, are readily accessible to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL). The present disclosure is also suitable for use with a variety of plant cell types.

The term “cell,” as used in this application, may refer to a single cell or a population of cells, such as a population of cells belonging to the same cell line or strain. Use of the singular term “cell” should not be construed to refer explicitly to a single cell rather than a population of cells. The host cell may comprise genetic modifications relative to a wild-type counterpart.

A vector encoding any one or more of the recombinant polypeptides (e.g., 1-SST, 1-FFT, and/or 6-SFT) described in this application may be introduced into a suitable host cell using any method known in the art. Host cells may be cultured under any conditions suitable as would be understood by one of ordinary skill in the art. For example, any media, temperature, and incubation conditions known in the art may be used. For host cells carrying an inducible vector, cells may be cultured with an appropriate inducible agent to promote expression.

Any of the cells disclosed in this application can be cultured in media of any type (rich or minimal) and any composition prior to, during, and/or after contact and/or integration of a nucleic acid. The conditions of the culture or culturing process can be optimized through routine experimentation as would be understood by one of ordinary skill in the art. In some embodiments, the selected media is supplemented with various components. In some embodiments, the concentration and amount of a supplemental component is optimized. In some embodiments, other aspects of the media and growth conditions (e.g., pH, temperature, etc.) are optimized through routine experimentation. In some embodiments, the frequency that the media is supplemented with one or more supplemental components, and the amount of time that the cell is cultured, is optimized.

Culturing of the cells described in this application can be performed in culture vessels known and used in the art. In some embodiments, an aerated reaction vessel (e.g., a stirred tank reactor) is used to culture the cells. In some embodiments, a bioreactor or fermentor is used to culture the cell. Thus, in some embodiments, the cells are used in fermentation. The terms “bioreactor” and “fermentor” are interchangeably used in this application and refer to an enclosure, or partial enclosure, in which a biological, biochemical and/or chemical reaction takes place, involving a living organism or part of a living organism, including one or more secreted enzymes. A “large-scale bioreactor” or “industrial-scale bioreactor” is a bioreactor that is used to generate a product on a commercial or quasi-commercial scale. Large scale bioreactors typically have volumes in the range of liters, hundreds of liters, thousands of liters, or more.

In some embodiments, methods of culturing cell(s) of the present disclosure comprise overexpression of an enzyme described in this application. In some embodiments, methods of culturing cell(s) further comprise isolating or purifying enzymes expressed from the cell(s) (e.g., isolating enzymes following secretion of the enzymes by the cells).

Non-limiting examples of bioreactors include: stirred tank fermentors, bioreactors agitated by rotating mixing devices, chemostats, bioreactors agitated by shaking devices, airlift fermentors, packed-bed reactors, fixed-bed reactors, fluidized bed bioreactors, bioreactors employing wave induced agitation, centrifugal bioreactors, roller bottles, and hollow fiber bioreactors, roller apparatuses (for example benchtop, cart-mounted, and/or automated varieties), vertically-stacked plates, spinner flasks, stirring or rocking flasks, shaken multi-well plates, MD bottles, T-flasks, Roux bottles, multiple-surface tissue culture propagators, modified fermentors, and coated beads (e.g., beads coated with serum proteins, nitrocellulose, or carboxymethyl cellulose to prevent cell attachment).

In some embodiments, the bioreactor includes a cell culture system where the cell (e.g., bacterial cell) is in contact with moving liquids and/or gas bubbles. In some embodiments, the cell or cell culture is grown in suspension. In other embodiments, the cell or cell culture is attached to a solid phase carrier. Non-limiting examples of a carrier system includes microcarriers (e.g., polymer spheres, microbeads, and microdisks that can be porous or non-porous), cross-linked beads (e.g., dextran) charged with specific chemical groups (e.g., tertiary amine groups), 2D microcarriers including cells trapped in nonporous polymer fibers, 3D carriers (e.g., carrier fibers, hollow fibers, multicartridge reactors, and semi-permeable membranes that can comprising porous fibers), microcarriers having reduced ion exchange capacity, encapsulation cells, capillaries, and aggregates. In some embodiments, carriers are fabricated from materials such as dextran, gelatin, glass, or cellulose.

In some embodiments, industrial-scale processes are operated in continuous, semi-continuous or non-continuous modes. Non-limiting examples of operation modes are batch, fed batch, extended batch, repetitive batch, draw/fill, rotating-wall, spinning flask, and/or perfusion mode of operation. In some embodiments, a bioreactor allows continuous or semi-continuous replenishment of the substrate stock, for example a carbohydrate source and/or continuous or semi-continuous separation of the product, from the bioreactor.

In some embodiments, the bioreactor or fermentor includes a sensor and/or a control system to measure and/or adjust reaction parameters. Non-limiting examples of reaction parameters include biological parameters (e.g., growth rate, cell size, cell number, cell density, cell type, or cell state, etc.), chemical parameters (e.g., pH, redox-potential, concentration of reaction substrate and/or product, concentration of dissolved gases, such as oxygen concentration and CO₂ concentration, nutrient concentrations, metabolite concentrations, concentration of an oligopeptide, concentration of an amino acid, concentration of a vitamin, concentration of a hormone, concentration of an additive, serum concentration, ionic strength, concentration of an ion, relative humidity, molarity, osmolarity, concentration of other chemicals, for example buffering agents, adjuvants, or reaction by-products), physical/mechanical parameters (e.g., density, conductivity, degree of agitation, pressure, and flow rate, shear stress, shear rate, viscosity, color, turbidity, light absorption, mixing rate, conversion rate, as well as thermodynamic parameters, such as temperature, light intensity/quality, etc.). Sensors to measure the parameters described in this application are well known to one of ordinary skill in the relevant mechanical and electronic arts. Control systems to adjust the parameters in a bioreactor based on the inputs from a sensor described in this application are well known to one of ordinary skill in the art of bioreactor engineering.

In some embodiments, methods involve batch fermentation (e.g., shake flask fermentation). General considerations for batch fermentation (e.g., shake flask fermentation) include the level of oxygen and glucose. For example, batch fermentation (e.g., shake flask fermentation) may be oxygen and glucose limited, so in some embodiments, the capability of a strain to perform in a well-designed fed-batch fermentation is underestimated. Also, the final product may display some differences from the substrate in terms of solubility, toxicity, cellular accumulation and secretion and in some embodiments can have different fermentation kinetics.

In some embodiments, the cells of the present disclosure are adapted to consume sucrose and produce fructans in vivo. In some embodiments, the cells are adapted to produce one or more enzymes for sucrose consumption via conversion to 1-kestose, 6-kestose, and/or inulin (e.g., 1-SST, 1-FFT, and/or 6-SFT). In such embodiments, the enzyme can catalyze reactions for the consumption of sucrose by bioconversion in an in vitro process.

In some embodiments, the cell(s) (e.g., host cell(s)) of the present disclosure comprise one or more heterologous polynucleotides encoding a 1-SST enzyme; a 1-FFT enzyme; and/or a 6-SFT enzyme. In some embodiments, a host cell comprises one or more heterologous polynucleotides encoding a 1-SST enzyme and a 1-FFT enzyme. In some embodiments, a host cell comprises one or more heterologous polynucleotides encoding a 1-SST enzyme and a 6-SFT enzyme. In some embodiments, a host cell comprises one or more heterologous polynucleotides encoding a 1-FFT enzyme and a 6-SFT enzyme. In some embodiments, a host cell comprises one or more heterologous polynucleotides encoding a 1-SST enzyme, a 1-FFT enzyme, and a 6-SFT enzyme.

The term “heterologous” with respect to a polynucleotide, such as a polynucleotide comprising a gene, is used interchangeably with the term “exogenous” and the term “recombinant” and refers to: a polynucleotide that has been artificially supplied to a biological system; a polynucleotide that has been modified within a biological system, or a polynucleotide whose expression or regulation has been manipulated within a biological system. A heterologous polynucleotide that is introduced into or expressed in a host cell may be a polynucleotide that comes from a different organism or species from the host cell, or may be a synthetic polynucleotide, or may be a polynucleotide that is also endogenously expressed in the same organism or species as the host cell. For example, a polynucleotide that is endogenously expressed in a host cell may be considered heterologous when it is situated non-naturally in the host cell; expressed recombinantly in the host cell, either stably or transiently; modified within the host cell; selectively edited within the host cell; expressed in a copy number that differs from the naturally occurring copy number within the host cell; or expressed in a non-natural way within the host cell, such as by manipulating regulatory regions that control expression of the polynucleotide. In some embodiments, a heterologous polynucleotide is a polynucleotide that is endogenously expressed in a host cell but whose expression is driven by a promoter that does not naturally regulate expression of the polynucleotide. In other embodiments, a heterologous polynucleotide is a polynucleotide that is endogenously expressed in a host cell and whose expression is driven by a promoter that does naturally regulate expression of the polynucleotide, but the promoter or another regulatory region is modified. In some embodiments, the promoter is recombinantly activated or repressed. For example, gene-editing based techniques may be used to regulate expression of a polynucleotide, including an endogenous polynucleotide, from a promoter, including an endogenous promoter. See, e.g., Chavez et al., Nat Methods. 2016 Jul; 13(7): 563-567. A heterologous polynucleotide may comprise a wild-type sequence or a mutant sequence as compared with a reference polynucleotide sequence.

Methods

In some aspects, the disclosure provides methods comprising culturing a host cell described in this application (e.g., a host cell comprising a heterologous polynucleotide encoding at least one enzyme selected from the group consisting of 1-SST, 1-FFT, and 6-SFT). In some embodiments, the disclosure provides a method of producing fructans, e.g., inulins, from sucrose comprising culturing a host cell described in this application (e.g., a host cell comprising a heterologous polynucleotide encoding 1-SST, 1-FFT, and/or 6-SFT). In some embodiments, the production and culturing occurs in vivo. In some embodiments, production of one or more products occurs in vitro. In some embodiments, methods of producing fructans using host cells comprise secretion of expressed enzymes (e.g., 1-SST, 1-FFT, and/or 6-SFT) from the cells. Methods involving secreted enzymes may comprise contacting the secreted enzymes with sucrose in the media or in solution surrounding the host cells.

In some aspects, the disclosure provides methods of using isolated or purified enzymes. Non-limiting methods for protein purification may be found, e.g., in Janson, Protein purification: principles, high resolution methods, and applications, Third Edition (2011). In some embodiments, the disclosure provides a method comprising contacting (or incubating) saccharides with one or more enzymes described in this application to produce fructans. In some embodiments, methods of producing fructans comprise contacting saccharides (e.g., sucrose) with one or more of: a 1-SST enzyme; a 1-FFT enzyme; and a 6-SFT enzyme. In some embodiments, methods of producing fructans comprise contacting or incubating saccharides (e.g., sucrose) with a 1-SST enzyme and a 1-FFT enzyme. In some embodiments, methods of producing fructans comprise contacting or incubating saccharides (e.g., sucrose) with a 1-SST enzyme and a 6-SFT enzyme. In some embodiments, methods of producing fructans comprise contacting or incubating saccharides (e.g., sucrose) with a 1-FFT enzyme and a 6-SFT enzyme. In some embodiments, methods of producing fructans comprise contacting or incubating saccharides (e.g., sucrose) with a 1-SST enzyme, a 1-FFT enzyme, and a 6-SFT enzyme.

Production of a fructan may be carried out in a method whereby all the reactions take place in one reactor, such as a bioreactor, which can be referred to as a “one-pot bioconversion.” In some embodiments, at least two enzymes are used in a single reactor. In some embodiments, at least three enzymes are used in a single reactor.

As a non-limiting example of a one-pot bioconversion, in some embodiments, a single strain can be used to secrete multiple enzymes into media containing sucrose to produce a polyfructan. In other embodiments, multiple strains, each encoding one or more enzymes, can be combined into a single fermentation wherein they will each secrete enzymes into media. The secreted enzymes can convert sucrose into branched inulins. Without being bound by a particular theory, glucose and sucrose released from this process can be used to develop increased biomass of the strains and provide additional substrate for the formation of branched inulin. In some instances, a one-pot bioconversion comprises incubation of one or more purified enzymes with a substrate in a single reactor to produce a polyfructan.

In some instances, multiple reactors are used to produce polyfructans. Use of more than one reactor may be referred to as multiple pot bioconversion. In some instances, at least 2, at least 3, at least 4, at least 5, at least 6, at least 7, at least 8, at least 9, or at least 10 reactors are used. As a non-limiting example, a multiple pot bioconversion can comprise incubating isolated 1-SST with sucrose to form kestose. The kestose produced can then be isolated and incubated with 1-FFT and 6-SFT to convert the kestose into branched inulins. The resulting sucrose and glucose can also be isolated and used for host-cell biomass accumulation, for bioconversion, or for alternative processes. In some embodiments, multiple pot bioconversion comprises purification of a product of interest from one reactor and subsequent introduction of the purified product of interest as a substrate in a second reactor.

In some instances, one or more enzymes selected from 1-SST, 1-FFT, and 6-SFT do not comprise a secretion signal. In some instances, the one or more enzymes (e.g., two or more or three or more enzymes) catalyze production of a fructan within a cell by fermentation. For example, a fructan may be produced within a cell and subsequently secreted from the cell, isolated from the cell, or purified from the cell. In some instances, the secreted fructan is the substrate for another reaction. In some instances, the secreted fructan is imported by a cell as a substrate for another reaction. In some instances, a fructan is produced within a cell and subsequently isolated or purified from a cell. The isolated or purified fructan may be used as the substrate for another reaction.

In some aspects, the disclosure provides methods of producing a fructan, comprising first contacting sucrose with a 1-SST enzyme to produce kestose (e.g., 1-kestose); and subsequently contacting kestose (e.g., 1-kestose) with a 1-FFT enzyme and/or a 6-SFT enzyme to produce the fructan. In some embodiments, such a two-step method comprises the use of host cells (e.g., comprising 1-SST, 1-FFT, and/or 6-SFT) and/or the use of isolated enzymes (e.g., 1-SST, 1-FFT, and/or 6-SFT). In some embodiments, kestose produced by contacting sucrose with a 1-SST enzyme is purified prior to being contacted with a 1-FFT enzyme and/or 6-SFT enzyme.

Methods of producing fructans may comprise isolating or purifying said fructans away from host cells and/or enzymes, in accordance with any isolation or purification technique known in the art.

The present invention is further illustrated by the following Examples, which should not be construed as limiting. The entire contents of all of the references (including literature references, issued patents, published patent applications, and co-pending patent applications) cited throughout this application are hereby expressly incorporated by reference. If a reference incorporated in this application contains a term whose definition is incongruous or incompatible with the definition of same term as defined in the present disclosure, the meaning ascribed to the term in this disclosure shall govern. Mention of any reference, article, publication, patent, patent publication, and patent application cited in this application is not, and should not be taken as, an acknowledgment or suggestion that they constitute valid prior art or form part of the common general knowledge of a skilled artisan.

### EXAMPLES

In order that the invention described in this application may be more fully understood, the following examples are set forth. The examples described in this application are offered to illustrate the systems and methods provided in this application and are not to be construed as limiting their scope.

Example 1: Enzyme Library Design and Screening

Enzyme discovery

Machine-learning—based bioinformatics tools were used to identify enzyme candidates for each of the three desired enzymatic activities (1-SST, 1-FFT, and 6-SFT) in public sequence databases (SwissProt and TrEMBL, together known as UniProt). A single library of 152 enzymes was tested for each of the activities.

Library synthesis

DNA sequences for all 1-SST, 1-FFT, and 6-SFT enzymes were coded for expression in Pichia pastoris. Coding sequences were synthesized in an inducible Pichia pastoris expression vector under the control of the T7 promoter.

Cell growth and enzyme preparation

Strains harboring library plasmids were transformed into Pichia pastoris expression host cells. Enzymes were secreted into media, removed from the cells, and concentrated.

Enzyme screening

Bioconversion reactions involved incubating individual enzymes with either sucrose or 1-kestose for 96 hours. The reactions were subsequently stopped by boiling. Samples were subjected to high-performance liquid chromatography and analyzed by a refractive index detector (HPLC-RID).

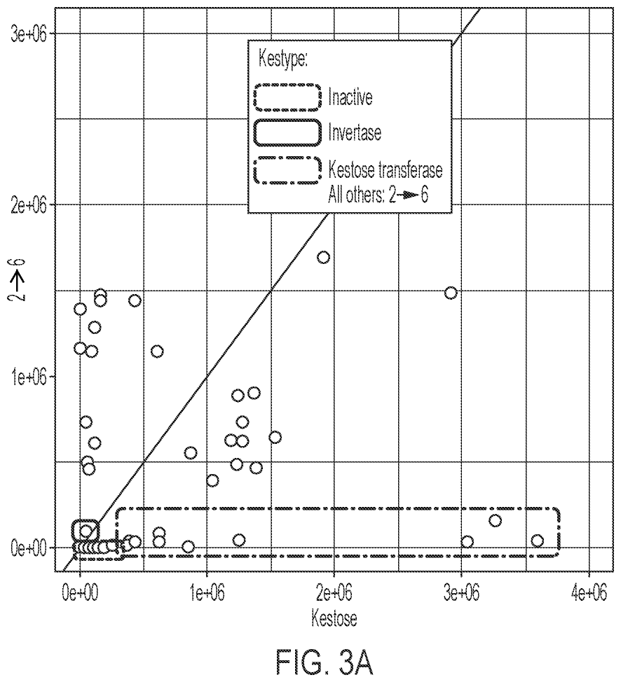

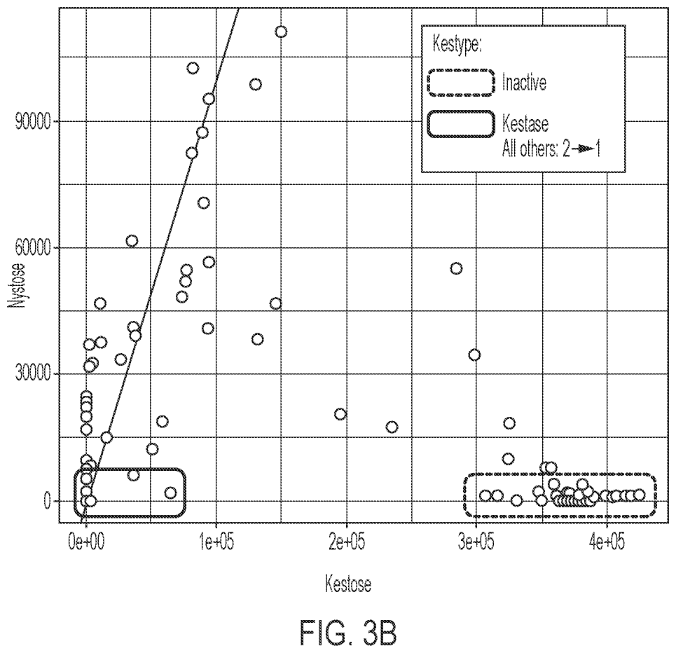

As shown in FIG. 3A, reactions involving incubation of individual enzymes with sucrose provided resultant product mixtures that could be quantified for their concentrations of fructans comprising β(2,6) linkages and fructans comprising β(2,1) linkages (corresponding to 1-kestose). Incubation with sucrose identified enzymes with either 6-SFT or 1-SST activities. 1-SST enzymes produced high levels of 3-sugar oligosaccharides that co-migrated with kestose on HPLC. Incubations with 1-SST did not produce longer sugar polymers. 6-SFT enzymes produced high levels of higher molecular-weight oligosaccharides comprising β(2,6) linkages. Some enzymes that showed minimal activity in polymerizing sucrose demonstrated invertase activity and produced high levels of glucose and fructose.

As shown in FIG. 3B, reactions involving incubation of individual enzymes with 1-kestose provided resultant product mixtures that could be quantified for their concentrations of inulins comprising β(2,1) linkages (labeled ‘Nystose’) and higher-order kestose molecules. Incubation with kestose identified enzymes with 1-FFT activity. Reactions were assayed for high levels of 4+sugar-containing oligosaccharides, resulting in production of sucrose as a by-product. Many enzymes generated these high molecular-weight species. Another class of enzymes-kestases-formed sucrose, but did not show any activity in polymerizing high molecular-weight oligosaccharides.

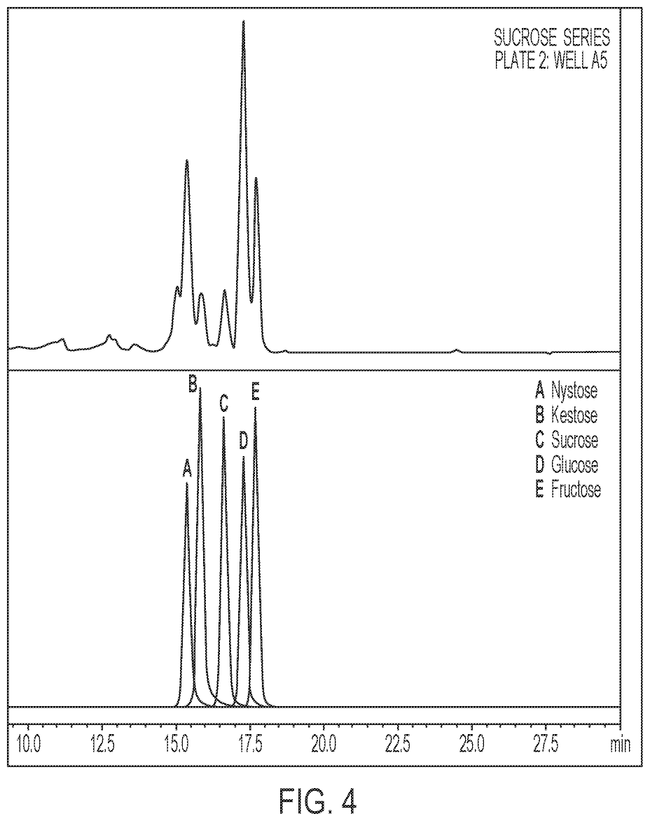

Polyfructans produced were quantified by calculating the area under the curve of the HPLC chromatogram. An example of an HPLC chromatogram of a bioconversion reaction (an individual enzyme incubated with sucrose) is shown in FIG. 4 (top panel). An HPLC chromatogram of a preparation of commercially-available standards is also shown in FIG. 4 (bottom panel).

Example 2: Characterization of high-performing enzymes

Top-performing enzymes were selected for further development. Individual enzymes that showed 6-SFT, 1-SST, or 1-FFT activity in Example 1 were re-expressed, isolated, and assayed for ability to produce fructans. Enzyme preparations were incubated with either sucrose or 1-kestose before bioconversion reactions were analyzed by HPLC-RID and compared to saccharide standards. Peaks were identified by HPLC retention time, and the conversion of sucrose to other sugars was quantified by the relative peak areas from HPLC integrations. Enzymes provided in Table 2 represent the most active of each of the three classes of enzymes (6-SFT, 1-SST, and 1-FFT). “High activity” refers to the highest activity of the proteins that were tested. All proteins were tested for functionality and rank-ordered according to their activity in polymerizing sugars. SEQ ID NOs: 3-4 were modified to include a secretion signal for Pichia pastoris, and the modified constructs (SEQ ID NOs: 25 and 27, respectively) were also identified as having 1-SST activity. SEQ ID NOs: 9-10 were also modified to include a secretion signal for Pichia pastoris, and the modified constructs (SEQ ID NOs: 32 and 34, respectively) were identified as having 1-FFT activity. SEQ ID NOs: 15-21 were also modified to include a secretion signal for Pichia pastoris, and the modified constructs (SEQ ID NOs: 39, 41, 43, 45, 47, 49, and 51, respectively) were identified as having 6-SFT activity.

TABLE 2
Top-Performing Enzymes

| | SEQ ID NO | SEQ ID NO |
|---|---|---|
| Enzyme | (Amino Acid) | (DNA) |
| 1-SST | 1 | 5 |
| 1-FFT | 7 | 11 |
| 6-SFT | 13 | 22 |

Example 3: Bioconversion of sucrose to branched inulin — “One Pot” Bioconversion

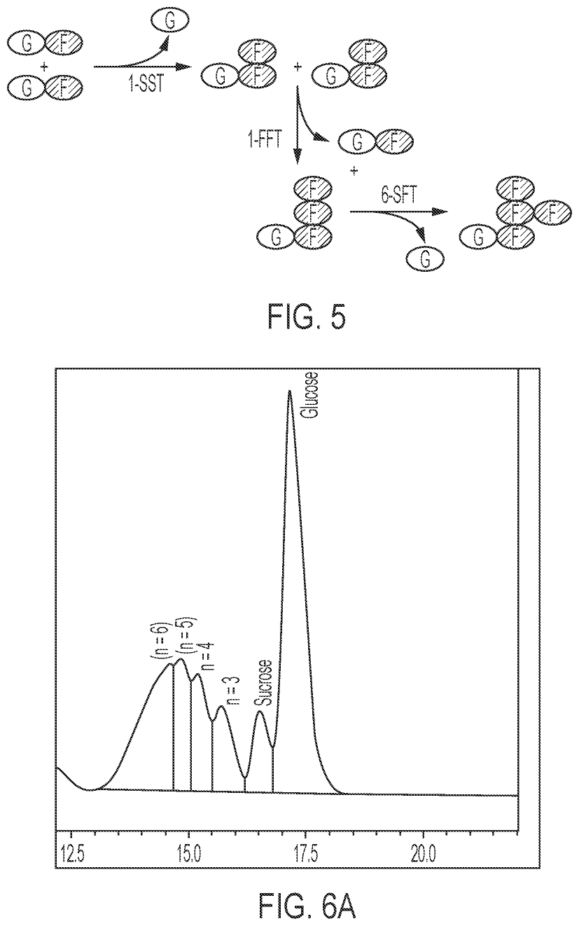

Using the enzymes described in Table 2, a bioconversion of sucrose to branched inulin was performed. As shown in FIG. 5, sucrose (dimer of glucose and fructose) can be converted to 1-kestose (comprising β(2,1) linkage) using a 1-SST enzyme. A 1-FFT enzyme then catalyzes formation of a linear inulin, which itself can be reacted with a 6-SFT enzyme to provide β(2,6) branched inulins.

The three enzymes (1-SST, 1-FFT, and 6-SFT) were combined in a single reaction and incubated with sucrose for 96 hours. After 96 hours, the reaction was stopped by boiling.

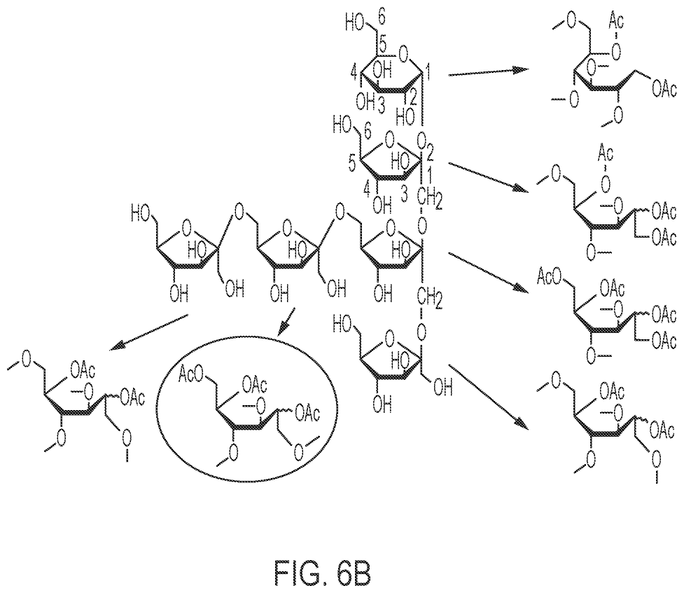

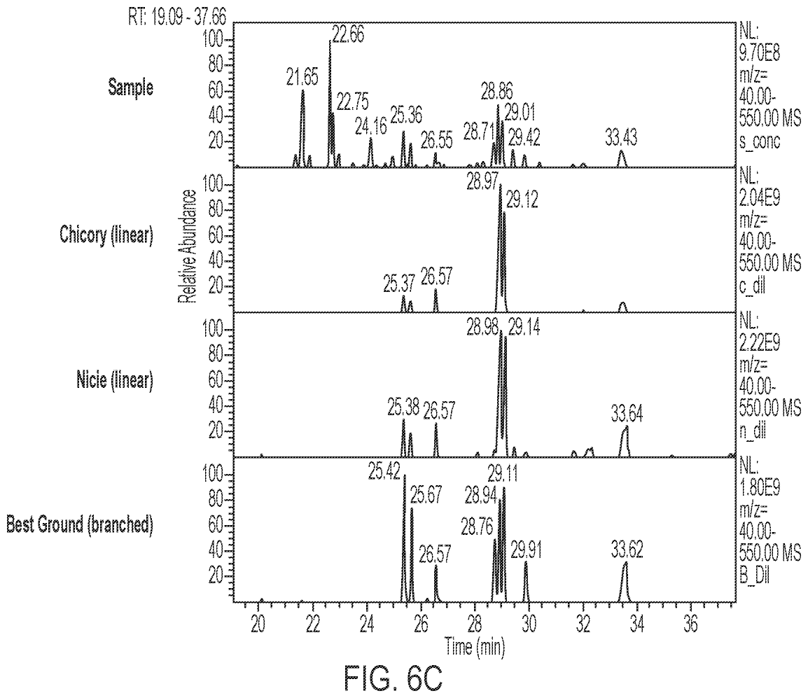

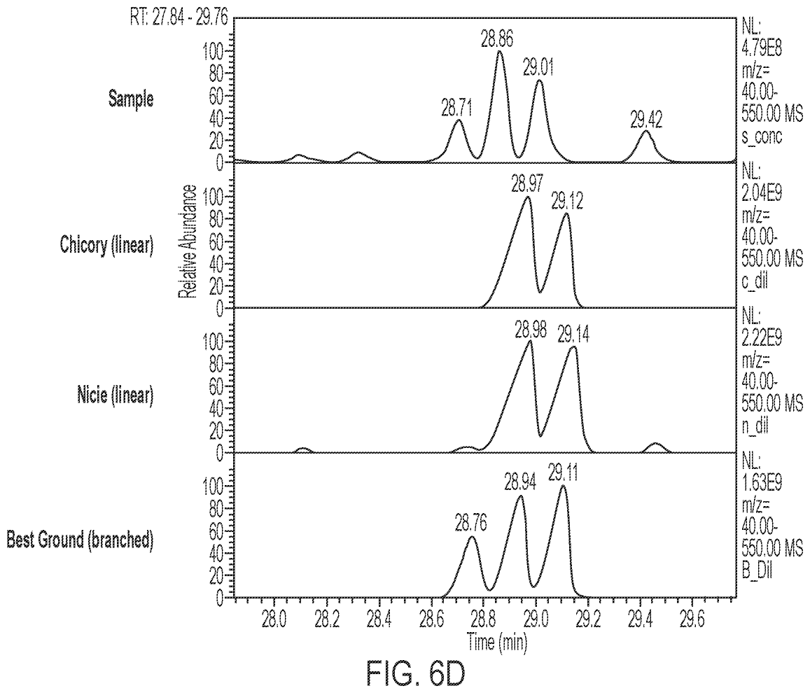

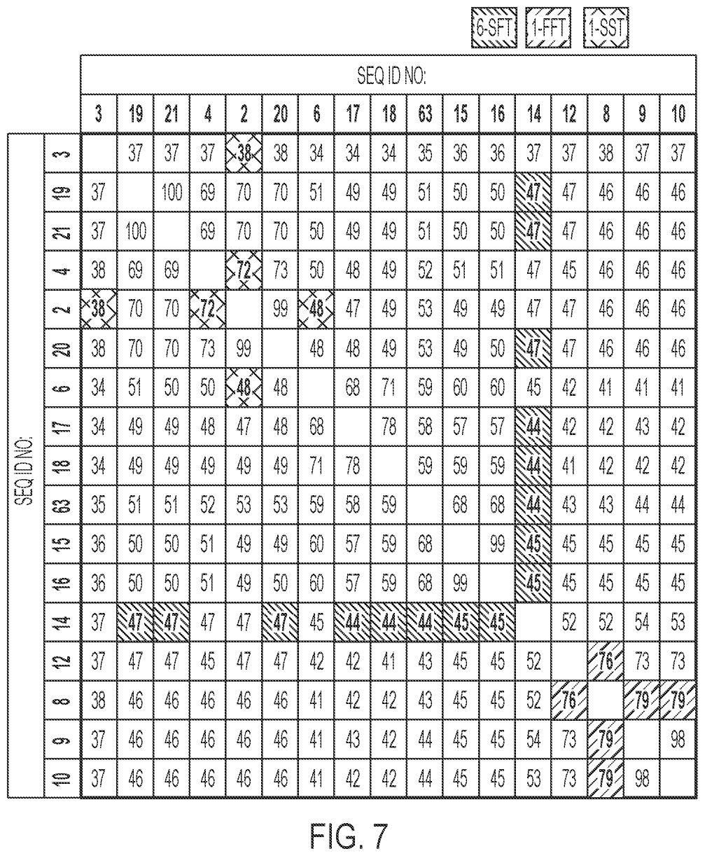

Bioconversion to branched inulin was assayed by HPLC-RID and gas chromatography/mass spectroscopy (GC/MS). Saccharides were identified based on HPLC elution time. As shown in FIG. 6A, higher molecular-weight saccharides (n=3 to n=6) were identified as HPLC peaks that eluted before sucrose. This one-pot conversion reaction showed an increase in glucose formation as well as the formation of early-eluting high-molecular weight material, consistent with the hypothesis that this peak represents branched inulin. Comparison of this material with standards indicated that this was comprised of material with a degree of polymerization greater than 3 (DP3). Glucose did not co-elute with inulin (branched or otherwise). An HPLC assay of reactions showed a high release of glucose as a later-eluting peak in samples where branched inulin was being produced (as an early-eluting peak) (see, e.g., FIG. 6A).

GC/MS was then used to identify the presence of both β(2,1) and β(2,6) linkages in this bioconversion product mixture. Derivatization before GC/MS analysis was performed using a 4-step method that consisted of: 1) methylating free alcoholic -OH groups; 2) hydrolyzing the saccharide linkages; 3) reducing ketone and aldehyde groups; and 4) acylating the alcoholic -OH groups formed during step 3. Following this protocol, the samples were analyzed by GC/MS, which showed a series of products with a well-established elution order and characteristic fragmentation patterns (FIG. 6C-6D). GC/MS of the bioconversion sample resulted in a signature indicative of β(2,6) branched inulin. The bioconversion sample comprised a peak at 28.71 minutes, a peak that is characteristic of a known branched sugar (‘Best Ground’). Notably, this characteristic peak is not found in GC/MS analysis of linear saccharides (Chicory; Nicie).

Example 4: Bioconversion of sucrose to branched inulin—“Two Pot” Bioconversion

An isolated 1-SST enzyme is incubated with sucrose to form kestose. The kestose is isolated and then incubated with 1-FFT and 6-SFT enzymes, which convert the kestose into branched inulins.

The resulting sucrose and glucose can be isolated and used for host-cell biomass accumulation, material of bioconversion, or alternative processes.

Sequences

Non-limiting examples of 1-SST sequences
MRFPSIFTAVLFAASSALAAPVNTTTEDETAQIPAEAVIGYSDLEGDFDVAVLPFSNSTNNGLLFINTTIASIAA
KEEGVSLEKREAEANLMRLRENDYPWTNDMLRWQRTGFHFQPGKNFQADPNAAMFYKGWYHFFYQYNPTGVAWDY
TISWGHAVSKDLLHWNYLPMALRPDHWYDRKGVWSGYSTLLPDGRIVVLYTGGTKELVQVQNLAVPVNLSDPLLL
EWKKSHVNPILVPPPGIEDHDFRDPFPVWYNESDSRWHVVIGSKDPEHYGIVLIYTTKDFVNFTLLPNILHSTKQ
PVGMLECVDLFPVATTDSRANQALDMTTMRPGPGLKYVLKASMDDERHDYYALGSFDLDSFTFTPDDETIDVGIG
LRYDWGKFYASKTFYDQEKQRRVLWGYVGEVDSKRDDALKGWASLQNIPRTILFDTKTKSNLILWPVEEVESLRT
INKNFNSIPLYPGSTYQLDVGEATQLDIVAEFEVDEKAIEATAEADVTYNCSTSGGAANRGVLGPFGLLVLANQE
LSEQTATYFYVSRGIDGNLRTHFCQDELRSSKAGAITKRVVGSTVPVLHGETWALRILVDHSIVESFAQRGRAVA
TSRVYPTEAIYSSARVFLFNNATDAIVTAKTVNVWHINSTYNHVFPGLVAP(SEQ ID NO: 1; secretion
signal is underlined)
MASSTKDVEAPPTLDAPLLGPAAPRSRLRVAPVSLSVMAFLLVAIAAAVLYYNPGGVASNLMRLRENDYPWTNDM
LRWQRTGFHFQPGKNFQADPNAAMFYKGWYHFFYQYNPTGVAWDYTISWGHAVSKDLLHWNYLPMALRPDHWYDR
KGVWSGYSTLLPDGRIVVLYTGGTKELVQVQNLAVPVNLSDPLLLEWKKSHVNPILVPPPGIEDHDFRDPFPVWY
NESDSRWHVVIGSKDPEHYGIVLIYTTKDFVNFTLLPNILHSTKQPVGMLECVDLFPVATTDSRANQALDMTTMR
PGPGLKYVLKASMDDERHDYYALGSFDLDSFTFTPDDETIDVGIGLRYDWGKFYASKTFYDQEKQRRVLWGYVGE
VDSKRDDALKGWASLQNIPRTILFDTKTKSNLILWPVEEVESLRTINKNFNSIPLYPGSTYQLDVGEATQLDIVA
EFEVDEKAIEATAEADVTYNCSTSGGAANRGVLGPFGLLVLANQELSEQTATYFYVSRGIDGNLRTHFCQDELRS
SKAGAITKRVVGSTVPVLHGETWALRILVDHSIVESFAQRGRAVATSRVYPTEAIYSSARVFLFNNATDAIVTAK
TVNVWHINSTYNHVFPGLVAP (SEQ ID NO: 2; secretion signal is underlined)
NLMRLRENDYPWTNDMLRWQRTGFHFQPGKNFQADPNAAMFYKGWYHFFYQYNPTGVAWDYTISWGHAVSKDLLH
WNYLPMALRPDHWYDRKGVWSGYSTLLPDGRIVVLYTGGTKELVQVQNLAVPVNLSDPLLLEWKKSHVNPILVPP
PGIEDHDFRDPFPVWYNESDSRWHVVIGSKDPEHYGIVLIYTTKDFVNFTLLPNILHSTKQPVGMLECVDLFPVA
TTDSRANQALDMTTMRPGPGLKYVLKASMDDERHDYYALGSFDLDSFTFTPDDETIDVGIGLRYDWGKFYASKTF
YDQEKQRRVLWGYVGEVDSKRDDALKGWASLQNIPRTILFDTKTKSNLILWPVEEVESLRTINKNFNSIPLYPGS
TYQLDVGEATQLDIVAEFEVDEKAIEATAEADVTYNCSTSGGAANRGVLGPFGLLVLANQELSEQTATYFYVSRG
IDGNLRTHFCQDELRSSKAGAITKRVVGSTVPVLHGETWALRILVDHSIVESFAQRGRAVATSRVYPTEAIYSSA
RVFLFNNATDAIVTAKTVNVWHINSTYNHVFPGLVAP (SEQ ID NO: 24)
MAKLNRSNIGLSLLLSMFLANFITDLEASSHQDLNQPYRTGYHFQPLKNWMNGPMIYKGIYHLFYQYNPYGAVWD
VRIVWGHSTSVDLVNWISQPPAFNPSQPSDINGCWSGSVTILPNGKPVILYTGIDQNKGQVQNVAVPVNISDPYL
REWSKPPQNPLMTTNAVNGINPDRFRDPTTAWLGRDGEWRVIVGSSTDDRRGLAILYKSRDFFNWTQSMKPLHYE
DLTGMWECPDFFPVSITGSDGVETSSVGENGIKHVLKVSLIETLHDYYTIGSYDREKDVYVPDLGFVQNESAPRL
DYGKYYASKTFYDDVKKRRILWGWVNESSPAKDDIEKGWSGLQSFPRKIWLDESGKELLQWPIEEIETLRGQQVN
WQKKVLKAGSTLQVHGVTAAQADVEVSFKVKELEKADVIEPSWTDPQKICSQGDLSVMSGLGPFGLMVLASNDME
EYTSVYFRIFKSNDDTNKKTKYVVLMCSDQSRSSLNDENDKSTFGAFVAIDPSHQTISLRTLIDHSIVESYGGGG
RTCITSRVYPKLAIGENANLFVFNKGTQSVDILTLSAWSLKSAQINGDLMSPFIEREESRSPNHQF (SEQ ID
NO: 3; secretion signal is underlined)
MRFPSIFTAVLFAASSALAAPVNTTTEDETAQIPAEAVIGYSDLEGDFDVAVLPFSNSTNNGLLFINTTIASIAA
KEEGVSLEKREAEADLNQPYRTGYHFQPLKNWMNGPMIYKGIYHLFYQYNPYGAVWDVRIVWGHSTSVDLVNWIS
QPPAFNPSQPSDINGCWSGSVTILPNGKPVILYTGIDQNKGQVQNVAVPVNISDPYLREWSKPPQNPLMTTNAVN
GINPDRFRDPTTAWLGRDGEWRVIVGSSTDDRRGLAILYKSRDFFNWTQSMKPLHYEDLTGMWECPDFFPVSITG
SDGVETSSVGENGIKHVLKVSLIETLHDYYTIGSYDREKDVYVPDLGFVQNESAPRLDYGKYYASKTFYDDVKKR
RILWGWVNESSPAKDDIEKGWSGLQSFPRKIWLDESGKELLQWPIEEIETLRGQQVNWQKKVLKAGSTLQVHGVT
AAQADVEVSFKVKELEKADVIEPSWTDPQKICSQGDLSVMSGLGPFGLMVLASNDMEEYTSVYFRIFKSNDDTNK
KTKYVVLMCSDQSRSSLNDENDKSTFGAFVAIDPSHQTISLRTLIDHSIVESYGGGGRTCITSRVYPKLAIGENA
NLFVFNKGTQSVDILTLSAWSLKSAQINGDLMSPFIEREESRSPNHQF (SEQ ID NO: 25; secretion
signal is underlined)
DLNQPYRTGYHFQPLKNWMNGPMIYKGIYHLFYQYNPYGAVWDVRIVWGHSTSVDLVNWISQPPAFNPSQPSDIN
GCWSGSVTILPNGKPVILYTGIDQNKGQVQNVAVPVNISDPYLREWSKPPQNPLMTTNAVNGINPDRFRDPTTAW
LGRDGEWRVIVGSSTDDRRGLAILYKSRDFFNWTQSMKPLHYEDLTGMWECPDFFPVSITGSDGVETSSVGENGI
KHVLKVSLIETLHDYYTIGSYDREKDVYVPDLGFVQNESAPRLDYGKYYASKTFYDDVKKRRILWGWVNESSPAK
DDIEKGWSGLQSFPRKIWLDESGKELLQWPIEEIETLRGQQVNWQKKVLKAGSTLQVHGVTAAQADVEVSFKVKE
LEKADVIEPSWTDPQKICSQGDLSVMSGLGPFGLMVLASNDMEEYTSVYFRIFKSNDDTNKKTKYVVLMCSDQSR
SSLNDENDKSTFGAFVAIDPSHQTISLRTLIDHSIVESYGGGGRTCITSRVYPKLAIGENANLFVFNKGTQSVDI
LTLSAWSLKSAQINGDLMSPFIEREESRSPNHQF (SEQ ID NO: 26)
MASPSDLESPPTLSAQLLESRPPRSKLRLVALTLTAAAFLVALALFLADGSASRFVSGLARKLRSDPIKEHDYPW
TNEMLTWQRSGFHFQPAKNFQSDPNAAMYYKGWYHFFYQYNPTGTAWDYTISWGHAVSRDLIHWLHLPMAMVPDH
WYDAKGVWSGYSTLLPDGRVIVLYTGGTPELVQVQNLAVPADASDPLLLKWKKSSVNPILVPPPGIGTSDFRDPF
PIWYNETDSNWHVLIGSKDSNHHGIVLLYKTKDFFNFTLLPSLLHTSTQSVGMFECVDLYPVATGGPLSNRGLEM
SVDLSNGGIKHVLKASMDEERHDYYAIGTFDLDSFKWTPDDPSIDVGVGLRYDWGKFYASKTFFDTEKQRRILWG
YVGEVDSKDDDKMKGWATLQNIPRTILLDTKTQSNLIIWPVEEVEDLRTDGNIFNDIKIGAGSSVQLDIGAASQL
DIEAEFELDNSALDGAIEADVTYNCSTSGGAANRGLLGPFGLLVLANQDLTEQTATYFYVSRGTDGDLRTHFCQD
ELRSSKAGDIVKRVVGSVVPVLHGETWSLRILVDHSIIESFAQRGRAVATSRVYPTEAIYNKARLFLFNNATDAK
VTAKSVKIWHMNSTHNHPFPGLESLFES (SEQ ID NO: 4; secretion signal is underlined)
MRFPSIFTAVLFAASSALAAPVNTTTEDETAQIPAEAVIGYSDLEGDFDVAVLPFSNSTNNGLLFINTTIASIAA
KEEGVSLEKREAEARSDPIKEHDYPWTNEMLTWQRSGFHFQPAKNFQSDPNAAMYYKGWYHFFYQYNPTGTAWDY
TISWGHAVSRDLIHWLHLPMAMVPDHWYDAKGVWSGYSTLLPDGRVIVLYTGGTPELVQVQNLAVPADASDPLLL
KWKKSSVNPILVPPPGIGTSDFRDPFPIWYNETDSNWHVLIGSKDSNHHGIVLLYKTKDFFNFTLLPSLLHTSTQ
SVGMFECVDLYPVATGGPLSNRGLEMSVDLSNGGIKHVLKASMDEERHDYYAIGTFDLDSFKWTPDDPSIDVGVG
LRYDWGKFYASKTFFDTEKQRRILWGYVGEVDSKDDDKMKGWATLQNIPRTILLDTKTQSNLIIWPVEEVEDLRT
DGNIFNDIKIGAGSSVQLDIGAASQLDIEAEFELDNSALDGAIEADVTYNCSTSGGAANRGLLGPFGLLVLANQD
LTEQTATYFYVSRGTDGDLRTHFCQDELRSSKAGDIVKRVVGSVVPVLHGETWSLRILVDHSIIESFAQRGRAVA
TSRVYPTEAIYNKARLFLFNNATDAKVTAKSVKIWHMNSTHNHPFPGLESLFES (SEQ ID NO: 27;
secretion signal is underlined)
RSDPIKEHDYPWTNEMLTWQRSGFHFQPAKNFQSDPNAAMYYKGWYHFFYQYNPTGTAWDYTISWGHAVSRDLIH
WLHLPMAMVPDHWYDAKGVWSGYSTLLPDGRVIVLYTGGTPELVQVQNLAVPADASDPLLLKWKKSSVNPILVPP
PGIGTSDFRDPFPIWYNETDSNWHVLIGSKDSNHHGIVLLYKTKDFFNFTLLPSLLHTSTQSVGMFECVDLYPVA
TGGPLSNRGLEMSVDLSNGGIKHVLKASMDEERHDYYAIGTFDLDSFKWTPDDPSIDVGVGLRYDWGKFYASKTF
FDTEKQRRILWGYVGEVDSKDDDKMKGWATLQNIPRTILLDTKTQSNLIIWPVEEVEDLRTDGNIFNDIKIGAGS
SVQLDIGAASQLDIEAEFELDNSALDGAIEADVTYNCSTSGGAANRGLLGPFGLLVLANQDLTEQTATYFYVSRG
TDGDLRTHFCQDELRSSKAGDIVKRVVGSVVPVLHGETWSLRILVDHSIIESFAQRGRAVATSRVYPTEAIYNKA
RLFLFNNATDAKVTAKSVKIWHMNSTHNHPFPGLESLFES (SEQ ID NO: 28)
atgagatttccttcaatttttactgctgttttattcgcagcatcctccgcattagctgctccagtcaacactaca
acagaagatgaaacggcacaaattccggctgaagctgtcatcggttactcagatttagaaggggatttcgatgtt
gctgttttgccattttccaacagcacaaataacgggttattgtttataaatactactattgccagcattgctgct
aaagaagaaggggtatctctcgagaaaagagaggctgaagctaacttgatgcgtttaagagagaatgattatccc
tggactaacgacatgctaagatggcaacgcacgggatttcacttccagcctggtaaaaacttccaagccgaccca
aatgcagctatgttttacaagggctggtaccatttcttttatcaatacaacccgaccggtgtggcttgggattac
acaatctcctggggtcacgctgtcagtaaggatttgctgcattggaattatcttccaatggccttgaggcctgac

cactggtacgatagaaaaggtgtttggagcggttactctactttattgccagacggtagaattgttgtcttgtac
accggtggaactaaggaattagttcaagtccaaaacttggctgtcccagtaaacctttctgacccattgctattg
gaatggaagaagtcacacgttaacccaatactcgttccacctccggggatcgaagatcatgatttccgagatcca
ttcccagtgtggtataatgaatctgactcgcggtggcacgttgtaattggttccaaagatccagagcactatggt
attgtcttgatctacactaccaaggacttcgttaactttacgttattaccaaacatattgcattccaccaagcag
ccggttggtatgctggaatgtgtagacttgttcccagttgctacaactgattctcgtgcaaatcaagctttggat
atgactaccatgaggcccggtcctgggctcaaatatgtgttaaaggcgagtatggatgacgaaagacacgattac
tacgccctaggtagctttgacttggactcgttcacttttacaccagatgatgaaaccattgacgtcggtattggt
cttagatacgactggggcaagttctacgcgtccaagactttttacgaccaagaaaaacaaagaagagttttgtgg
ggatacgtcggtgaagttgactcgaagcgtgatgatgctctgaaaggttgggcttctttgcaaaatatcccacgt
acaatcttgttcgacaccaaaaccaagtccaacctaattttgtggccagttgaagaagtcgagtctttaagaact
attaacaagaatttcaattcaatccctttgtatcctggttctacttaccagcttgatgtgggtgaagctacccaa
ttggatattgtggccgagttcgaagtcgatgaaaaggctattgaagctactgccgaagctgatgttacatataac
tgctccacctccggtggtgcagctaatagaggggttttgggtccattcggtttgttagttttagctaaccaagag
ttgtctgaacaaactgctacttacttctatgtctctcgcggcatagatggtaacttaagaacacatttttgtcaa
gacgaactgcgatcttccaaggctggtgccatcactaagcgggtagttggttctaccgtcccagttctacatggc
gaaacctgggccttgagaattttggtcgatcactcaatcgtagagtcttttgcacagagaggtagagctgttgcc
acgagtagagtctatcctacagaagcaatttatagctcagctagagtctttctattcaacaatgccactgacgct
attgttaccgctaagacagtaaacgtttggcacatcaactccacctacaatcatgtttttccgggtctggtcgct
cca (SEQ ID NO: 5)
atgagatttccttcaatttttactgctgttttattcgcagcatcctccgcattagctgctccagtcaacactaca
acagaagatgaaacggcacaaattccggctgaagctgtcatcggttactcagatttagaaggggatttcgatgtt
gctgttttgccattttccaacagcacaaataacgggttattgtttataaatactactattgccagcattgctgct
aaagaagaaggggtatctctcgagaaaagagaggctgaagctaacttgatgcgtttaagagagaatgattatccc
tggactaacgacatgctaagatggcaacgcacgggatttcacttccagcctggtaaaaacttccaagccgaccca
aatgcagctatgttttacaagggctggtaccatttcttttatcaatacaacccgaccggtgtggcttgggattac
acaatctcctggggtcacgctgtcagtaaggatttgctgcattggaattatcttccaatggccttgaggcctgac
cactggtacgatagaaaaggtgtttggagcggttactctactttattgccagacggtagaattgttgtcttgtac
accggtggaactaaggaattagttcaagtccaaaacttggctgtcccagtaaacctttctgacccattgctattg
gaatggaagaagtcacacgttaacccaatactcgttccacctccggggatcgaagatcatgatttccgagatcca
ttcccagtgtggtataatgaatctgactcgcggtggcacgttgtaattggttccaaagatccagagcactatggt
attgtcttgatctacactaccaaggacttcgttaactttacgttattaccaaacatattgcattccaccaagcag
ccggttggtatgctggaatgtgtagacttgttcccagttgctacaactgattctcgtgcaaatcaagctttggat
atgactaccatgaggcccggtcctgggctcaaatatgtgttaaaggcgagtatggatgacgaaagacacgattac
tacgccctaggtagctttgacttggactcgttcacttttacaccagatgatgaaaccattgacgtcggtattggt
cttagatacgactggggcaagttctacgcgtccaagactttttacgaccaagaaaaacaaagaagagttttgtgg
ggatacgtcggtgaagttgactcgaagcgtgatgatgctctgaaaggttgggcttctttgcaaaatatcccacgt
acaatcttgttcgacaccaaaaccaagtccaacctaattttgtggccagttgaagaagtcgagtctttaagaact
attaacaagaatttcaattcaatccctttgtatcctggttctacttaccagcttgatgtgggtgaagctacccaa
ttggatattgtggccgagttcgaagtcgatgaaaaggctattgaagctactgccgaagctgatgttacatataac
tgctccacctccggtggtgcagctaatagaggggttttgggtccattcggtttgttagttttagctaaccaagag
ttgtctgaacaaactgctacttacttctatgtctctcgcggcatagatggtaacttaagaacacatttttgtcaa
gacgaactgcgatcttccaaggctggtgccatcactaagcgggtagttggttctaccgtcccagttctacatggc
gaaacctgggccttgagaattttggtcgatcactcaatcgtagagtcttttgcacagagaggtagagctgttgcc
acgagtagagtctatcctacagaagcaatttatagctcagctagagtctttctattcaacaatgccactgacgct
attgttaccgctaagacagtaaacgtttggcacatcaactccacctacaatcatgtttttccgggtctggtcgct
ccataa (SEQ ID NO: 62)
atgagatttccttcaatttttactgctgttttattcgcagcatcctccgcattagctgctccagtcaacactaca
acagaagatgaaacggcacaaattccggctgaagctgtcatcggttactcagatttagaaggggatttcgatgtt
gctgttttgccattttccaacagcacaaataacgggttattgtttataaatactactattgccagcattgctgct
aaagaagaaggggtatctctcgagaaaagagaggctgaagctgacttgaatcaaccttatagaaccggttaccac
ttccagccattaaaaaactggatgaacggcccaatgatttacaagggaatctatcatctgttttaccaatacaac
ccatacggtgccgtgtgggatgtaaggattgtctggggtcacagtacttccgtcgatttggttaattggataagc
caacccccggcattcaacccatcacaaccatctgacatcaacggttgttggtcgggttctgttacgattctacct
aatgggaagccagttatcctttatacaggtattgatcaaaacaagggtcaagttcagaatgtcgcggttccagtc
aatatctctgacccatatttgcgtgaatggtccaaaccacctcaaaacccattgatgactaccaacgctgttaac
ggtatcaaccctgatagatttagagatccaactacagcttggctaggaagagatggtgagtggagagtcattgtg
ggttcatctaccgacgaccgccggggtttggccatattatacaagtcccgcgatttctttaattggactcaatct
atgaaaccgttgcattacgaagatttgaccggaatgtgggaatgcccagacttcttcccagtttcaattacgggg

agtgatggtgtggaaacttcttccgtaggtgaaaacggtataaagcacgttctcaaggtcagcttaatcgaaact
ttgcatgactactataccattggttcgtatgacagagagaaggatgtctacgttcctgacttaggtttcgtccaa
aatgaatccgctccacgtttggattacgggaaatactacgcctctaagacattttatgacgacgtcaaaaagcgg
agaattttatggggttgggttaacgaatcttcgccagctaaggacgatattgaaaagggctggtctggtttgcag
tcatttccaagaaagatttggttggacgagagcggtaaagaattgctgcaatggccaatcgaagaaatagaaact
ctacgtggccaacaagttaactggcaaaagaaggttttgaaggctggttctaccttacaagtccacggtgttact
gctgctcaagcggatgtagaggtttccttcaaagtcaaggaattggaaaaagcagacgtcatcgaaccctcctgg
accgatccccaaaaaatatgttcgcagggtgacttgtctgttatgtctggtttaggtccgttcggtcttatggtt
cttgcttctaatgatatggaagaatacacttccgtttacttcagaatcttcaagagtaacgatgatactaataaa
aagaccaagtatgttgtgctcatgtgttccgatcaatcaagaagttctttgaacgatgagaacgataagtcaacc
tttggggcctttgttgctattgatccatctcatcagaccatctctctccgaacattgattgaccactccatagtc
gaatcatacggtggtggtggcagaacttgtatcacgagtagagtatatccaaagttggccatcggtgaaaatgca
aatttattcgtctttaacaagggtactcaatctgttgacattctgactttaagcgcttggtcccttaagagtgct
caaattaacggagacttgatgtctcctttcatcgagagagaagaaagtagatcacccaaccatcaattctaa
(SEQ ID NO: 29)
atgagatttccttcaatttttactgctgttttattcgcagcatcctccgcattagctgctccagtcaacactaca
acagaagatgaaacggcacaaattccggctgaagctgtcatcggttactcagatttagaaggggatttcgatgtt
gctgttttgccattttccaacagcacaaataacgggttattgtttataaatactactattgccagcattgctgct
aaagaagaaggggtatctctcgagaaaagagaggctgaagctagatcagatcctattaaagagcatgactatcca
tggactaatgaaatgttgacatggcaacgtagtggatttcacttccagcccgctaagaacttccaatccgaccca
aacgcagccatgtactacaagggctggtatcacttcttttaccaatacaatccgaccggtactgcttgggattac
acgatctcttggggtcatgctgtctcgcgggacttaatacactggcttcatctgccaatggctatggtaccagat
cactggtatgatgcgaagggtgtgtggtccggttactctaccctattgccagatggtagagttattgtcttatat
actggtggtaccccagaattggttcaagttcaaaacttggccgttcctgctgacgcctctgatccactgttgttg
aaatggaagaagtcctcagtcaaccccatccttgttccgccaccagggattggaactagcgacttcagggatcca
tttcctatctggtacaatgaaacagactccaactggcacgtcttgataggttctaaagactccaaccaccatggt
attgtattattgtataagactaaggacttctttaacttcacattgcttccatctttattgcacaccagtacccag
agcgttggtatgttcgaatgcgtggatctctacccagtcgctactggtgggccactatctaatagaggtttggaa
atgagcgttgatctctcaaatggtggtatcaaacatgttttgaaggcttctatggatgaggaaagacatgactac
tatgcgattggcacctttgacttagattctttcaaatggacgcccgacgatccaagtatcgacgttggtgtcggt
ctaagatacgattggggtaagttctacgcttctaagaccttttttgatactgaaaagcaacgccgaattttatgg
ggctatgtcggtgaagttgactccaaggatgatgacaagatgaaaggttgggcaaccttacaaaatatacctaga
actatcttgcttgacacgaaaactcaatctaacttgattatctggccagtcgaggaagttgaagatttgagaact
gacggcaacattttcaacgatataaaaattggtgctggttcttcagtacaattggatattggtgccgcttcgcag
ttggacatcgaagccgaatttgaactagataacagtgctttggacggcgctattgaagctgatgtcacttacaat
tgttcaacttcgggtggtgccgcaaatagaggtttgctggggcctttcggtttacttgttttagctaaccaagac
ttgacagaacaaaccgctacatacttctacgtgtccagaggtaccgatggtgatttgagaacccacttctgtcaa
gacgaattacgttcctccaaggcaggagacattgtcaagcgcgttgttggttctgtggtgccagttctacatggt
gaaacttggtccttgagaattttggttgaccactctatcatcgaaagctttgcacaaagaggacgggctgttgct
acctctagggtctacccaactgaggcaatctacaacaaagccagactgtttttgttcaacaatgctacagacgct
aaggttactgccaagagtgttaaaatatggcatatgaactctacacacaaccatccattccctggtttagaatcg
ctattcgaatcataa (SEQ ID NO: 30)
1-SST from Festuca arundinacea:
MESSAVVPGTTAPLLPYAYAPLPSSADDARENQSSGGVRWRVCAAVLAASALAVLIVVGLLAGGRVDRGPAGGDV
ASAAVPAVPMEIPRSRGKDFGVSEKASGAYSADGGFPWSNAMLQWQRTGFHFQPEKHYMNDPNGPVYYGGWYHLF
YQYNPKGDSWGNIAWAHAVSKDMVNWRHLPLAMVPDQWYDSNGVLTGSITVLPDGQVILLYTGNTDTLAQVQCLA
TPADPSDPLLREWIKHPANPILYPPPGIGLKDFRDPLTAWFDHSDNTWRTVIGSKDDDGHAGIILSYKTKDFVNY
ELMPGNMHRGPDGTGMYECIDLYPVGGNSSEMLGGDDSPDVLFVLKESSDDERHDYYALGRFDAAANIWTPIDQE
LDLGIGLRYDWGKYYASKSFYDQKKNRRIVWAYIGETDSEQADITKGWANLMTIPRTVELDKKTRTNLIQWPVEE
LDTLRRNSTDLSGITVDAGSVIRLPLHQGAQIDIEASFQLNSSDVDALTEADVSYNCSTSGAAVRGALGPFGLLV
LANGRTEQTAVYFYVSKGVDGALQTHFCHDESRSTQAKDVVNRMIGSIVPVLDGETFSVRVLVDHSIVQSFAMGG
RITATSRAYPTEAIYAAAGVYLFNNATGATVTAERLVVYEMASADNHIFTNDDL (SEQ ID NO: 6)
Non-limiting examples of 1-FFT sequences
MRFPSIFTAVLFAASSALAAPVNTTTEDETAQIPAEAVIGYSDLEGDFDVAVLPFSNSTNNGLLFINTTIASIAA
KEEGVSLEKREAEASSVQPSAAERLTWERTAFHFQPAKNFIYDPNGPLFHMGWHHLFYQYNPYAPVWGNMSWGHA
VSKDMINWFELPVALVPTEWYDIEGVLSGSTTALPNGQIFALYTGNANDFSQLQCKAVPVDVSDPLLVKWVKYDG
NPILYTPPGIGLKDYRDPSTVWTGPDGKHRMIMGTKRGTTGLVLVYHTTDFTNYVMLDEPLHSVPNTDMWECVDL
FPVSTTNDSALDIAAYGSGIKHVLKESWEGHAMDFYSIGTYDAINDKWTPDNPELDVGIGLRCDYGRFFASKSLY
DPLKKRRVTWGYVAESDSADQDVSRGWATIYNVARTIVLDRKTGTHLLQWPVEELESLRSNVREFKEMTLEPGSI
VPLDIGSATQLDIIATFEVDQEALKATSDANDEYACTTSSGAAERGSFGPFGIAVLADGTLSELTPVYFYIAKNT
KGGVDTHFCTDKLRSSLDYDSEKVVYGSTIPVLDGEQITMRVLVDHSVVEGFAQGGRTVITSRVYPTKAIYEGAK
LFVFNNATTTNVKATLNVWQMSHALIQPYPF (SEQ ID NO: 7; secretion signal is
underlined)
MKTTEPLTDLEHAPNHTPLLDHPQPPPATVSKRLLIRVLSSITFVSLFFVSAFLLILLNQHESSYTDDNLAPLDR
SSVQPSAAERLTWERTAFHFQPAKNFIYDPNGPLFHMGWHHLFYQYNPYAPVWGNMSWGHAVSKDMINWFELPVA
LVPTEWYDIEGVLSGSTTALPNGQIFALYTGNANDFSQLQCKAVPVDVSDPLLVKWVKYDGNPILYTPPGIGLKD
YRDPSTVWTGPDGKHRMIMGTKRGTTGLVLVYHTTDFTNYVMLDEPLHSVPNTDMWECVDLFPVSTTNDSALDIA
AYGSGIKHVLKESWEGHAMDFYSIGTYDAINDKWTPDNPELDVGIGLRCDYGRFFASKSLYDPLKKRRVTWGYVA
ESDSADQDVSRGWATIYNVARTIVLDRKTGTHLLQWPVEELESLRSNVREFKEMTLEPGSIVPLDIGSATQLDI1
ATFEVDQEALKATSDANDEYACTTSSGAAERGSFGPFGIAVLADGTLSELTPVYFYIAKNTKGGVDTHFCTDKLR
SSLDYDSEKVVYGSTIPVLDGEQITMRVLVDHSVVEGFAQGGRTVITSRVYPTKAIYEGAKLFVFNNATTTNVKA
TLNVWQMSHALIQPYPF (SEQ ID NO: 8; secretion signal is underlined)
SSVQPSAAERLTWERTAFHFQPAKNFIYDPNGPLFHMGWHHLFYQYNPYAPVWGNMSWGHAVSKDMINWFELPVA
LVPTEWYDIEGVLSGSTTALPNGQIFALYTGNANDFSQLQCKAVPVDVSDPLLVKWVKYDGNPILYTPPGIGLKD
YRDPSTVWTGPDGKHRMIMGTKRGTTGLVLVYHTTDFTNYVMLDEPLHSVPNTDMWECVDLFPVSTTNDSALDIA
AYGSGIKHVLKESWEGHAMDFYSIGTYDAINDKWTPDNPELDVGIGLRCDYGRFFASKSLYDPLKKRRVTWGYVA
ESDSADQDVSRGWATIYNVARTIVLDRKTGTHLLQWPVEELESLRSNVREFKEMTLEPGSIVPLDIGSATQLDI1
ATFEVDQEALKATSDANDEYACTTSSGAAERGSFGPFGIAVLADGTLSELTPVYFYIAKNTKGGVDTHFCTDKLR
SSLDYDSEKVVYGSTIPVLDGEQITMRVLVDHSVVEGFAQGGRTVITSRVYPTKAIYEGAKLFVFNNATTTNVKA
TLNVWQMSHALIQPYPF (SEQ ID NO: 31)
MKTIEPFSDVENAPNSTPLLNHPEPPRAAVRKQSFVRVLSSITLVSLFFVLAFVLIVLNQQDSTTTVANSAPPGA
TVPEKSSVKHSQSDRLRWERTAYHFQPAKNFIYDPNGPLFHMGWYHLFYQYNPYAPIWGNMSWGHAVSKDMIHWF
ELPVAIVPTEWYDIEGVLSGSTTALPNGQIFALYTGNAKDFSQLQCKAVPLNASDPLLVEWVKYEDNPILYIPPG
IGPKDYRDPSTVWTGPDGKHRMIMGTKQNGTGMVHVYHTTDFINYVLLDEPLHSVPNTDMWECVDFYPVSTINDS
ALDIAAYGSDIKHVIKESWEGHGMDLYSIGTYDAYKDKWTPDNPEFDVGIGLRVDYGRFFASKSLYDPLKKRRVT
WGYVAESDSSDQDLNRGWATIYNVGRTVVLDRKTGTHLLHWPVEEIESLRSNVREFNEIELVPGSIIPLDIGMAT
QLDIVATFKVDPEALMAKSDINSEYGCTTSSGATQRGSLGPFGIVVLADVALSELTPVYFYIAKNIDGGLVTHFC
TDKLRSSLDYDGERVVYGSTVPVLDGEELTMRLLVDHSVVEGFAQGGRTVMTSRVYPTNAIYEEAKIFLFNNATG
ASVKASLKIWQMGSASIQAYPF (SEQ ID NO: 9; secretion signal is underlined)
MRFPSIFTAVLFAASSALAAPVNTTTEDETAQIPAEAVIGYSDLEGDFDVAVLPFSNSTNNGLLFINTTIASIAA
KEEGVSLEKREAEASSVKHSQSDRLRWERTAYHFQPAKNFIYDPNGPLFHMGWYHLFYQYNPYAPIWGNMSWGHA
VSKDMIHWFELPVAIVPTEWYDIEGVLSGSTTALPNGQIFALYTGNAKDFSQLQCKAVPLNASDPLLVEWVKYED
NPILYIPPGIGPKDYRDPSTVWTGPDGKHRMIMGTKQNGTGMVHVYHTTDFINYVLLDEPLHSVPNTDMWECVDF
YPVSTINDSALDIAAYGSDIKHVIKESWEGHGMDLYSIGTYDAYKDKWTPDNPEFDVGIGLRVDYGRFFASKSLY
DPLKKRRVTWGYVAESDSSDQDLNRGWATIYNVGRTVVLDRKTGTHLLHWPVEEIESLRSNVREFNEIELVPGSI
IPLDIGMATQLDIVATFKVDPEALMAKSDINSEYGCTTSSGATQRGSLGPFGIVVLADVALSELTPVYFYIAKNI
DGGLVTHFCTDKLRSSLDYDGERVVYGSTVPVLDGEELTMRLLVDHSVVEGFAQGGRTVMTSRVYPTNAIYEEAK
IFLFNNATGASVKASLKIWQMGSASIQAYPF (SEQ ID NO: 32; secretion signal is
underlined)
SSVKHSQSDRLRWERTAYHFQPAKNFIYDPNGPLFHMGWYHLFYQYNPYAPIWGNMSWGHAVSKDMIHWFELPVA
IVPTEWYDIEGVLSGSTTALPNGQIFALYTGNAKDFSQLQCKAVPLNASDPLLVEWVKYEDNPILYIPPGIGPKD
YRDPSTVWTGPDGKHRMIMGTKQNGTGMVHVYHTTDFINYVLLDEPLHSVPNTDMWECVDFYPVSTINDSALDIA
AYGSDIKHVIKESWEGHGMDLYSIGTYDAYKDKWTPDNPEFDVGIGLRVDYGRFFASKSLYDPLKKRRVTWGYVA
ESDSSDQDLNRGWATIYNVGRTVVLDRKTGTHLLHWPVEEIESLRSNVREFNEIELVPGSIIPLDIGMATQLDIV
ATFKVDPEALMAKSDINSEYGCTTSSGATQRGSLGPFGIVVLADVALSELTPVYFYIAKNIDGGLVTHFCTDKLR
SSLDYDGERVVYGSTVPVLDGEELTMRLLVDHSVVEGFAQGGRTVMTSRVYPTNAIYEEAKIFLFNNATGASVKA
SLKIWQMGSASIQAYPF (SEQ ID NO: 33)
MKTIEPFSDVENAPNSTPLLNHPEPSRAAVRKQSFVRVLSSITLVSLFFVLAFVLIVLNQQDSTNTVANSAPPGA
TVPEKSSVKHSQSDRLRWERTAYHFQPAKNFIYDPNGPLFHMGWYHLFYQYNPYAPIWGNMSWGHAVSKDMIHWF
ELPVAMVPTEWYDIEGVLSGSTTALPNGQIFALYTGNAKDFSQLQCKAVPLNASDPLLVDWVKYEDNPILYIPPG
IGPKDYRDPSTVWTGPDGKHRMIMGTKQNGTGMVHVYHTTDFINYVLLDEPLHSVPNTDMWECVDFYPVSTINDS
ALDIAAYGSDIKHVIKESWEGHGMDLYSIGTYDAYKDKWTPDNPELDVGIGLRVDYGRLFASKSLYDPLKKRRVT
WGYVGESDSPDQDINRGWATIYNVGRTWLDRKTGTHLLHWPVEEIESLRSNVREFNEIELVPGSIIPLDIGMAT
QLDIVATFKVDPEALMAKSDINSEYGCTTSSGATQRGSLGPFGIVVLADLALSELTPLYFYIAKNTDGGLVTHFC
TDKLRSSLDYDGERVVYGGTVPVLDGEELTMRLLVDHSVVEGFAQGGRTVITSRVYPTNAIYEEAKIFLFNNATG
ASVKASLKIWQMGSASIQAYPF (SEQ ID NO: 10; secretion signal is underlined)
MRFPSIFTAVLFAASSALAAPVNTTTEDETAQIPAEAVIGYSDLEGDFDVAVLPFSNSTNNGLLFINTTIASIAA
KEEGVSLEKREAEASSVKHSQSDRLRWERTAYHFQPAKNFIYDPNGPLFHMGWYHLFYQYNPYAPIWGNMSWGHA
VSKDMIHWFELPVAMVPTEWYDIEGVLSGSTTALPNGQIFALYTGNAKDFSQLQCKAVPLNASDPLLVDWVKYED
NPILYIPPGIGPKDYRDPSTVWTGPDGKHRMIMGTKQNGTGMVHVYHTTDFINYVLLDEPLHSVPNTDMWECVDF
YPVSTINDSALDIAAYGSDIKHVIKESWEGHGMDLYSIGTYDAYKDKWTPDNPELDVGIGLRVDYGRLFASKSLY
DPLKKRRVTWGYVGESDSPDQDINRGWATIYNVGRTVVLDRKTGTHLLHWPVEEIESLRSNVREFNEIELVPGSI
IPLDIGMATQLDIVATFKVDPEALMAKSDINSEYGCTTSSGATQRGSLGPFGIVVLADLALSELTPLYFYIAKNT
DGGLVTHFCTDKLRSSLDYDGERVVYGGTVPVLDGEELTMRLLVDHSVVEGFAQGGRTVITSRVYPTNAIYEEAK
IFLFNNATGASVKASLKIWQMGSASIQAYPF (SEQ ID NO: 34; secretion signal is
underlined)
SSVKHSQSDRLRWERTAYHFQPAKNFIYDPNGPLFHMGWYHLFYQYNPYAPIWGNMSWGHAVSKDMIHWFELPVA
MVPTEWYDIEGVLSGSTTALPNGQIFALYTGNAKDFSQLQCKAVPLNASDPLLVDWVKYEDNPILYIPPGIGPKD
YRDPSTVWTGPDGKHRMIMGTKQNGTGMVHVYHTTDFINYVLLDEPLHSVPNTDMWECVDFYPVSTINDSALDIA
AYGSDIKHVIKESWEGHGMDLYSIGTYDAYKDKWTPDNPELDVGIGLRVDYGRLFASKSLYDPLKKRRVTWGYVG
ESDSPDQDINRGWATIYNVGRTVVLDRKTGTHLLHWPVEEIESLRSNVREFNEIELVPGSIIPLDIGMATQLDIV
ATFKVDPEALMAKSDINSEYGCTTSSGATQRGSLGPFGIVVLADLALSELTPLYFYIAKNTDGGLVTHFCTDKLR
SSLDYDGERVVYGGTVPVLDGEELTMRLLVDHSVVEGFAQGGRTVITSRVYPTNAIYEEAKIFLFNNATGASVKA
SLKIWQMGSASIQAYPF (SEQ ID NO: 35)
atgagatttccttcaatttttactgctgttttattcgcagcatcctccgcattagctgctccagtcaacactaca
acagaagatgaaacggcacaaattccggctgaagctgtcatcggttactcagatttagaaggggatttcgatgtt
gctgttttgccattttccaacagcacaaataacgggttattgtttataaatactactattgccagcattgctgct
aaagaagaaggggtatctctcgagaaaagagaggctgaagctagttccgttcaaccttctgccgctgaacgttta
acctgggagagaactgcattccattttcagccagctaaaaatttcatttatgatccaaacggaccgctgtttcac
atgggctggcaccatcttttctaccaatacaacccctacgctccagtctggggtaatatgagctggggtcacgcg
gtgtcaaaggacatgataaactggttcgaattgccagtagccttagttccaacggaatggtatgatattgaaggt
gttctatctggttctactacagctttgcctaatgggcaaatctttgctttgtacaccggtaacgccaacgacttc
tcccaattgcaatgtaaggctgtcccagttgacgtgtcggatccattattggtcaaatgggttaagtatgacggt
aatccgatcttgtacactccacctggaatcggtctgaaggattatagagatccatctaccgtctggactggtcca
gacggtaagcataggatgattatgggtacaaagagaggtaccactggcttggttttagtttaccacacaacggat
ttcactaactacgtcatgttggacgaaccactccactcagtaccaaacactgacatgtgggaatgcgttgatctt
tttccggtcagcaccaccaatgatagtgctttggacatcgcggcttatggttccggtattaaacatgttttgaaa
gagtcttgggaaggtcacgcaatggatttctactccattgggacttacgatgctataaacgacaagtggactcct
gacaacccagaactagacgtcggtattggtttgagatgtgattacggtagatttttcgcatctaagtccctatac
gatcctttaaagaaacggagagttacctggggatatgtcgccgaatctgattcagccgaccaagacgtgtctcgc
ggttgggctacaatctataatgttgcaaggactattgttttagaccgtaagaccggcactcatctgcttcagtgg
ccagtcgaagaattggagtcccttagatcgaacgtgagagaatttaaggaaatgaccttggaaccaggttccatc
gttccattggatataggttctgctactcaattggatattatcgctacgttcgaagttgaccaagaagctttgaaa
gctacctctgacgctaacgacgaatacgcctgtacaacatcttcaggtgctgcggagcgtggttcgttcggtccc
ttcggtatcgctgtcctcgccgatggtaccttgtccgaactgactccagtatacttctacattgctaaaaatact
aagggcggggtcgatacgcacttttgtactgataagttgagaagctctttagactatgacagtgaaaaggttgtc
tacgggagtaccattccagttttagatggtgaacaaatcactatgagagttctcgtcgatcattccgttgtggaa
ggttttgcccagggtggtagaactgtaattaccagtagagtttaccctaccaaggctatatacgaaggtgccaag
ttgtttgtattcaataacgctacaactacaaatgttaaggcaacgttgaatgtatggcaaatgtcacacgccctc
atccaaccatacccattctaa (SEQ ID NO: 11)
atgagatttccttcaatttttactgctgttttattcgcagcatcctccgcattagctgctccagtcaacactaca
acagaagatgaaacggcacaaattccggctgaagctgtcatcggttactcagatttagaaggggatttcgatgtt
gctgttttgccattttccaacagcacaaataacgggttattgtttataaatactactattgccagcattgctgct
aaagaagaaggggtatctctcgagaaaagagaggctgaagctagttccgttaaacattctcagtcagatcgattg
aggtgggaacgtactgcctaccactttcaaccagcaaagaacttcatatatgaccctaatggtccacttttccac
atgggatggtaccatctattttaccaatataacccgtatgctccaatttggggcaatatgtcttggggtcacgct
gtgtccaaggacatgatccattggttcgagctgcccgtcgctatcgttccaacggaatggtacgatattgaaggt
gtattaagcggttcgacaactgcgttgccaaacggtcaaattttcgccttgtacaccggtaatgctaaggatttt
tctcaattacaatgcaaagctgtccctttgaacgcttccgacccattgttggttgaatgggttaagtacgaagat
aaccctatcctatatattccaccaggcatcggtcctaaggactacagagatccatctaccgtgtggacaggtcca
gatggtaaacacagaatgattatgggaaccaagcaaaacggtactgggatggttcatgtctaccacaccactgac
tttataaattatgtcttattagacgagccgttgcactccgtcccaaacaccgatatgtgggaatgtgtggacttc
tacccagtatctactatcaatgacagcgcgttggatattgcagcctacggttcagacatcaagcatgttataaaa
gaatcttgggaaggtcatggtatggatttatactctattggtacttatgacgcttacaaggataagtggacgcca
gataaccccgagttcgatgttgggattggtctgagagttgattacggcagattctttgcttccaagagcttgtac
gacccgttgaagaagagaagagtcacatggggttatgttgctgaaagtgattcttccgaccaagacctcaataga
ggttgggccacaatctataacgttggtagaactgtcgtcttggaccggaaaaccggtacacacctattacattgg
ccagtggaggaaattgaatctctgcgttcgaacgtcagagaatttaatgaaattgaattggttccaggatcgatc
ataccattggatattggtatggctactcaattggacatcgttgccaccttcaaagtagacccagaagctcttatg
gctaagtccgatattaactctgaatacggttgtaccacttcctcaggtgctactcagcgtgggtctttaggccct
tttggtatcgttgttttggctgacgtagctctatcggagttaaccccagtttacttctatatcgcaaagaatatc
gatggtggtctggtcactcacttctgtaccgataaattgcgctctagtttggactacgatggagaaagagttgtt
tacggttcaactgttccagtcttggacggtgaagaattaaccatgagattgctggtggatcatagtgtagtcgaa
ggtttcgctcaaggtggtagaactgttatgacctccagagtctaccccactaacgccatctatgaagaggcgaag
atttttcttttcaataacgcgactggcgctagtgttaaagcatctttgaagatttggcaaatgggttctgcctct
attcaggcttatcccttctaa (SEQ ID NO: 36)
atgagatttccttcaatttttactgctgttttattcgcagcatcctccgcattagctgctccagtcaacactaca
acagaagatgaaacggcacaaattccggctgaagctgtcatcggttactcagatttagaaggggatttcgatgtt
gctgttttgccattttccaacagcacaaataacgggttattgtttataaatactactattgccagcattgctgct
aaagaagaaggggtatctctcgagaaaagagaggctgaagctagttccgttaaacattctcagtcagatcgattg
aggtgggaacgtactgcctaccactttcaaccagcaaagaacttcatatatgaccctaatggtccacttttccac
atgggatggtaccatctattttaccaatataacccgtatgctccaatttggggcaatatgtcttggggtcacgct
gtgtccaaggacatgatccattggttcgagctgcccgtcgctatggttccaacggaatggtacgatattgaaggt
gtcttgtctgggagcaccacagctttgcctaacggtcaaatcttcgccttatacactggtaatgcgaaagatttt
tcccaattacaatgcaaggctgttccattgaacgcctcggacccattgctcgtagattgggtcaagtacgaagat
aacccaattttgtatatccccccaggtattggaccaaaggactacagagatccgagtaccgtgtggactggtcct
gacggtaaacacagaatgatcatgggtaccaagcaaaacggcactggtatggttcacgtataccatacaaccgac
tttattaattatgttttattggacgaaccattgcactctgttccaaatactgatatgtgggagtgtgtcgatttc
tacccagtctctacgataaacgacagcgcactcgatatagctgcttatggtagtgatattaagcacgttattaaa
gaatcttgggaaggtcatggtatggacttgtactccatcggtacttacgatgcttacaaggataagtggacccca
gacaaccctgaattagacgttggtatcgggctaagagtggactatggtagattgttcgcatcgaaaagcctttac
gatccactgaagaaaagaagagtcacttggggttacgttggcgagtctgattctccagatcaggacattaacaga
ggttgggcgaccatctataatgttggacgtaccgtcgttttggatagaaagactggtactcatctactgcactgg
cctgtcgaagaaatcgaatcattaagaagtaatgttagagaatttaacgaaattgagttggtaccaggttctata
attcctttggacattggtatggccacacaattggacatcgttgctacattcaaggttgatccagaagctttaatg
gctaagtctgacataaactccgaatacggttgtaccacttcctccggtgcgactcaaagaggttcgttgggtcca
ttcggtatcgtcgttctagccgatttggctctctctgaattgactccattatacttttatatcgctaagaacacc
gatgggggcttggtaacacacttctgtactgataaattaagatcaagtttggattacgacggtgaacgcgtcgta
tacggtggtacggttcccgtgttagacggggaagaactcaccatgaggctattggtcgatcattctgttgttgag
ggttttgctcaaggtggaagaaccgttattactagccgtgtctatcccacaaatgctatttatgaagaagccaag
attttcctttttaacaacgctaccggtgcatccgttaaggcttctttgaagatatggcaaatgggtagcgcttct
atccaagcctacccattctaa (SEQ ID NO: 37)
1-FFT from Echinops ritro:
EPFSDLEHAPNHTPLLDRPKTPPAAVSHRLLIRVLSTITVVSLFFVAAFLLVLNQQDSGNNPLPQDPPPQPSAAD
RLRWERTAYHYQPAKNFMYDPNGPIFHMGWYHLFYQYNPYSVFWGNMTWGHAVSKDMINWFELPVALAPVEWYDI
EGVLSGSTTVLPTGEIFALYTGNANDFSQLQCKAVPVNTSDPLLIDWVRYEGNPILYTPPGVGLTDYRDPSTVWT
GPDNIHRMIIGTRRNNTGLVLVYHTKDFINYELLDEPLHSVPDSGMWECVDLYPVSTMNDTALDVAAYGSGIKHV
LKESWEGHAKDFYSIGTYDAINDKWWPDNPELDLGMGWRCDYGRFFASKTLYDPLKKRRVTWGYVAESDSGDQDR
SRGWSNIYNVARTVMLDRKTGTNLLQWPVEEIESLRSKVHEFNEIELQPGSIIPLEVGSTTQLDIVATFEVNKDA
FEETNVNYNEYGCTSSKGASQRGRLGPFGIIVLADGNLLELTPVYFYIAKNNDGSLTTHFCTDKLRSSFDYDDEK
VVYGSTVPVLEGEKLTIRLMVDHSIIEGFAQGGRTVITSRVYPTKAIYDTAKLFLFNNATDITVKASLKVWHMAS
ANIQMYPF (SEQ ID NO: 12)
Non-limiting examples of 6-SFT sequences
MRFPSIFTAVLFAASSALAAPVNTTTEDETAQIPAEAVIGYSDLEGDFDVAVLPFSNSTNNGLLFINTTIASIAA
KEEGVSLEKREAEAVPGKLESNADVEWQRSAYHFQPDKNFISDPDGPMYHMGWYHLFYQYNPESAIWGNITWGHS
VSRDMINWFHLPFAMVPDHWYDIEGVMTGSATVLPNGQIIMLYTGNAYDLSQLQCLAYAVNSSDPLLLEWKKYEG
NPILFPPPGVGYKDFRDPSTLWMGPDGEWRMVMGSKHNETIGCALVYRTTNFTHFELNEEVLHAVPHTGMWECVD
LYPVSTTHTNGLEMKDNGPNVKYILKQSGDEDRHDWYAIGTFDPEKDKWYPDDPENDVGIGLRYDYGKFYASKTF
YDQHKKRRVLWGYVGETDPPKSDLLKGWANILNIPRSVVLDTQTETNLIQWPIEEVEKLRSKKYDEFKDVELRPG
SLIPLEIGTATQLDISATFEIDEKKLESTLEADVLFNCTTSEGSVGRGVLGPFGIVVLADANRSEQLPVYFYIAK
DTDGTSRTYFCADESRSSKDKDVGKWVYGSSVPVLEGENYNMRLLVDHSIVEGFAQGGRTVVTSRVYPTMAIYGA
AKIFLFNNATGISVKASLKIWKMAEAQLDPFPLSGWSS (SEQ ID NO: 13; secretion signal is
underlined)
MASSTTATTPLILRDETQIRPQLAGSSVGRRLSMAKILSGILVFVLVICALVAVIHDQSQQTMATNNHQGGDKPT
SAATFTAPLPQVGLKRVPGKLESNADVEWQRSAYHFQPDKNFISDPDGPMYHMGWYHLFYQYNPESAIWGNITWG
HSVSRDMINWFHLPFAMVPDHWYDIEGVMTGSATVLPNGQIIMLYTGNAYDLSQLQCLAYAVNSSDPLLLEWKKY
EGNPILFPPPGVGYKDFRDPSTLWMGPDGEWRMVMGSKHNETIGCALVYRTTNFTHFELNEEVLHAVPHTGMWEC
VDLYPVSTTHTNGLEMKDNGPNVKYILKQSGDEDRHDWYAIGTFDPEKDKWYPDDPENDVGIGLRYDYGKFYASK
TFYDQHKKRRVLWGYVGETDPPKSDLLKGWANILNIPRSVVLDTQTETNLIQWPIEEVEKLRSKKYDEFKDVELR
PGSLIPLEIGTATQLDISATFEIDEKKLESTLEADVLFNCTTSEGSVGRGVLGPFGIVVLADANRSEQLPVYFYI
AKDTDGTSRTYFCADESRSSKDKDVGKWVYGSSVPVLEGENYNMRLLVDHSIVEGFAQGGRTVVTSRVYPTMAIY
GAAKIFLFNNATGISVKASLKIWKMAEAQLDPFPLSGWSS (SEQ ID NO: 14; secretion signal
is underlined)
VPGKLESNADVEWQRSAYHFQPDKNFISDPDGPMYHMGWYHLFYQYNPESAIWGNITWGHSVSRDMINWFHLPFA
MVPDHWYDIEGVMTGSATVLPNGQIIMLYTGNAYDLSQLQCLAYAVNSSDPLLLEWKKYEGNPILFPPPGVGYKD
FRDPSTLWMGPDGEWRMVMGSKHNETIGCALVYRTTNFTHFELNEEVLHAVPHTGMWECVDLYPVSTTHTNGLEM
KDNGPNVKYILKQSGDEDRHDWYAIGTFDPEKDKWYPDDPENDVGIGLRYDYGKFYASKTFYDQHKKRRVLWGYV
GETDPPKSDLLKGWANILNIPRSVVLDTQTETNLIQWPIEEVEKLRSKKYDEFKDVELRPGSLIPLEIGTATQLD
ISATFEIDEKKLESTLEADVLFNCTTSEGSVGRGVLGPFGIVVLADANRSEQLPVYFYIAKDTDGTSRTYFCADE
SRSSKDKDVGKWVYGSSVPVLEGENYNMRLLVDHSIVEGFAQGGRTVVTSRVYPTMAIYGAAKIFLFNNATGISV
KASLKIWKMAEAQLDPFPLSGWSS (SEQ ID NO: 38)
MGSHGKPPLPYAYKPLPSDADGERTGCTRWRVCATALTASAMVVVVVGATLLAGFRVDQAVDEEAAGGFPWSNEM
LQWQRSGYHFQTAKNYMSDPNGLMYYRGWYHMFFQYNPVGTDWDDGMEWGHAVSRNLVQWRTLPIAMVADQWYDI
LGVLSGSMTVLPNGTVIMIYTGATNASAVEVQCIATPADPNDPLLRRWTKHPANPVIWSPPGVGTKDFRDSMTAW
YDESDDTWRTLLGSKDDNNGHHDGIAMMYKTKDFLNYELIPGILHRVERTGEWECIDFYPVGHRTSDNSSEMLHV
LKASMDDERHDYYSLGTYDSAANRWTPIDPELDLGIGLRYDWGKFYASTSFYDPAKKRRVLMGYVGEVDSKRADV
VKGWASIQSVPRTIALDEKTRTNLLLWPVEEIETLRLNATQLSDVTLNTGSVIHIPLRQGTQLDIEATFHLDASA
VAALNEADVGYNCSSSGGAVNRGALGPFGLLVLAAGDRRGEQTAVYFYVSRGLDGGLHTSFCQDELRSSRAKDVT
KRVIGSTVPVLDGEAFSMRVLVDHSIVQGFAMGGRTTMTSRVYPMEAYQEAKVYLFNNATGASVTAERLVVHDMD
SAHNQLSNMDDYSYVQ (SEQ ID NO: 15; secretion signal is underlined)
MRFPSIFTAVLFAASSALAAPVNTTTEDETAQIPAEAVIGYSDLEGDFDVAVLPFSNSTNNGLLFINTTIASIAA
KEEGVSLEKREAEADEEAAGGFPWSNEMLQWQRSGYHFQTAKNYMSDPNGLMYYRGWYHMFFQYNPVGTDWDDGM
EWGHAVSRNLVQWRTLPIAMVADQWYDILGVLSGSMTVLPNGTVIMIYTGATNASAVEVQCIATPADPNDPLLRR
WTKHPANPVIWSPPGVGTKDFRDSMTAWYDESDDTWRTLLGSKDDNNGHHDGIAMMYKTKDFLNYELIPGILHRV
ERTGEWECIDFYPVGHRTSDNSSEMLHVLKASMDDERHDYYSLGTYDSAANRWTPIDPELDLGIGLRYDWGKFYA
STSFYDPAKKRRVLMGYVGEVDSKRADVVKGWASIQSVPRTIALDEKTRTNLLLWPVEEIETLRLNATQLSDVTL
NTGSVIHIPLRQGTQLDIEATFHLDASAVAALNEADVGYNCSSSGGAVNRGALGPFGLLVLAAGDRRGEQTAVYF
YVSRGLDGGLHTSFCQDELRSSRAKDVTKRVIGSTVPVLDGEAFSMRVLVDHSIVQGFAMGGRTTMTSRVYPMEA
YQEAKVYLFNNATGASVTAERLVVHDMDSAHNQLSNMDDYSYVQ (SEQ ID NO: 39; secretion
signal is underlined)
DEEAAGGFPWSNEMLQWQRSGYHFQTAKNYMSDPNGLMYYRGWYHMFFQYNPVGTDWDDGMEWGHAVSRNLVQWR
TLPIAMVADQWYDILGVLSGSMTVLPNGTVIMIYTGATNASAVEVQCIATPADPNDPLLRRWTKHPANPVIWSPP
GVGTKDFRDSMTAWYDESDDTWRTLLGSKDDNNGHHDGIAMMYKTKDFLNYELIPGILHRVERTGEWECIDFYPV
GHRTSDNSSEMLHVLKASMDDERHDYYSLGTYDSAANRWTPIDPELDLGIGLRYDWGKFYASTSFYDPAKKRRVL
MGYVGEVDSKRADVVKGWASIQSVPRTIALDEKTRTNLLLWPVEEIETLRLNATQLSDVTLNTGSVIHIPLRQGT
QLDIEATFHLDASAVAALNEADVGYNCSSSGGAVNRGALGPFGLLVLAAGDRRGEQTAVYFYVSRGLDGGLHTSF
CQDELRSSRAKDVTKRVIGSTVPVLDGEAFSMRVLVDHSIVQGFAMGGRTTMTSRVYPMEAYQEAKVYLFNNATG
ASVTAERLVVHDMDSAHNQLSNMDDYSYVQ (SEQ ID NO: 40)
MGSHGKPPLPYAYKPLPSDADGERTGCTRWRVCAVALTASAMVVVVVGATLLAGFRVDQAVDEEAAGGFPWSNEM
LQWQRSGYHFQTAKNYMSDPNGLMYYRGWNHMFFQYNPVGTDWDDGMEWGHAVSRNLVQWRTLPIAMVADQWYDI
LGVLSGSMTVLPNGTVIMIYTGATNASAVEVQCIATPADPTDPLLRRWTKHPANPVIWSPPGVGTKDFRDPMTAW
YDESDDTWRTLLGSKDDNNGHHDGIAMMYKTKDFLNYELIPGILHRVERTGEWECIDFYPVGRRTSDNSSEMLHV
LKASMDDERHDYYSLGTYDSAANRWTPIDPELDLGIGLRYDWGKFYASTSFYDPAKKRRVLMGYVGEVDSKRADV
VKGWASIQSVPRTIALDEKTRTNLLLWPVEEIETLRLNATELSDVTLNTGSVIHIPLRQGTQLDIEATFHLDASA
VAAFNEADVGYNCSSSGGAVNRGALGPFGLLVLAAGDRRGEQTAVYFYVSRGLDGGLHTSFCQDELRSSRAKDVT
KRVIGSTVPVLDGEAFSMRVLVDHSIVQGFAMGGRTTMTSRVYPMEAYQEAKVYLFNNATGASVTAERLVVHEMD
SAHNQLSNMDDHSYVQ (SEQ ID NO: 16; secretion signal is underlined)
MRFPSIFTAVLFAASSALAAPVNTTTEDETAQIPAEAVIGYSDLEGDFDVAVLPFSNSTNNGLLFINTTIASIAA
KEEGVSLEKREAEADEEAAGGFPWSNEMLQWQRSGYHFQTAKNYMSDPNGLMYYRGWNHMFFQYNPVGTDWDDGM
EWGHAVSRNLVQWRTLPIAMVADQWYDILGVLSGSMTVLPNGTVIMIYTGATNASAVEVQCIATPADPTDPLLRR
WTKHPANPVIWSPPGVGTKDFRDPMTAWYDESDDTWRTLLGSKDDNNGHHDGIAMMYKTKDFLNYELIPGILHRV
ERTGEWECIDFYPVGRRTSDNSSEMLHVLKASMDDERHDYYSLGTYDSAANRWTPIDPELDLGIGLRYDWGKFYA
STSFYDPAKKRRVLMGYVGEVDSKRADVVKGWASIQSVPRTIALDEKTRTNLLLWPVEEIETLRLNATELSDVTL
NTGSVIHIPLRQGTQLDIEATFHLDASAVAAFNEADVGYNCSSSGGAVNRGALGPFGLLVLAAGDRRGEQTAVYF
YVSRGLDGGLHTSFCQDELRSSRAKDVTKRVIGSTVPVLDGEAFSMRVLVDHSIVQGFAMGGRTTMTSRVYPMEA
YQEAKVYLFNNATGASVTAERLVVHEMDSAHNQLSNMDDHSYVQ (SEQ ID NO: 41; secretion
signal is underlined)
DEEAAGGFPWSNEMLQWQRSGYHFQTAKNYMSDPNGLMYYRGWNHMFFQYNPVGTDWDDGMEWGHAVSRNLVQWR
TLPIAMVADQWYDILGVLSGSMTVLPNGTVIMIYTGATNASAVEVQCIATPADPTDPLLRRWTKHPANPVIWSPP
GVGTKDFRDPMTAWYDESDDTWRTLLGSKDDNNGHHDGIAMMYKTKDFLNYELIPGILHRVERTGEWECIDFYPV
GRRTSDNSSEMLHVLKASMDDERHDYYSLGTYDSAANRWTPIDPELDLGIGLRYDWGKFYASTSFYDPAKKRRVL
MGYVGEVDSKRADVVKGWASIQSVPRTIALDEKTRTNLLLWPVEEIETLRLNATELSDVTLNTGSVIHIPLRQGT
QLDIEATFHLDASAVAAFNEADVGYNCSSSGGAVNRGALGPFGLLVLAAGDRRGEQTAVYFYVSRGLDGGLHTSF
CQDELRSSRAKDVTKRVIGSTVPVLDGEAFSMRVLVDHSIVQGFAMGGRTTMTSRVYPMEAYQEAKVYLFNNATG
ASVTAERLVVHEMDSAHNQLSNMDDHSYVQ (SEQ ID NO: 42)
MESSRGILIPGTPPLPYAYEPLPSSLTDANGQEDRRITGGVRWRAWAAVLAVGALVVAAAVFGASRVDRDAVASS
VPATAEHGVLEKASGPYSASGGFPWSNAMLQWQRTGYHFQPEKNYQNDPNGPVYYKGWYHFFYQHNPGGTGWGNI
SWGHAVSRDMVHWRHLPLAMVPEHWYDIEGVLTGSITVLPDGRVILLYTGNTETFAQVTCLAEAADPSDPLLREW
AKHPANPVVYPPPGIGMKDYRDPTTAWFDNSDNTWRIIIGSKNDTDHSGIVFTYKTKDFVSYELIPGYLYRGPAG
TGMYECIDLFAVGGGRAASDMYNSTAEDVLYVLKESSDDDRRDYYALGRFDAAANTWTPIDTERELGVALRYDYG
RYDTSKSFYDPVKQRRIVWGYVVETDSWSADAAKGWANLQSIPRTVELDEKTRTNLVQWPVGELNTLRINTTDLS
DITVGAGSVDSLPLHQTSQLDIEASFRINASTIEALNEVDVGYNCTMTSGAATRGALGPFGILVLANVALTEQTA
VYFYVSKGLDGGLRTHFCHDELRSTHATDVAKEVVGSTVPVLDGEDFSVRVLVDHSIVQSFVMGGRMTATSRAYP
TEAIYAAAGVYLFNNATGASITAEKLVVHDMDSSYNRIFTDEDLLVLD (SEQ ID NO: 17; secretion
signal is underlined)
MRFPSIFTAVLFAASSALAAPVNTTTEDETAQIPAEAVIGYSDLEGDFDVAVLPFSNSTNNGLLFINTTIASIAA
KEEGVSLEKREAEASGPYSASGGFPWSNAMLQWQRTGYHFQPEKNYQNDPNGPVYYKGWYHFFYQHNPGGTGWGN
ISWGHAVSRDMVHWRHLPLAMVPEHWYDIEGVLTGSITVLPDGRVILLYTGNTETFAQVTCLAEAADPSDPLLRE
WAKHPANPVVYPPPGIGMKDYRDPTTAWFDNSDNTWRIIIGSKNDTDHSGIVFTYKTKDFVSYELIPGYLYRGPA
GTGMYECIDLFAVGGGRAASDMYNSTAEDVLYVLKESSDDDRRDYYALGRFDAAANTWTPIDTERELGVALRYDY
GRYDTSKSFYDPVKQRRIVWGYVVETDSWSADAAKGWANLQSIPRTVELDEKTRTNLVQWPVGELNTLRINTTDL
SDITVGAGSVDSLPLHQTSQLDIEASFRINASTIEALNEVDVGYNCTMTSGAATRGALGPFGILVLANVALTEQT
AVYFYVSKGLDGGLRTHFCHDELRSTHATDVAKEWGSTVPVLDGEDFSVRVLVDHSIVQSFVMGGRMTATSRAY
PTEAIYAAAGVYLFNNATGASITAEKLVVHDMDSSYNRIFTDEDLLVLD (SEQ ID NO: 43; secretion
signal is underlined)
SGPYSASGGFPWSNAMLQWQRTGYHFQPEKNYQNDPNGPVYYKGWYHFFYQHNPGGTGWGNISWGHAVSRDMVHW
RHLPLAMVPEHWYDIEGVLTGSITVLPDGRVILLYTGNTETFAQVTCLAEAADPSDPLLREWAKHPANPVVYPPP
GIGMKDYRDPTTAWFDNSDNTWRI1IGSKNDTDHSGIVFTYKTKDFVSYELIPGYLYRGPAGTGMYECIDLFAVG
GGRAASDMYNSTAEDVLYVLKESSDDDRRDYYALGRFDAAANTWTPIDTERELGVALRYDYGRYDTSKSFYDPVK
QRRIVWGYVVETDSWSADAAKGWANLQSIPRTVELDEKTRTNLVQWPVGELNTLRINTTDLSDITVGAGSVDSLP
LHQTSQLDIEASFRINASTIEALNEVDVGYNCTMTSGAATRGALGPFGILVLANVALTEQTAVYFYVSKGLDGGL
RTHFCHDELRSTHATDVAKEWGSTVPVLDGEDFSVRVLVDHSIVQSFVMGGRMTATSRAYPTEAIYAAAGVYLF
NNATGASITAEKLVVHDMDSSYNRIFTDEDLLVLD (SEQ ID NO: 44)
MANAFPWSNAMLQWQRTGFHFQPDKYYQNDPNGPVYYGGWYHFFYQYNPSGSVWEPQIVWGHAVSKDLIHWRHLP
PALVPDQWYDIKGVLTGSITVLPDGKVILLYTGNTETFAQVTCLAEPADPSDPLLREWVKHPANPVVFPPPGIGM
KDFRDPTTAWYDESDGTWRTIIGSKNDSDHSGIVFSYKTKDFISYELMPGYMYRGPKGTGEYECIDLYAVGGGRK
ASDMYNSTAEDVLYVLKESSDDDRHDWYSLGRFDAAANKWTPIDTELELGVGLRYDWGKYYASKSFYDPVKKRRV
VWAYVGETDSERADITKGWANLQSIPRTVELDEKTRTNLIQWPVEELNTLRINTTDLSGITVGAGSVAFLPLHQT
AQLDIEATFRIDASAIEALNEADVSYNCTTSRGAATRGALGPFGLLVLANHALTEQTGVYFYVSKGLDGGLRTHF
CHDELRSSHASDVVKRVVGSTVPVLDGEDFSVRVLVDHSIVQSFAMGGRLTATSRAYPTEAIYAAAGVYMFNNAT
GTSVTAEKLVVHDMDSSYNHIYTDGDLVVVD (SEQ ID NO: 18)
MRFPSIFTAVLFAASSALAAPVNTTTEDETAQIPAEAVIGYSDLEGDFDVAVLPFSNSTNNGLLFINTTIASIAA
KEEGVSLEKREAEAANAFPWSNAMLQWQRTGFHFQPDKYYQNDPNGPVYYGGWYHFFYQYNPSGSVWEPQIVWGH
AVSKDLIHWRHLPPALVPDQWYDIKGVLTGSITVLPDGKVILLYTGNTETFAQVTCLAEPADPSDPLLREWVKHP
ANPVVFPPPGIGMKDFRDPTTAWYDESDGTWRTIIGSKNDSDHSGIVFSYKTKDFISYELMPGYMYRGPKGTGEY
ECIDLYAVGGGRKASDMYNSTAEDVLYVLKESSDDDRHDWYSLGRFDAAANKWTPIDTELELGVGLRYDWGKYYA
SKSFYDPVKKRRVVWAYVGETDSERADITKGWANLQSIPRTVELDEKTRTNLIQWPVEELNTLRINTTDLSGITV
GAGSVAFLPLHQTAQLDIEATFRIDASAIEALNEADVSYNCTTSRGAATRGALGPFGLLVLANHALTEQTGVYFY
VSKGLDGGLRTHFCHDELRSSHASDVVKRVVGSTVPVLDGEDFSVRVLVDHSIVQSFAMGGRLTATSRAYPTEAI
YAAAGVYMFNNATGTSVTAEKLVVHDMDSSYNHIYTDGDLVVVD (SEQ ID NO: 45; secretion
signal is underlined)
ANAFPWSNAMLQWQRTGFHFQPDKYYQNDPNGPVYYGGWYHFFYQYNPSGSVWEPQIVWGHAVSKDLIHWRHLPP
ALVPDQWYDIKGVLTGSITVLPDGKVILLYTGNTETFAQVTCLAEPADPSDPLLREWVKHPANPVVFPPPGIGMK
DFRDPTTAWYDESDGTWRTIIGSKNDSDHSGIVFSYKTKDFISYELMPGYMYRGPKGTGEYECIDLYAVGGGRKA
SDMYNSTAEDVLYVLKESSDDDRHDWYSLGRFDAAANKWTPIDTELELGVGLRYDWGKYYASKSFYDPVKKRRVV
WAYVGETDSERADITKGWANLQSIPRTVELDEKTRTNLIQWPVEELNTLRINTTDLSGITVGAGSVAFLPLHQTA
QLDIEATFRIDASAIEALNEADVSYNCTTSRGAATRGALGPFGLLVLANHALTEQTGVYFYVSKGLDGGLRTHFC
HDELRSSHASDWKRWGSTVPVLDGEDFSVRVLVDHSIVQSFAMGGRLTATSRAYPTEAIYAAAGVYMFNNATG
TSVTAEKLVVHDMDSSYNHIYTDGDLVVVD (SEQ ID NO: 46)
MESRDIESSPALNAPLLQASPPIKSSKLKVALLATSTSVLLLIAAFFAVKYSVFDSGSGLLKDDPPSDSEDYPWT
NEMLKWQRTGYHFQPPNHFMADPNAAMYYKGWYHFFYQYNPNGSAWDYSISWGHAVSKDMIHWLHLPVAMVPDHW
YDSKGVWSGYATTLPDGRIIVLYTGGTDQLVQVQNLAEPADPSDPLLIEWKKSNGNPILMPPPGVGPHDFRDPFP
VWYNESDSTWHMLIGSKDDNHYGTVLIYTTKDFETYTLLPDILHKTKDSVGMLECVDLYPVATTGNQIGNGLEMK
GGSGKGIKHVLKASMDDERHDYYAIGTFDLESFSWVPDDDTIDVGVGLRYDYGKFYASKTFYDQEKKRRILWGYV
GEVDSKADDILKGWASVQNIARTILFDAKTRSNLLVWPVEELDALRTSGKEFNGVVVEPGSTYHLDVGTATQLDI
EAEFEINKEAVDAVVEADVTYNCSTSDGAAHRGLLGPFGLLVLANEKMTEKTATYFYVSRNVDGGLQTHFCQDEL
RSSKANDITKRVVGHTVPVLHGETFSLRILVDHSIVESFAQKGRAVATSRVYPTEAIYDSTRVFLFNNATSATVT
AKSVKIWHMNSTHNHPFPGFPAP (SEQ ID NO: 19; secretion signal is underlined)
MRFPSIFTAVLFAASSALAAPVNTTTEDETAQIPAEAVIGYSDLEGDFDVAVLPFSNSTNNGLLFINTTIASIAA
KEEGVSLEKREAEADDPPSDSEDYPWTNEMLKWQRTGYHFQPPNHFMADPNAAMYYKGWYHFFYQYNPNGSAWDY
SISWGHAVSKDMIHWLHLPVAMVPDHWYDSKGVWSGYATTLPDGRIIVLYTGGTDQLVQVQNLAEPADPSDPLLI
EWKKSNGNPILMPPPGVGPHDFRDPFPVWYNESDSTWHMLIGSKDDNHYGTVLIYTTKDFETYTLLPDILHKTKD
SVGMLECVDLYPVATTGNQIGNGLEMKGGSGKGIKHVLKASMDDERHDYYAIGTFDLESFSWVPDDDTIDVGVGL
RYDYGKFYASKTFYDQEKKRRILWGYVGEVDSKADDILKGWASVQNIARTILFDAKTRSNLLVWPVEELDALRTS
GKEFNGVVVEPGSTYHLDVGTATQLDIEAEFEINKEAVDAVVEADVTYNCSTSDGAAHRGLLGPFGLLVLANEKM
TEKTATYFYVSRNVDGGLQTHFCQDELRSSKANDITKRVVGHTVPVLHGETFSLRILVDHSIVESFAQKGRAVAT
SRVYPTEAIYDSTRVFLFNNATSATVTAKSVKIWHMNSTHNHPFPGFPAP (SEQ ID NO: 47;
secretion signal is underlined)
DDPPSDSEDYPWTNEMLKWQRTGYHFQPPNHFMADPNAAMYYKGWYHFFYQYNPNGSAWDYSISWGHAVSKDMIH
WLHLPVAMVPDHWYDSKGVWSGYATTLPDGRIIVLYTGGTDQLVQVQNLAEPADPSDPLLIEWKKSNGNPILMPP
PGVGPHDFRDPFPVWYNESDSTWHMLIGSKDDNHYGTVLIYTTKDFETYTLLPDILHKTKDSVGMLECVDLYPVA
TTGNQIGNGLEMKGGSGKGIKHVLKASMDDERHDYYAIGTFDLESFSWVPDDDTIDVGVGLRYDYGKFYASKTFY
DQEKKRRILWGYVGEVDSKADDILKGWASVQNIARTILFDAKTRSNLLVWPVEELDALRTSGKEFNGVVVEPGST
YHLDVGTATQLDIEAEFEINKEAVDAVVEADVTYNCSTSDGAAHRGLLGPFGLLVLANEKMTEKTATYFYVSRNV
DGGLQTHFCQDELRSSKANDITKRVVGHTVPVLHGETFSLRILVDHSIVESFAQKGRAVATSRVYPTEAIYDSTR
VFLFNNATSATVTAKSVKIWHMNSTHNHPFPGFPAP (SEQ ID NO: 48)
MASSTKDVEAPPTLDAPLLGSAAPRSRLRVAAVSLSVMAFLLVAIAAAVLYYNPGGVASNLMRLRENDYPWTNDM
LRWQRTGFHFQPEKNFQADPNAAMFYKGWYHFFYQYNPTGVAWDYTISWGHAVSKDLLHWNYLPMALRPDHWYDR
KGVWSGYSTLLPDGRIVVLYTGGTKELVQVQNLAVPVNLSDPLLLEWKKSHVNPILVPPPGIEDHDFRDPFPVWY
NESDSRWHVVIGSKDPEHYGIVLIYTTKDFVNFTLLPNILHSTKQPVGMLECVDLFPVATTDSRANQALDMTTMR
PGPGLKYVLKASMDDERHDYYALGSFDLDSFTFTPDDETIDVGVGLRYDWGKFYASKTFYDQEKHRRVLWGYVGE
VDSKRDDALKGWASLQNIPRTILFDTKTKSNLILWPVEEVESLRTINKNFNSIPLYPGSTYQLDVGEATQLDIVA
EFEVDEKAIEATAEADVTYNCSTSGGAANRGVLGPFGLLVLANQELSEQTATYFYVSRGIDGNLRTHFCQDELRS
SKAGAITKRVVGSTVPVLHGETWALRILVDHSIVESFAQRGRAVATSRVYPTEAIYSSARVFLFNNATDAIVTAK
TVNVWHMNSTYNHVFPGLVAP (SEQ ID NO: 20; secretion signal is underlined)
MRFPSIFTAVLFAASSALAAPVNTTTEDETAQIPAEAVIGYSDLEGDFDVAVLPFSNSTNNGLLFINTTIASIAA
KEEGVSLEKREAEANLMRLRENDYPWTNDMLRWQRTGFHFQPEKNFQADPNAAMFYKGWYHFFYQYNPTGVAWDY
TISWGHAVSKDLLHWNYLPMALRPDHWYDRKGVWSGYSTLLPDGRIVVLYTGGTKELVQVQNLAVPVNLSDPLLL
EWKKSHVNPILVPPPGIEDHDFRDPFPVWYNESDSRWHVVIGSKDPEHYGIVLIYTTKDFVNFTLLPNILHSTKQ
PVGMLECVDLFPVATTDSRANQALDMTTMRPGPGLKYVLKASMDDERHDYYALGSFDLDSFTFTPDDETIDVGVG
LRYDWGKFYASKTFYDQEKHRRVLWGYVGEVDSKRDDALKGWASLQNIPRTILFDTKTKSNLILWPVEEVESLRT
INKNFNSIPLYPGSTYQLDVGEATQLDIVAEFEVDEKAIEATAEADVTYNCSTSGGAANRGVLGPFGLLVLANQE
LSEQTATYFYVSRGIDGNLRTHFCQDELRSSKAGAITKRVVGSTVPVLHGETWALRILVDHSIVESFAQRGRAVA
TSRVYPTEAIYSSARVFLFNNATDAIVTAKTVNVWHMNSTYNHVFPGLVAP (SEQ ID NO: 49)
NLMRLRENDYPWTNDMLRWQRTGFHFQPEKNFQADPNAAMFYKGWYHFFYQYNPTGVAWDYTISWGHAVSKDLLH
WNYLPMALRPDHWYDRKGVWSGYSTLLPDGRIVVLYTGGTKELVQVQNLAVPVNLSDPLLLEWKKSHVNPILVPP
PGIEDHDFRDPFPVWYNESDSRWHVVIGSKDPEHYGIVLIYTTKDFVNFTLLPNILHSTKQPVGMLECVDLFPVA
TTDSRANQALDMTTMRPGPGLKYVLKASMDDERHDYYALGSFDLDSFTFTPDDETIDVGVGLRYDWGKFYASKTF
YDQEKHRRVLWGYVGEVDSKRDDALKGWASLQNIPRTILFDTKTKSNLILWPVEEVESLRTINKNFNSIPLYPGS
TYQLDVGEATQLDIVAEFEVDEKAIEATAEADVTYNCSTSGGAANRGVLGPFGLLVLANQELSEQTATYFYVSRG
IDGNLRTHFCQDELRSSKAGAITKRVVGSTVPVLHGETWALRILVDHSIVESFAQRGRAVATSRVYPTEAIYSSA
RVFLFNNATDAIVTAKTVNVWHMNSTYNHVFPGLVAP (SEQ ID NO: 50)
MESRDIESSPALNAPLLQTSPPIKSSKLKVALLATSTSVLLLIAAFFAVKYSVFDSGSGLLKDDPPSDSEDYPWT
NEMLKWQRTGYHFQPPNHFMADPNAAMYYKGWYHFFYQYNPNGSAWDYSISWGHAVSKDMIHWLHLPVAMVPDHW
YDSKGVWSGYATTLPDGRIIVLYTGGTDQLVQVQNLAEPADPSDPLLIEWKKSNGNPILMPPPGVGPHDFRDPFP
VWYNESDSTWHMLIGSKDDNHYGTVLIYTTKDFETYTLLPDILHKTKDSVGMLECVDLYPVATTGNQIGNGLEMK
GGSGKGIKHVLKASMDDERHDYYAIGTFDLESFSWVPDDDTIDVGVGLRYDYGKFYASKTFYDQEKKRRILWGYV
GEVDSKADDILKGWASVQNIARTILFDAKTRSNLLVWPVEELDALRTSGKEFNGVVVEPGSTYHLDVGTATQLDI
EAEFEINKEAVDAVVEADVTYNCSTSDGAAHRGLLGPFGLLVLANEKMTEKTATYFYVSRNADGGLQTHFCQDEL
RSSKANDITKRVVGHTVPVLHGETFSLRILVDHSIVESFAQKGRAVATSRVYPTEAIYDSTRVFLFNNATSATVT
AKSVKIWHMNSTHNHPFPGFPAP (SEQ ID NO: 21; secretion signal is underlined)
MRFPSIFTAVLFAASSALAAPVNTTTEDETAQIPAEAVIGYSDLEGDFDVAVLPFSNSTNNGLLFINTTIASIAA
KEEGVSLEKREAEADDPPSDSEDYPWTNEMLKWQRTGYHFQPPNHFMADPNAAMYYKGWYHFFYQYNPNGSAWDY
SISWGHAVSKDMIHWLHLPVAMVPDHWYDSKGVWSGYATTLPDGRIIVLYTGGTDQLVQVQNLAEPADPSDPLLI
EWKKSNGNPILMPPPGVGPHDFRDPFPVWYNESDSTWHMLIGSKDDNHYGTVLIYTTKDFETYTLLPDILHKTKD
SVGMLECVDLYPVATTGNQIGNGLEMKGGSGKGIKHVLKASMDDERHDYYAIGTFDLESFSWVPDDDTIDVGVGL
RYDYGKFYASKTFYDQEKKRRILWGYVGEVDSKADDILKGWASVQNIARTILFDAKTRSNLLVWPVEELDALRTS
GKEFNGVVVEPGSTYHLDVGTATQLDIEAEFEINKEAVDAVVEADVTYNCSTSDGAAHRGLLGPFGLLVLANEKM
TEKTATYFYVSRNADGGLQTHFCQDELRSSKANDITKRVVGHTVPVLHGETFSLRILVDHSIVESFAQKGRAVAT
SRVYPTEAIYDSTRVFLFNNATSATVTAKSVKIWHMNSTHNHPFPGFPAP (SEQ ID NO: 51;
secretion signal is underlined)
DDPPSDSEDYPWTNEMLKWQRTGYHFQPPNHFMADPNAAMYYKGWYHFFYQYNPNGSAWDYSISWGHAVSKDMIH
WLHLPVAMVPDHWYDSKGVWSGYATTLPDGRIIVLYTGGTDQLVQVQNLAEPADPSDPLLIEWKKSNGNPILMPP
PGVGPHDFRDPFPVWYNESDSTWHMLIGSKDDNHYGTVLIYTTKDFETYTLLPDILHKTKDSVGMLECVDLYPVA
TTGNQIGNGLEMKGGSGKGIKHVLKASMDDERHDYYAIGTFDLESFSWVPDDDTIDVGVGLRYDYGKFYASKTFY
DQEKKRRILWGYVGEVDSKADDILKGWASVQNIARTILFDAKTRSNLLVWPVEELDALRTSGKEFNGVVVEPGST
YHLDVGTATQLDIEAEFEINKEAVDAVVEADVTYNCSTSDGAAHRGLLGPFGLLVLANEKMTEKTATYFYVSRNA
DGGLQTHFCQDELRSSKANDITKRVVGHTVPVLHGETFSLRILVDHSIVESFAQKGRAVATSRVYPTEAIYDSTR
VFLFNNATSATVTAKSVKIWHMNSTHNHPFPGFPAP (SEQ ID NO: 52)
GARVGLGGIYDDADAFAWNNSMLQWQRAGFHFQTEKNFMSDPNGPVYYRGYYHLFYQYNMKGVVWDDGIVWGHVV
SRDLVHWRHLPIAMVPDHWYDSMGVLSGSITVLQNGSLVMIYTGVFSKTTDRSGMMEVQCLAVPADPNDPLLRSW
TKHPANPVLVHPPGIKDMDFRDPTTAWFDESDSTYRTVIGTKDDHHGSHAGFAMVYKTKDFLSFQRIPGILHSVE
HTGMWECMDFYPVGGGDNSSSEVLYVIKASMDDERHDYYALGMYDAAANTWTPLDQELDLGIGLRYDWGKLYAST
TFYDPAKRRRVMLGYVGETDSRRSDEAKGWASIQSIPRTVALDEKTRTNLLLWPVEEIETLRLNATEFNDINIDT
GSVFHLPIRQGNQLDIEASFRLDASAVAAINEADVGYNCSSSGGAATRGALGPFGLLVLAAEGIGEQTAVYFYVS
RGLDGGLRTSFCNDELRSSWARDVTKRVVGSTVPVLNGETLSMRVLVDHSIVQSFAMGGRVTATSRVYPTEAIYA
AAGVYLFNNATNASVTAERIIVHEMDSIDNNQIFLIDDL (SEQ ID NO: 63)
atgagatttccttcaatttttactgctgttttattcgcagcatcctccgcattagctgctccagtcaacactaca
acagaagatgaaacggcacaaattccggctgaagctgtcatcggttactcagatttagaaggggatttcgatgtt
gctgttttgccattttccaacagcacaaataacgggttattgtttataaatactactattgccagcattgctgct
aaagaagaaggggtatctctcgagaaaagagaggctgaagctgtacccggtaaattagaatcgaatgccgatgtc
gagtggcaacgttctgcataccattttcagccagacaagaacttcatatccgatcctgacggcccaatgtatcac
atgggatggtaccacctattctaccaatataacccggaatcagctatttgggggaatatcacttggggtcatagt
gtgtctagggacatgattaactggtttcacttgccattcgctatggttccagatcattggtacgacatcgaaggt
gttatgaccggtagcgctacggttcttcctaacggtcaaatcattatgttgtatactggtaatgcgtacgatttg
tctcaattgcaatgcttagcttatgccgtcaactcctcagatccactactcttggaatggaagaagtacgaaggt
aatccaatattgttcccaccacccggtgtcggttacaaagactttagagatccttccaccttatggatgggccca
gacggcgaatggagaatggttatgggtagtaagcacaacgagacaatcggatgtgctttggtctatcgaactacc
aatttcactcactttgaacttaacgaagaagttttacatgctgtaccacacacaggaatgtgggaatgtgtggat
ctctacccggtcagcacgacccatactaacgggttggaaatgaaggacaatggtccaaacgttaaatatatttta
aagcaatctggtgatgaggatagacacgactggtacgccattggtacattcgatccagaaaaggacaaatggtac
cctgatgacccagagaatgacgttggtatcggtttgagatacgactatgggaagttctatgccagtaagactttt
tacgatcaacataaaaagcggagagtattgtggggttacgttggtgaaactgatccaccaaagtcggatctattg
aaaggttgggctaacattctcaacatccctagatcagtcgttttggatacccagacagagactaatttgattcaa
tggccaatcgaagaagttgaaaaacttagatccaagaagtacgacgaatttaaggacgtcgaactgcgtcctggt
tctttgattccattggaaatcggtaccgctacccaattggatatatctgcaactttcgaaattgatgaaaagaaa
ctggagtctactttagaagctgacgttttattcaactgtacaacttcagaaggttccgtcggtagaggtgttcta
ggccctttcggtatcgttgtcttggctgatgctaacagatccgaacaattgccagtttacttctacattgcaaag
gacaccgatggtacttctcgcacctatttctgtgctgacgaatctcgttcttcgaaggataaggatgtgggtaag
tgggtttacggatcttccgtaccagtcctggagggtgaaaactataatatgagattgctcgtcgatcattcgatt
gtagaaggttttgcccaagggggtagaaccgttgtcacctctcgcgtttatccaacgatggcaatctacggtgcc
gctaagatatttttgttcaacaatgctaccggtatttcagtgaaggctagtttaaaaatctggaagatggctgag
gcccaattggaccccttcccactttccggttggagcagttaa (SEQ ID NO: 22)
atgagatttccttcaatttttactgctgttttattcgcagcatcctccgcattagctgctccagtcaacactaca
acagaagatgaaacggcacaaattccggctgaagctgtcatcggttactcagatttagaaggggatttcgatgtt
gctgttttgccattttccaacagcacaaataacgggttattgtttataaatactactattgccagcattgctgct
aaagaagaaggggtatctctcgagaaaagagaggctgaagctgacgaagaggctgccggtggatttccctggtca
aacgaaatgttacaatggcagagatccggttaccacttccaaacagcaaaaaattatatgtctgatcctaacggc
ctaatgtactataggggttggtaccatatgttcttccaatacaacccagtcgggactgattgggacgacggtatg
gaatggggtcacgctgtgtcgcgtaatttggtacaatggagaacgttgccaatagctatggttgccgatcaatgg
tatgatattctgggtgttctttctggttctatgaccgtcttgccaaacggtactgttatcatgatctacaccggt
gctactaatgcgagcgctgtcgaagttcaatgtattgcaaccccagccgatccgaacgaccctttgttaagaaga
tggactaagcatccagctaaccctgtgatctggagtccaccaggtgtagggacaaaggattttcgagactccatg
accgcttggtacgacgagtcagatgacacttggagaaccttgttgggctccaaggacgataacaatggtcaccat
gatggtattgctatgatgtataaaactaaggatttcctaaattacgaacttatcccaggcatactgcaccgtgtc
gaaaggacaggtgaatgggaatgcatcgacttttacccggttggtcatagaacgtctgataactctagcgaaatg
ttgcacgttttgaaagcctctatggatgacgaacggcacgattattactccttaggtacttacgatagtgctgcc
aacagatggaccccaattgaccccgaactagacttgggtattggattgagatatgattggggtaagttttacgct
agcacttcattctacgatccagcaaagaaacgtcgagtcttaatgggatatgttggtgaggttgactccaagaga
gctgacgtcgtgaagggttgggcttctatccaatctgttccaagaacaattgcattggacgaaaagactagaacc
aacctgctgttatggcccgttgaggaaatcgaaacattgagactaaatgctacccaactctcggatgtcaccttg
aatactggttctgtcattcatattcctttgagacaaggtacccagttggatatagaagctacattccaccttgat
gcctccgctgttgccgctttaaacgaagcggacgtcggttacaactgttcctcttctggtggtgctgtgaataga
ggagctttgggtccattcggtttgttagttctcgcggctggagacagacgtggtgagcaaactgctgtttacttt
tatgttagtagaggtttggacggcggtttgcatacctccttctgtcaagatgaactcagaagttcccgcgcgaag
gatgttactaaaagagtcatcggttcgactgtcccggttcttgacggcgaagcattctctatgagggttttagtt
gatcattcgattgtccaaggttttgcaatgggtggtagaactacgatgacatctcgggtctatccaatggaagct
taccaggaggccaaggtttacctctttaacaacgctaccggagcatccgttaccgctgaaagacttgtagttcac
gatatggactcagcccataatcaattgtctaacatggacgactactcatatgtacagtaa (SEQ ID NO:
53)
atgagatttccttcaatttttactgctgttttattcgcagcatcctccgcattagctgctccagtcaacactaca
acagaagatgaaacggcacaaattccggctgaagctgtcatcggttactcagatttagaaggggatttcgatgtt
gctgttttgccattttccaacagcacaaataacgggttattgtttataaatactactattgccagcattgctgct
aaagaagaaggggtatctctcgagaaaagagaggctgaagctgacgaagaggctgccggtggatttccctggtca
aacgaaatgttacaatggcagagatccggttaccacttccaaacagcaaaaaattatatgtctgatcctaacggc
ctaatgtactataggggttggaaccatatgttcttccaatacaatccagtcgggactgattgggacgacggtatg
gaatggggtcacgctgtgtcgcgtaacttggtacaatggagaacgttgccaatagctatggttgccgatcaatgg
tacgatattctgggtgttctttctggttctatgaccgtcttgccaaatggtactgttatcatgatctataccggt
gctactaacgcgagcgctgtcgaagttcaatgtattgcaaccccagccgatccgacggaccctttgttaagaaga
tggactaagcatccagctaaccctgtgatctggagtccaccaggtgtagggacaaaggattttcgagatccaatg
accgcttggtacgacgaatcagacgatacttggagaacgctattgggctctaaggatgacaataatggtcaccac
gacggtattgctatgatgtacaaaactaaggatttcttgaactacgagctgattcctggtatcctccatagagtt
gaaagaacaggagaatgggaatgcatagacttttatccggtcggtcgtagaacctctgataactcgtccgaaatg
ttgcatgttttaaaggcttccatggatgacgagagacacgactactactctctaggtacttatgatagtgccgcc
aataggtggactccaattgacccagaattggatttgggtattggtttgagatatgactgggggaaattctacgct
tccaccagcttctatgatcccgcaaagaagagaagagttttgatgggttacgtcggtgaagtggactctaaacgc
gctgacgttgttaagggttgggcctctatccaaagtgtcccacgcaccattgctctggacgaaaaaactcgtaca
aaccttttattgtggccagtagaagaaatcgaaaccttaagattgaacgctactgagttgtccgacgttacttta
aacactggttccgtcatccacattccattgagacagggaacccaattggatattgaagcaacctttcatctcgat
gcgagtgctgttgcagctttcaatgaagctgatgtcggttacaattgttcatcttcgggtggtgctgttaataga
ggtgctctagggcctttcggcctcttagtcttggctgccggtgatagaagaggtgaacaaaccgctgtttacttt
tacgtatctcgtggtttggacggcggtctacacacctctttttgtcaggatgagttaagatcctcaagggctaag
gacgttactaagagagtcataggatcaactgtgcccgttttggatggtgaagccttttctatgcgtgtacttgtt
gatcattccatagtccaaggtttcgcaatgggtggtagaacaactatgacgagcagagtttatccaatggaagcg
taccaagaagctaaggtttatcttttcaacaacgcaacaggtgcctctgttacagccgagagattggtcgtacac
gaaatggactccgcccacaaccaattgtcgaacatggacgaccactcgtatgttcaataa (SEQ ID NO:
54)
atgagatttccttcaatttttactgctgttttattcgcagcatcctccgcattagctgctccagtcaacactaca
acagaagatgaaacggcacaaattccggctgaagctgtcatcggttactcagatttagaaggggatttcgatgtt
gctgttttgccattttccaacagcacaaataacgggttattgtttataaatactactattgccagcattgctgct
aaagaagaaggggtatctctcgagaaaagagaggctgaagctagtggcccttattctgcttcgggtggttttcca
tggtctaatgccatgttgcagtggcaacgtacaggataccacttccaacccgaaaaaaactaccaaaacgaccca
aacggtccagtctactataagggttggtatcatttcttttaccaacataatccaggtggtaccgggtggggtaac
atctcatggggtcacgcagtttccagagatatggtacactggaggcatttaccactagctatggttcctgagcat
tggtacgatatagaaggtgttttgactggaagcattactgtccttccagacggtagagtcattttgttatatacc
ggcaatactgaaacgttcgctcaagtgacctgtttggcggaggctgccgacccttccgatccactgttgagagaa
tgggctaagcacccggccaacccagtagtttacccgccaccaggtatcggtatgaaagactacagagatccaact
acagcttggttcgataactcagacaatacctggagaataatcattggttctaagaatgatactgatcactctggt
atcgtttttacttacaagaccaaggacttcgtcagctacgaactgattcctggatacctatatagaggtccagcc
gggacgggtatgtacgaatgcattgatttgttcgctgttggtggtgggcgtgctgcatcagatatgtataactct
accgctgaagatgtcttatacgttttgaaagaatcctccgacgacgacagacgggattactatgccttagggcga
tttgacgctgccgctaatacttggacacccatagatacagaaagagagttgggtgtcgcactcagatatgattac
ggtagatacgatacttctaagtctttctacgacccagttaagcaaaggagaattgtctggggttacgttgtcgaa
accgacagttggtccgctgacgctgcaaaaggttgggctaacctgcaatctatccctagaactgttgaattggat
gaaaagactcgaacaaaccttgtacagtggccagtgggtgagttgaacaccctacgtatcaataccactgatttg
agtgacattaccgttggtgctggctcggtcgattctttacccttgcaccaaacttcccaactagacatcgaagcg
tcatttagaattaatgcctctactatagaagccttgaacgaagttgatgtaggttataactgtactatgacgtct
ggtgctgctactagaggtgctttgggtccattcggaattttagtcttggctaacgtggccttgacagaacagacc
gctgtttatttttatgtttccaagggtttagacggtggtttacgaacccacttctgtcatgacgaattgaggtct
acacacgctaccgacgtcgccaaggaggttgttgggtctactgttccagttctcgatggtgaagattttagcgtc
agagttttggtcgatcactcaatcgtacaatctttcgtcatgggtggcagaatgacagcaacttccagagcttac
ccgactgaagcaatctatgctgccgctggcgtttacctcttcaacaatgctacaggtgcttccattaccgcagaa
aaattggtggtacatgacatggattcctcctacaacagaatctttactgacgaggatttattggtgcttgactaa
(SEQ ID NO: 55)
atgagatttccttcaatttttactgctgttttattcgcagcatcctccgcattagctgctccagtcaacactaca
acagaagatgaaacggcacaaattccggctgaagctgtcatcggttactcagatttagaaggggatttcgatgtt
gctgttttgccattttccaacagcacaaataacgggttattgtttataaatactactattgccagcattgctgct
aaagaagaaggggtatctctcgagaaaagagaggctgaagctgcaaatgcttttccttggtcgaacgctatgttg
cagtggcaacgtactggcttccatttccaaccagacaaatactatcaaaacgatccaaacggtcccgtctactac
ggaggttggtatcactttttctaccaatataatccgtctggtagtgtttgggagccacaaattgtatggggtcac
gccgtttccaaggacctgatccattggcggcacttaccaccagctttggtcccagatcaatggtacgacataaag
ggtgttctaaccgggtcaattacggtccttcctgatggtaaggtgatcttgttatatactggtaatacagaaacc
ttcgctcaagttacttgcttggccgaacccgcagatccaagcgatccattgctcagagaatgggtaaagcatcct
gctaacccagttgtctttccaccacccggtattggtatgaaagacttcagagatccaaccactgcttggtacgac
gaatctgacggcacatggagaaccatcattggatctaaaaacgactccgaccactctggtatcgttttttcctac
aagactaaggatttcattagttatgagttgatgccgggttacatgtacagaggcccaaaggggaccggtgaatac
gaatgtatagatttatacgcggtgggtggtggtaggaaggcttctgatatgtataactccactgcggaagatgtc
ctatatgttttaaaagaatcatctgacgatgatagacatgactggtactcattgggtagatttgacgccgctgct
aataagtggacacctatagatactgagcttgaacttggcgttggtttgcgatatgactggggtaagtactacgcc
agcaagtctttctacgacccagttaaaaaaagacgtgtcgtgtgggcttatgtcggtgaaaccgattccgaaaga
gccgacatcaccaagggttgggcaaatttgcagtctatcccacgcactgttgaattggacgaaaaaactagaacg
aacttaattcaatggccggttgaggaactaaatacactgcgtattaacactacagatttgtcgggaatcaccgta
ggtgctggtagtgtcgctttcttgccattgcaccaaactgcccagctcgacattgaagctacttttagaattgat
gcttctgcgatagaagctctaaacgaagctgatgtttcctacaattgtaccacatcgcgaggagctgctaccaga
ggtgccttaggtccattcggtttgttggtattagccaaccatgccttgaccgaacaaactggtgtttacttttac
gtgtctaagggtttggacggtggtttaagaactcacttctgtcacgatgaactaagatcctctcatgcttcagat
gtcgttaagagagtcgtgggtagtacggttcctgttttggatggggaggactttagcgttcgtgtcttggttgac
cactctattgtccaaagtttcgccatgggtggtaggttgacagctacctccagagcttatccaactgaagcaatc
tacgctgcggcaggcgtatacatgttcaacaacgctacaggtacttccgttacggctgaaaagcttgttgtccac
gatatggattcttcctacaaccacatctataccgacggtgacctggtggtagttgattaa (SEQ ID NO:
56)
atgagatttccttcaatttttactgctgttttattcgcagcatcctccgcattagctgctccagtcaacactaca
acagaagatgaaacggcacaaattccggctgaagctgtcatcggttactcagatttagaaggggatttcgatgtt
gctgttttgccattttccaacagcacaaataacgggttattgtttataaatactactattgccagcattgctgct
aaagaagaaggggtatctctcgagaaaagagaggctgaagctgacgatcctccatctgatagtgaagattaccca
tggaccaatgagatgcttaaatggcaaaggacgggttatcacttccagcccccaaaccattttatggcagaccca
aacgccgctatgtactacaaggggtggtatcacttcttttaccaatataaccctaatggttcagcttgggactac
tccatctcgtggggtcatgctgtatctaaggacatgattcactggctgcatttaccagtcgccatggttccagat
cattggtacgatagcaaaggagtttggtccggctacgctactactttgccagatggtagaataattgtcttgtat
accggtggtacagaccaattggttcaagtgcaaaatttagccgaaccagcggacccttctgatccactattgatc
gaatggaagaagtcaaacggaaacccaattttgatgcctccgccgggtgtaggtccacacgatttcagagatcca
ttcccagtttggtacaacgaatctgactccacatggcacatgttgatcggttctaaagatgacaatcactacggt
accgttctaatttatactactaaggattttgagacatacactttattgccagacatcctacataagaccaaggac
tcggttggtatgttggaatgtgtcgatctttatccagtggctactaccgggaatcaaattggtaacggtttagaa
atgaaaggtggttccggcaagggtatcaagcacgtcctgaaggcttctatggacgatgaacgtcacgattattac
gccataggtacgttcgacttggaatcctttagttgggttccggacgacgataccatagatgtcggcgtcggcttg
cgctatgactacggtaagttctacgcttcaaaaactttctatgatcaggaaaagaagagaagaattttgtgggga
tacgttggtgaagtagactctaaggctgacgacatcttaaaaggttgggcgagcgttcaaaatattgcaagaact
atcctatttgatgcaaaaactagaagtaacttgctcgtctggcccgtcgaggaattggacgctttgcgaacctct
ggtaaggaatttaacggtgtggttgttgaacctggttctacttaccatttagacgtaggtaccgccacccaattg
gatattgaagctgaatttgagatcaataaggaagctgttgacgctgttgtcgaagccgatgttacatacaactgc
tccacatctgatggtgctgctcacagaggtttgttgggaccattcggtcttttggttttagctaatgaaaagatg
acagaaaaaaccgccacttatttctacgtcagtcgtaacgttgatgggggtctacaaactcatttctgtcaagac
gagcttagaagctctaaagctaacgatattaccaaacgtgtcgttggccacactgttccagttctgcatggtgaa
accttctccttgagaattttagtagaccactcgatcgttgaatcgtttgcgcagaagggtagagcagtcgctacg
tctagggtgtatccaactgaagctatctacgattctacaagagttttcctcttcaacaacgccacttcagctacg
gtcactgccaagtccgtaaagatatggcatatgaacagtacccataaccacccttttccaggtttccccgcacca
taa (SEQ ID NO: 57)
atgagatttccttcaatttttactgctgttttattcgcagcatcctccgcattagctgctccagtcaacactaca
acagaagatgaaacggcacaaattccggctgaagctgtcatcggttactcagatttagaaggggatttcgatgtt
gctgttttgccattttccaacagcacaaataacgggttattgtttataaatactactattgccagcattgctgct
aaagaagaaggggtatctctcgagaaaagagaggctgaagctaacttgatgcgtttaagagagaatgattatccc
tggactaacgacatgctaagatggcaacgcacgggatttcacttccagcctgaaaaaaacttccaagccgaccca
aatgcagctatgttttacaagggctggtaccatttcttttatcaatacaacccgaccggtgtggcttgggattac
acaatctcctggggtcacgctgtcagtaaggatttgctgcattggaattatcttccaatggccttgaggcctgac
cactggtacgatagaaaaggtgtttggagcggttactctactttattgccagacggtagaattgttgtcttgtac
accggtggaactaaggaattagttcaagtccaaaacttggctgtcccagtaaacctttctgacccattgctattg
gaatggaagaagtcacacgttaacccaatactcgttccacctccggggatcgaggatcatgatttccgagatcca
ttcccagtgtggtataatgaatctgactcgcggtggcacgttgtaattggttccaaagatccagaacactatggt
attgtcttgatctacactaccaaggacttcgttaactttacgttattaccaaacatattgcattccaccaagcag
ccggttggtatgctggaatgtgtagacttgttcccagttgctacaactgattctcgtgcaaatcaagctttggat
atgactaccatgaggcccggtcctggcctcaaatatgtgttaaaggcgagtatggatgacgaaagacacgattac
tacgccctaggtagctttgacttggactcgttcacttttacaccagatgatgaaaccattgacgtcggtgtcggt
ttgagatacgactggggtaagttctatgcttcaaaaactttctatgaccaagaaaagcatagaagagttttatgg
ggttacgtgggggaagttgattctaagagagatgacgcgttaaaaggctgggcttccttgcaaaacatcccaaga
acaattttgttcgataccaaaactaagtctaatctaatcttgtggccagttgaagaggtcgaatcattgagaact
attaacaagaattttaactctataccactttacccaggttccacttaccaattggatgttggggaagccacccaa
ctggatattgtcgctgaatttgaagtcgatgagaaggctattgaagcaactgctgaagctgacgttacatataac
tgctctaccagcggtggtgccgctaacagaggtgttttgggtcctttcggtctattggttctagccaatcaagaa
ctttccgaacagactgccacttacttctatgtatcgcgtggtatcgacggcaacctgagaacccacttttgtcaa
gacgaattgagatcctccaaagccggtgctatcaccaagagggtcgtaggttctacagttcctgttttgcatggt
gaaacgtgggctttacgtatcctagttgaccactctattgtcgagtcttttgcacaacggggacgcgccgtcgct
accagtagagtatacccaactgaggctatatactcttcggctagagtctttctcttcaataacgcaaccgatgcc
attgttacagctaaaacggtcaacgtttggcatatgaatagcacttacaaccacgtctttcctggtttggttgct
ccataa (SEQ ID NO: 58)
atgagatttccttcaatttttactgctgttttattcgcagcatcctccgcattagctgctccagtcaacactaca
acagaagatgaaacggcacaaattccggctgaagctgtcatcggttactcagatttagaaggggatttcgatgtt
gctgttttgccattttccaacagcacaaataacgggttattgtttataaatactactattgccagcattgctgct
aaagaagaaggggtatctctcgagaaaagagaggctgaagctgacgatcctccatctgatagtgaagattaccca
tggaccaatgagatgcttaaatggcaaaggacgggttatcacttccagcccccaaaccattttatggcagaccca
aacgccgctatgtactacaaggggtggtatcacttcttttaccaatataaccctaatggttcagcttgggactac
tccatctcgtggggtcatgctgtatctaaggacatgattcactggctgcatttaccagtcgccatggttccagat
cattggtacgatagcaaaggagtttggtccggctacgctactactttgccagatggtagaataattgtcttgtat
accggtggtacagaccaattggttcaagtgcaaaatttagccgaaccagcggacccttctgatccactattgatc
gaatggaagaagtcaaacggaaacccaattttgatgcctccgccgggtgtaggtccacacgatttcagagatcca
ttcccagtttggtacaacgaatctgactccacatggcacatgttgatcggttctaaagatgacaatcactacggt
accgttctaatttatactactaaggattttgagacatacactttattgccagacatcctacataagaccaaggac
tcggttggtatgttggaatgtgtcgatctttatccagtggctactaccgggaatcaaattggtaacggtttagaa
atgaaaggtggttccggcaagggtatcaagcacgtcctgaaggcttctatggacgatgaacgtcacgattattac
gccataggtacgttcgacttggaatcctttagttgggttccggacgacgataccatagatgtcggcgtcggcttg
cgctatgactacggtaagttctacgcttcaaaaactttctatgatcaggaaaagaagagaagaattttgtgggga
tacgttggtgaagtagactctaaggctgacgacatcttaaaaggttgggcgagcgttcaaaatattgcaagaact
atcctatttgatgcaaaaactagaagtaacttgctcgtctggcccgtcgaggaattggacgctttgcgaacctct
ggtaaggaatttaacggtgtggttgttgaacctggttctacttaccatttagacgtaggtaccgccacccaattg
gatattgaagctgaatttgagatcaataaggaagctgttgacgctgttgtcgaagccgatgttacatacaactgc
tccacatctgatggtgctgctcacagaggtttgttgggaccattcggtcttttggttttagctaatgaaaagatg
acagaaaaaaccgccacttatttctacgtcagtcgtaacgctgatgggggtctacaaactcatttctgtcaagac
gagcttagaagctctaaagctaacgatattaccaaacgtgtcgttggccacactgttccagttctgcatggtgaa
accttctccttgagaattttagtcgatcactcaattgtcgagtccttcgcgcaaaagggtagggctgttgcaacc
tctcgggtgtatccaactgaagccatctacgattctacgagagtttttctcttcaacaacgctacttcggcaacg
gtaactgctaagtccgtaaagatatggcatatgaacagtacccataaccacccttttccaggtttccccgcgcca
taa (SEQ ID NO: 59)
6-SFT from Phleum pratense:
MAPPQAIANGAPAPLPYAYARLPSSGDEKQDQSKSGGARYCRACVAGVAALLIVAGALAGARVGLGGIYDDADAF
AWNNSMLQWQRAGFHFQTEKNFMSDPNGPVYYRGYYHLFYQYNMKGVVWDDGIVWGHVVSRDLVHWRHLPIAMVP
DHWYDSMGVLSGSITVLQNGSLVMIYTGVFSKTTDRSGMMEVQCLAVPADPNDPLLRSWTKHPANPVLVHPPGIK
DMDFRDPTTAWFDESDSTYRTVIGTKDDHHGSHAGFAMVYKTKDFLSFQRIPGILHSVEHTGMWECMDFYPVGGG
DNSSSEVLYVIKASMDDERHDYYALGMYDAAANTWTPLDQELDLGIGLRYDWGKLYASTTFYDPAKRRRVMLGYV
GETDSRRSDEAKGWASIQSIPRTVALDEKTRTNLLLWPVEEIETLRLNATEFNDINIDTGSVFHLPIRQGNQLDI
EASFRLDASAVAAINEADVGYNCSSSGGAATRGALGPFGLLVLAAEGIGEQTAVYFYVSRGLDGGLRTSFCNDEL
RSSWARDVTKRWGSTVPVLNGETLSMRVLVDHSIVQSFAMGGRVTATSRVYPTEAIYAAAGVYLFNNATNASVT
AERIIVHEMDSIDNNQIFLIDDL (SEQ ID NO: 23)

### EQUIVALENTS

Those skilled in the art will recognize, or be able to ascertain using no more than routine experimentation, many equivalents to the specific embodiments of the invention described in this application. Such equivalents are intended to be encompassed by the following claims.

All references, including patent documents, disclosed in this application are incorporated by reference in their entirety, particularly for the disclosure referenced in this application.

It should be appreciated that sequences disclosed in this application may or may not contain secretion signals. The sequences disclosed in this application encompass versions with or without secretion signals. It should also be understood that protein sequences disclosed in this application may be depicted with or without a start codon (M). The sequences disclosed in this application encompass versions with or without start codons. Accordingly, in some instances amino acid numbering may correspond to protein sequences containing a start codon, while in other instances, amino acid numbering may correspond to protein sequences that do not contain a start codon. It should also be understood that sequences disclosed in this application may be depicted with or without a stop codon. The sequences disclosed in this application encompass versions with or without stop codons. Aspects of the disclosure encompass host cells comprising any of the sequences described in this application and fragments thereof.

1. A host cell that comprises one or more heterologous polynucleotides encoding:
a) a sucrose:sucrose 1-fructosyltransferase (1-SST) enzyme comprising an amino acid sequence that is at least 90% identical to SEQ ID NO: 1 or SEQ ID NO: 24;
b) a fructan:fructan 1-fructosyltransferase (1-FFT) enzyme comprising an amino acid sequence that is at least 90% identical to SEQ ID NO: 7 or SEQ ID NO: 31; and/or
c) a sucrose:fructan-6-fructosyltransferase (6-SFT) enzyme comprising an amino acid sequence that is at least 90% identical to SEQ ID NO: 13 or SEQ ID NO: 38.

2. The host cell of claim 1, wherein the one or more heterologous polynucleotides encode two or more of a), b) and c).

3. The host cell of claim 1, wherein the one or more heterologous polynucleotides encode a), b), and c).

4. The host cell of any one of claims 1-3, wherein the host cell is a plant cell, an algal cell, a yeast cell, a bacterial cell, or an animal cell.

5. The host cell of claim 4, wherein the host cell is a yeast cell.

6. The host cell of claim 5, wherein the yeast cell is a Saccharomyces cell, a Yarrowia cell or a Pichia cell.

7. The host cell of claim 6, wherein the host cell is a Pichia pastoris cell.

8. The host cell of any one of claims 1-7, wherein the 1-SST enzyme comprises the amino acid sequence of SEQ ID NO: 1 or SEQ ID NO: 24.

9. The host cell of any one of claims 1-8, wherein the 1-FFT enzyme comprises the amino acid sequence of SEQ ID NO: 7 or SEQ ID NO: 31.

10. The host cell of any one of claims 1-9, wherein the 6-SFT enzyme comprises the amino acid sequence of SEQ ID NO: 13 or SEQ ID NO: 38.

11. The host cell of any one of claims 1-10, wherein one or more of the 1-SST enzyme, the 1-FFT enzyme, and the 6-SFT enzyme is secreted from the host cell.

12. The host cell of any one of claims 1-11, wherein at least two of the 1-SST, 1-FFT, and 6-SFT enzymes are encoded by the same heterologous polynucleotide.

13. A method comprising culturing the host cell of any one of claims 1-12.

14. The method of claim 13, further comprising purifying one or more of the 1-SST enzyme, 1-FFT enzyme, and 6-SFT enzyme from the host cell.

15. A method of producing a fructan, comprising contacting sucrose with one or more of:
a) a sucrose:sucrose 1-fructosyltransferase (1-SST) enzyme comprising an amino acid sequence that is at least 90% identical to SEQ ID NO: 1 or SEQ ID NO: 24;
b) a fructan:fructan 1-fructosyltransferase (1-FFT) enzyme comprising an amino acid sequence that is at least 90% identical to SEQ ID NO: 7 or SEQ ID NO: 31; and
c) a sucrose:fructan-6-fructosyltransferase (6-SFT) enzyme comprising an amino acid sequence that is at least 90% identical to SEQ ID NO: 13 or SEQ ID NO: 38.

16. The method of claim 15, wherein the sucrose is contacted with two or more of a 1-SST enzyme, a 1-FFT enzyme, and a 6-SFT enzyme.

17. The method of claim 15, wherein the sucrose is contacted with a 1-SST enzyme, a 1-FFT enzyme, and a 6-SFT enzyme.

18. The method of any one of claims 15-17, wherein the fructan comprises a β(2,1) linkage, a β(2,6) linkage, or a combination thereof.

19. The method of any one of claims 15-18, wherein the fructan is a kestose, an inulin and/or a graminan.

20. The method of any one of claims 15-19, wherein the fructan has a degree of polymerization of at least 3.

21. The method of any one of claims 15-20, further comprising purifying the fructan.

22. The method of any one of claims 15-21, wherein the 1-SST enzyme, 1-FFT enzyme, and/or 6-SFT enzyme are secreted from one or more host cells.

23. The method of claim 22, wherein the one or more host cells are cultured in media containing sucrose, and wherein the sucrose is contacted with the 1-SST enzyme, 1-FFT enzyme, and/or 6-SFT enzyme in the media.

24. The method of claim 23, wherein the fructan is purified from the media.

25. The method of any one of claims 15-21, wherein the 1-SST enzyme, 1-FFT enzyme, and/or 6-SFT enzyme is a purified enzyme.

26. The method of any one of claims 19-25, wherein the kestose is 6-kestose.

27. The method of any one of claims 19-25, wherein the kestose is 1-kestose.

28. The method of any one of claims 15-25, wherein the fructan comprises a levan.

29. A method of producing a fructan, comprising:
a) contacting sucrose with a sucrose:sucrose 1-fructosyltransferase (1-SST) enzyme to produce kestose; and
b) contacting the kestose with a fructan:fructan 1-fructosyltransferase (1-FFT) enzyme and/or a sucrose:fructan-6-fructosyltransferase (6-SFT) enzyme to produce the fructan.

30. The method of claim 29, wherein the kestose produced in a) is purified and wherein the purified kestose is contacted with the 1-FFT enzyme and/or 6-SFT enzyme in b).

31. The method of claim 29 or 30, further comprising purifying the fructan produced in b).

32. The method of any one of claims 29-31, wherein the 1-SST enzyme, 1-FFT enzyme, and/or 6-SFT enzyme is secreted from one or more host cells.

33. The method of claim 32, wherein the one or more host cells is cultured in media containing sucrose, and wherein the sucrose is contacted with the 1-SST enzyme in the media.

34. The method of any one of claims 29-31, wherein the 1-SST enzyme, 1-FFT enzyme, and/or 6-SFT enzyme is a purified enzyme.

35. The method of any one of claims 29-34, wherein the fructan produced in b) is an inulin.

36. The method of any one of claims 29-35, wherein the fructan produced in b) is a branched inulin.

37. The method of any one of claims 29-34, wherein the fructan produced in b) is a graminan.

38. A host cell that comprises one or more heterologous polynucleotides encoding:
a) a sucrose:sucrose 1-fructosyltransferase (1-SST) enzyme comprising an amino acid sequence that is at least 90% identical to a sequence selected from SEQ ID NOs: 1-4 and 24-28;
b) a fructan:fructan 1-fructosyltransferase (1-FFT) enzyme comprising an amino acid sequence that is at least 90% identical to a sequence selected from SEQ ID NOs: 7-10 and 31-35; and/or
c) a sucrose:fructan-6-fructosyltransferase (6-SFT) enzyme comprising an amino acid sequence that is at least 90% identical to a sequence selected from SEQ ID NO: 13-21 and 38-52.

39. The host cell of claim 38, wherein at least two of the 1-SST, 1-FFT, and 6-SFT enzymes are encoded by the same heterologous polynucleotide.

40. A method of producing a fructan, comprising contacting sucrose with one or more of:
a) a sucrose:sucrose 1-fructosyltransferase (1-SST) enzyme comprising an amino acid sequence that is at least 90% identical to a sequence selected from SEQ ID NOs: 1-4 and 24-28;
b) a fructan:fructan 1-fructosyltransferase (1-FFT) enzyme comprising an amino acid sequence that is at least 90% identical to a sequence selected from SEQ ID NOs: 7-10 and 31-35; and
c) a sucrose:fructan-6-fructosyltransferase (6-SFT) enzyme comprising an amino acid sequence that is at least 90% identical to a sequence selected from SEQ ID NOs: 13-21 and 38-52.


SEQUENCE LISTING (63 sequences)

SEQ ID NO: 1 - protein, 651 aa, Artificial Sequence
  Synthetic
MRFPSIFTAVLFAASSALAAPVNTTTEDETAQIPAEAVIGYSDLEGDFDVAVLPFSNSTNNGLLFINTTIASIAAKEEGVSLEKREAEANLMRLRENDYPWTNDMLRWQRTGFHFQPGKNFQADPNAAMFYKGWYHFFYQYNPTGVAWDYTISWGHAVSKDLLHWNYLPMALRPDHWYDRKGVWSGYSTLLPDGRIVVLYTGGTKELVQVQNLAVPVNLSDPLLLEWKKSHVNPILVPPPGIEDHDFRDPFPVWYNESDSRWHVVIGSKDPEHYGIVLIYTTKDFVNFTLLPNILHSTKQPVGMLECVDLFPVATTDSRANQALDMTTMRPGPGLKYVLKASMDDERHDYYALGSFDLDSFTFTPDDETIDVGIGLRYDWGKFYASKTFYDQEKQRRVLWGYVGEVDSKRDDALKGWASLQNIPRTILFDTKTKSNLILWPVEEVESLRTINKNFNSIPLYPGSTYQLDVGEATQLDIVAEFEVDEKAIEATAEADVTYNCSTSGGAANRGVLGPFGLLVLANQELSEQTATYFYVSRGIDGNLRTHFCQDELRSSKAGAITKRVVGSTVPVLHGETWALRILVDHSIVESFAQRGRAVATSRVYPTEAIYSSARVFLFNNATDAIVTAKTVNVWHINSTYNHVFPGLVAP

SEQ ID NO: 2 - protein, 621 aa, Agave tequiliana
MASSTKDVEAPPTLDAPLLGPAAPRSRLRVAPVSLSVMAFLLVAIAAAVLYYNPGGVASNLMRLRENDYPWTNDMLRWQRTGFHFQPGKNFQADPNAAMFYKGWYHFFYQYNPTGVAWDYTISWGHAVSKDLLHWNYLPMALRPDHWYDRKGVWSGYSTLLPDGRIVVLYTGGTKELVQVQNLAVPVNLSDPLLLEWKKSHVNPILVPPPGIEDHDFRDPFPVWYNESDSRWHVVIGSKDPEHYGIVLIYTTKDFVNFTLLPNILHSTKQPVGMLECVDLFPVATTDSRANQALDMTTMRPGPGLKYVLKASMDDERHDYYALGSFDLDSFTFTPDDETIDVGIGLRYDWGKFYASKTFYDQEKQRRVLWGYVGEVDSKRDDALKGWASLQNIPRTILFDTKTKSNLILWPVEEVESLRTINKNFNSIPLYPGSTYQLDVGEATQLDIVAEFEVDEKAIEATAEADVTYNCSTSGGAANRGVLGPFGLLVLANQELSEQTATYFYVSRGIDGNLRTHFCQDELRSSKAGAITKRVVGSTVPVLHGETWALRILVDHSIVESFAQRGRAVATSRVYPTEAIYSSARVFLFNNATDAIVTAKTVNVWHINSTYNHVFPGLVAP

SEQ ID NO: 3 - protein, 591 aa, Arabidopsis thaliana
MAKLNRSNIGLSLLLSMFLANFITDLEASSHQDLNQPYRTGYHFQPLKNWMNGPMIYKGIYHLFYQYNPYGAVWDVRIVWGHSTSVDLVNWISQPPAFNPSQPSDINGCWSGSVTILPNGKPVILYTGIDQNKGQVQNVAVPVNISDPYLREWSKPPQNPLMTTNAVNGINPDRFRDPTTAWLGRDGEWRVIVGSSTDDRRGLAILYKSRDFFNWTQSMKPLHYEDLTGMWECPDFFPVSITGSDGVETSSVGENGIKHVLKVSLIETLHDYYTIGSYDREKDVYVPDLGFVQNESAPRLDYGKYYASKTFYDDVKKRRILWGWVNESSPAKDDIEKGWSGLQSFPRKIWLDESGKELLQWPIEEIETLRGQQVNWQKKVLKAGSTLQVHGVTAAQADVEVSFKVKELEKADVIEPSWTDPQKICSQGDLSVMSGLGPFGLMVLASNDMEEYTSVYFRIFKSNDDTNKKTKYVVLMCSDQSRSSLNDENDKSTFGAFVAIDPSHQTISLRTLIDHSIVESYGGGGRTCITSRVYPKLAIGENANLFVFNKGTQSVDILTLSAWSLKSAQINGDLMSPFIEREESRSPNHQF

SEQ ID NO: 4 - protein, 628 aa, Asparagus officinalis
MASPSDLESPPTLSAQLLESRPPRSKLRLVALTLTAAAFLVALALFLADGSASRFVSGLARKLRSDPIKEHDYPWTNEMLTWQRSGFHFQPAKNFQSDPNAAMYYKGWYHFFYQYNPTGTAWDYTISWGHAVSRDLIHWLHLPMAMVPDHWYDAKGVWSGYSTLLPDGRVIVLYTGGTPELVQVQNLAVPADASDPLLLKWKKSSVNPILVPPPGIGTSDFRDPFPIWYNETDSNWHVLIGSKDSNHHGIVLLYKTKDFFNFTLLPSLLHTSTQSVGMFECVDLYPVATGGPLSNRGLEMSVDLSNGGIKHVLKASMDEERHDYYAIGTFDLDSFKWTPDDPSIDVGVGLRYDWGKFYASKTFFDTEKQRRILWGYVGEVDSKDDDKMKGWATLQNIPRTILLDTKTQSNLIIWPVEEVEDLRTDGNIFNDIKIGAGSSVQLDIGAASQLDIEAEFELDNSALDGAIEADVTYNCSTSGGAANRGLLGPFGLLVLANQDLTEQTATYFYVSRGTDGDLRTHFCQDELRSSKAGDIVKRVVGSVVPVLHGETWSLRILVDHSIIESFAQRGRAVATSRVYPTEAIYNKARLFLFNNATDAKVTAKSVKIWHMNSTHNHPFPGLESLFES

SEQ ID NO: 5 - DNA, 1953 bp, Artificial Sequence
  Synthetic
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctaacttgatgcgtttaagagagaatgattatccctggactaacgacatgctaagatggcaacgcacgggatttcacttccagcctggtaaaaacttccaagccgacccaaatgcagctatgttttacaagggctggtaccatttcttttatcaatacaacccgaccggtgtggcttgggattacacaatctcctggggtcacgctgtcagtaaggatttgctgcattggaattatcttccaatggccttgaggcctgaccactggtacgatagaaaaggtgtttggagcggttactctactttattgccagacggtagaattgttgtcttgtacaccggtggaactaaggaattagttcaagtccaaaacttggctgtcccagtaaacctttctgacccattgctattggaatggaagaagtcacacgttaacccaatactcgttccacctccggggatcgaagatcatgatttccgagatccattcccagtgtggtataatgaatctgactcgcggtggcacgttgtaattggttccaaagatccagagcactatggtattgtcttgatctacactaccaaggacttcgttaactttacgttattaccaaacatattgcattccaccaagcagccggttggtatgctggaatgtgtagacttgttcccagttgctacaactgattctcgtgcaaatcaagctttggatatgactaccatgaggcccggtcctgggctcaaatatgtgttaaaggcgagtatggatgacgaaagacacgattactacgccctaggtagctttgacttggactcgttcacttttacaccagatgatgaaaccattgacgtcggtattggtcttagatacgactggggcaagttctacgcgtccaagactttttacgaccaagaaaaacaaagaagagttttgtggggatacgtcggtgaagttgactcgaagcgtgatgatgctctgaaaggttgggcttctttgcaaaatatcccacgtacaatcttgttcgacaccaaaaccaagtccaacctaattttgtggccagttgaagaagtcgagtctttaagaactattaacaagaatttcaattcaatccctttgtatcctggttctacttaccagcttgatgtgggtgaagctacccaattggatattgtggccgagttcgaagtcgatgaaaaggctattgaagctactgccgaagctgatgttacatataactgctccacctccggtggtgcagctaatagaggggttttgggtccattcggtttgttagttttagctaaccaagagttgtctgaacaaactgctacttacttctatgtctctcgcggcatagatggtaacttaagaacacatttttgtcaagacgaactgcgatcttccaaggctggtgccatcactaagcgggtagttggttctaccgtcccagttctacatggcgaaacctgggccttgagaattttggtcgatcactcaatcgtagagtcttttgcacagagaggtagagctgttgccacgagtagagtctatcctacagaagcaatttatagctcagctagagtctttctattcaacaatgccactgacgctattgttaccgctaagacagtaaacgtttggcacatcaactccacctacaatcatgtttttccgggtctggtcgctcca

SEQ ID NO: 6 - protein, 654 aa, Festuca arundinacea
MESSAVVPGTTAPLLPYAYAPLPSSADDARENQSSGGVRWRVCAAVLAASALAVLIVVGLLAGGRVDRGPAGGDVASAAVPAVPMEIPRSRGKDFGVSEKASGAYSADGGFPWSNAMLQWQRTGFHFQPEKHYMNDPNGPVYYGGWYHLFYQYNPKGDSWGNIAWAHAVSKDMVNWRHLPLAMVPDQWYDSNGVLTGSITVLPDGQVILLYTGNTDTLAQVQCLATPADPSDPLLREWIKHPANPILYPPPGIGLKDFRDPLTAWFDHSDNTWRTVIGSKDDDGHAGIILSYKTKDFVNYELMPGNMHRGPDGTGMYECIDLYPVGGNSSEMLGGDDSPDVLFVLKESSDDERHDYYALGRFDAAANIWTPIDQELDLGIGLRYDWGKYYASKSFYDQKKNRRIVWAYIGETDSEQADITKGWANLMTIPRTVELDKKTRTNLIQWPVEELDTLRRNSTDLSGITVDAGSVIRLPLHQGAQIDIEASFQLNSSDVDALTEADVSYNCSTSGAAVRGALGPFGLLVLANGRTEQTAVYFYVSKGVDGALQTHFCHDESRSTQAKDVVNRMIGSIVPVLDGETFSVRVLVDHSIVQSFAMGGRITATSRAYPTEAIYAAAGVYLFNNATGATVTAERLVVYEMASADNHIFTNDDL

SEQ ID NO: 7 - protein, 631 aa, Artificial Sequence
  Synthetic
MRFPSIFTAVLFAASSALAAPVNTTTEDETAQIPAEAVIGYSDLEGDFDVAVLPFSNSTNNGLLFINTTIASIAAKEEGVSLEKREAEASSVQPSAAERLTWERTAFHFQPAKNFIYDPNGPLFHMGWHHLFYQYNPYAPVWGNMSWGHAVSKDMINWFELPVALVPTEWYDIEGVLSGSTTALPNGQIFALYTGNANDFSQLQCKAVPVDVSDPLLVKWVKYDGNPILYTPPGIGLKDYRDPSTVWTGPDGKHRMIMGTKRGTTGLVLVYHTTDFTNYVMLDEPLHSVPNTDMWECVDLFPVSTTNDSALDIAAYGSGIKHVLKESWEGHAMDFYSIGTYDAINDKWTPDNPELDVGIGLRCDYGRFFASKSLYDPLKKRRVTWGYVAESDSADQDVSRGWATIYNVARTIVLDRKTGTHLLQWPVEELESLRSNVREFKEMTLEPGSIVPLDIGSATQLDIIATFEVDQEALKATSDANDEYACTTSSGAAERGSFGPFGIAVLADGTLSELTPVYFYIAKNTKGGVDTHFCTDKLRSSLDYDSEKVVYGSTIPVLDGEQITMRVLVDHSVVEGFAQGGRTVITSRVYPTKAIYEGAKLFVFNNATTTNVKATLNVWQMSHALIQPYPF

SEQ ID NO: 8 - protein, 617 aa, Acrtium lappa
MKTTEPLTDLEHAPNHTPLLDHPQPPPATVSKRLLIRVLSSITFVSLFFVSAFLLILLNQHESSYTDDNLAPLDRSSVQPSAAERLTWERTAFHFQPAKNFIYDPNGPLFHMGWHHLFYQYNPYAPVWGNMSWGHAVSKDMINWFELPVALVPTEWYDIEGVLSGSTTALPNGQIFALYTGNANDFSQLQCKAVPVDVSDPLLVKWVKYDGNPILYTPPGIGLKDYRDPSTVWTGPDGKHRMIMGTKRGTTGLVLVYHTTDFTNYVMLDEPLHSVPNTDMWECVDLFPVSTTNDSALDIAAYGSGIKHVLKESWEGHAMDFYSIGTYDAINDKWTPDNPELDVGIGLRCDYGRFFASKSLYDPLKKRRVTWGYVAESDSADQDVSRGWATIYNVARTIVLDRKTGTHLLQWPVEELESLRSNVREFKEMTLEPGSIVPLDIGSATQLDIIATFEVDQEALKATSDANDEYACTTSSGAAERGSFGPFGIAVLADGTLSELTPVYFYIAKNTKGGVDTHFCTDKLRSSLDYDSEKVVYGSTIPVLDGEQITMRVLVDHSVVEGFAQGGRTVITSRVYPTKAIYEGAKLFVFNNATTTNVKATLNVWQMSHALIQPYPF

SEQ ID NO: 9 - protein, 622 aa, Taraxacum kok-saghyz
MKTIEPFSDVENAPNSTPLLNHPEPPRAAVRKQSFVRVLSSITLVSLFFVLAFVLIVLNQQDSTTTVANSAPPGATVPEKSSVKHSQSDRLRWERTAYHFQPAKNFIYDPNGPLFHMGWYHLFYQYNPYAPIWGNMSWGHAVSKDMIHWFELPVAIVPTEWYDIEGVLSGSTTALPNGQIFALYTGNAKDFSQLQCKAVPLNASDPLLVEWVKYEDNPILYIPPGIGPKDYRDPSTVWTGPDGKHRMIMGTKQNGTGMVHVYHTTDFINYVLLDEPLHSVPNTDMWECVDFYPVSTINDSALDIAAYGSDIKHVIKESWEGHGMDLYSIGTYDAYKDKWTPDNPEFDVGIGLRVDYGRFFASKSLYDPLKKRRVTWGYVAESDSSDQDLNRGWATIYNVGRTVVLDRKTGTHLLHWPVEEIESLRSNVREFNEIELVPGSIIPLDIGMATQLDIVATFKVDPEALMAKSDINSEYGCTTSSGATQRGSLGPFGIVVLADVALSELTPVYFYIAKNIDGGLVTHFCTDKLRSSLDYDGERVVYGSTVPVLDGEELTMRLLVDHSVVEGFAQGGRTVMTSRVYPTNAIYEEAKIFLFNNATGASVKASLKIWQMGSASIQAYPF

SEQ ID NO: 10 - protein, 622 aa, Taraxacum brevicorniculatum
MKTIEPFSDVENAPNSTPLLNHPEPSRAAVRKQSFVRVLSSITLVSLFFVLAFVLIVLNQQDSTNTVANSAPPGATVPEKSSVKHSQSDRLRWERTAYHFQPAKNFIYDPNGPLFHMGWYHLFYQYNPYAPIWGNMSWGHAVSKDMIHWFELPVAMVPTEWYDIEGVLSGSTTALPNGQIFALYTGNAKDFSQLQCKAVPLNASDPLLVDWVKYEDNPILYIPPGIGPKDYRDPSTVWTGPDGKHRMIMGTKQNGTGMVHVYHTTDFINYVLLDEPLHSVPNTDMWECVDFYPVSTINDSALDIAAYGSDIKHVIKESWEGHGMDLYSIGTYDAYKDKWTPDNPELDVGIGLRVDYGRLFASKSLYDPLKKRRVTWGYVGESDSPDQDINRGWATIYNVGRTVVLDRKTGTHLLHWPVEEIESLRSNVREFNEIELVPGSIIPLDIGMATQLDIVATFKVDPEALMAKSDINSEYGCTTSSGATQRGSLGPFGIVVLADLALSELTPLYFYIAKNTDGGLVTHFCTDKLRSSLDYDGERVVYGGTVPVLDGEELTMRLLVDHSVVEGFAQGGRTVITSRVYPTNAIYEEAKIFLFNNATGASVKASLKIWQMGSASIQAYPF

SEQ ID NO: 11 - DNA, 1896 bp, Artificial Sequence
  Synthetic
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctagttccgttcaaccttctgccgctgaacgtttaacctgggagagaactgcattccattttcagccagctaaaaatttcatttatgatccaaacggaccgctgtttcacatgggctggcaccatcttttctaccaatacaacccctacgctccagtctggggtaatatgagctggggtcacgcggtgtcaaaggacatgataaactggttcgaattgccagtagccttagttccaacggaatggtatgatattgaaggtgttctatctggttctactacagctttgcctaatgggcaaatctttgctttgtacaccggtaacgccaacgacttctcccaattgcaatgtaaggctgtcccagttgacgtgtcggatccattattggtcaaatgggttaagtatgacggtaatccgatcttgtacactccacctggaatcggtctgaaggattatagagatccatctaccgtctggactggtccagacggtaagcataggatgattatgggtacaaagagaggtaccactggcttggttttagtttaccacacaacggatttcactaactacgtcatgttggacgaaccactccactcagtaccaaacactgacatgtgggaatgcgttgatctttttccggtcagcaccaccaatgatagtgctttggacatcgcggcttatggttccggtattaaacatgttttgaaagagtcttgggaaggtcacgcaatggatttctactccattgggacttacgatgctataaacgacaagtggactcctgacaacccagaactagacgtcggtattggtttgagatgtgattacggtagatttttcgcatctaagtccctatacgatcctttaaagaaacggagagttacctggggatatgtcgccgaatctgattcagccgaccaagacgtgtctcgcggttgggctacaatctataatgttgcaaggactattgttttagaccgtaagaccggcactcatctgcttcagtggccagtcgaagaattggagtcccttagatcgaacgtgagagaatttaaggaaatgaccttggaaccaggttccatcgttccattggatataggttctgctactcaattggatattatcgctacgttcgaagttgaccaagaagctttgaaagctacctctgacgctaacgacgaatacgcctgtacaacatcttcaggtgctgcggagcgtggttcgttcggtcccttcggtatcgctgtcctcgccgatggtaccttgtccgaactgactccagtatacttctacattgctaaaaatactaagggcggggtcgatacgcacttttgtactgataagttgagaagctctttagactatgacagtgaaaaggttgtctacgggagtaccattccagttttagatggtgaacaaatcactatgagagttctcgtcgatcattccgttgtggaaggttttgcccagggtggtagaactgtaattaccagtagagtttaccctaccaaggctatatacgaaggtgccaagttgtttgtattcaataacgctacaactacaaatgttaaggcaacgttgaatgtatggcaaatgtcacacgccctcatccaaccatacccattctaa

SEQ ID NO: 12 - protein, 608 aa, Echinops ritro
EPFSDLEHAPNHTPLLDRPKTPPAAVSHRLLIRVLSTITVVSLFFVAAFLLVLNQQDSGNNPLPQDPPPQPSAADRLRWERTAYHYQPAKNFMYDPNGPIFHMGWYHLFYQYNPYSVFWGNMTWGHAVSKDMINWFELPVALAPVEWYDIEGVLSGSTTVLPTGEIFALYTGNANDFSQLQCKAVPVNTSDPLLIDWVRYEGNPILYTPPGVGLTDYRDPSTVWTGPDNIHRMIIGTRRNNTGLVLVYHTKDFINYELLDEPLHSVPDSGMWECVDLYPVSTMNDTALDVAAYGSGIKHVLKESWEGHAKDFYSIGTYDAINDKWWPDNPELDLGMGWRCDYGRFFASKTLYDPLKKRRVTWGYVAESDSGDQDRSRGWSNIYNVARTVMLDRKTGTNLLQWPVEEIESLRSKVHEFNEIELQPGSIIPLEVGSTTQLDIVATFEVNKDAFEETNVNYNEYGCTSSKGASQRGRLGPFGIIVLADGNLLELTPVYFYIAKNNDGSLTTHFCTDKLRSSFDYDDEKVVYGSTVPVLEGEKLTIRLMVDHSIIEGFAQGGRTVITSRVYPTKAIYDTAKLFLFNNATDITVKASLKVWHMASANIQMYPF

SEQ ID NO: 13 - protein, 638 aa, Artificial Sequence
  Synthetic
MRFPSIFTAVLFAASSALAAPVNTTTEDETAQIPAEAVIGYSDLEGDFDVAVLPFSNSTNNGLLFINTTIASIAAKEEGVSLEKREAEAVPGKLESNADVEWQRSAYHFQPDKNFISDPDGPMYHMGWYHLFYQYNPESAIWGNITWGHSVSRDMINWFHLPFAMVPDHWYDIEGVMTGSATVLPNGQIIMLYTGNAYDLSQLQCLAYAVNSSDPLLLEWKKYEGNPILFPPPGVGYKDFRDPSTLWMGPDGEWRMVMGSKHNETIGCALVYRTTNFTHFELNEEVLHAVPHTGMWECVDLYPVSTTHTNGLEMKDNGPNVKYILKQSGDEDRHDWYAIGTFDPEKDKWYPDDPENDVGIGLRYDYGKFYASKTFYDQHKKRRVLWGYVGETDPPKSDLLKGWANILNIPRSVVLDTQTETNLIQWPIEEVEKLRSKKYDEFKDVELRPGSLIPLEIGTATQLDISATFEIDEKKLESTLEADVLFNCTTSEGSVGRGVLGPFGIVVLADANRSEQLPVYFYIAKDTDGTSRTYFCADESRSSKDKDVGKWVYGSSVPVLEGENYNMRLLVDHSIVEGFAQGGRTVVTSRVYPTMAIYGAAKIFLFNNATGISVKASLKIWKMAEAQLDPFPLSGWSS

SEQ ID NO: 14 - protein, 640 aa, Cichorium intybus
MASSTTATTPLILRDETQIRPQLAGSSVGRRLSMAKILSGILVFVLVICALVAVIHDQSQQTMATNNHQGGDKPTSAATFTAPLPQVGLKRVPGKLESNADVEWQRSAYHFQPDKNFISDPDGPMYHMGWYHLFYQYNPESAIWGNITWGHSVSRDMINWFHLPFAMVPDHWYDIEGVMTGSATVLPNGQIIMLYTGNAYDLSQLQCLAYAVNSSDPLLLEWKKYEGNPILFPPPGVGYKDFRDPSTLWMGPDGEWRMVMGSKHNETIGCALVYRTTNFTHFELNEEVLHAVPHTGMWECVDLYPVSTTHTNGLEMKDNGPNVKYILKQSGDEDRHDWYAIGTFDPEKDKWYPDDPENDVGIGLRYDYGKFYASKTFYDQHKKRRVLWGYVGETDPPKSDLLKGWANILNIPRSVVLDTQTETNLIQWPIEEVEKLRSKKYDEFKDVELRPGSLIPLEIGTATQLDISATFEIDEKKLESTLEADVLFNCTTSEGSVGRGVLGPFGIVVLADANRSEQLPVYFYIAKDTDGTSRTYFCADESRSSKDKDVGKWVYGSSVPVLEGENYNMRLLVDHSIVEGFAQGGRTVVTSRVYPTMAIYGAAKIFLFNNATGISVKASLKIWKMAEAQLDPFPLSGWSS

SEQ ID NO: 15 - protein, 616 aa, Aegilops searsii
MGSHGKPPLPYAYKPLPSDADGERTGCTRWRVCATALTASAMVVVVVGATLLAGFRVDQAVDEEAAGGFPWSNEMLQWQRSGYHFQTAKNYMSDPNGLMYYRGWYHMFFQYNPVGTDWDDGMEWGHAVSRNLVQWRTLPIAMVADQWYDILGVLSGSMTVLPNGTVIMIYTGATNASAVEVQCIATPADPNDPLLRRWTKHPANPVIWSPPGVGTKDFRDSMTAWYDESDDTWRTLLGSKDDNNGHHDGIAMMYKTKDFLNYELIPGILHRVERTGEWECIDFYPVGHRTSDNSSEMLHVLKASMDDERHDYYSLGTYDSAANRWTPIDPELDLGIGLRYDWGKFYASTSFYDPAKKRRVLMGYVGEVDSKRADVVKGWASIQSVPRTIALDEKTRTNLLLWPVEEIETLRLNATQLSDVTLNTGSVIHIPLRQGTQLDIEATFHLDASAVAALNEADVGYNCSSSGGAVNRGALGPFGLLVLAAGDRRGEQTAVYFYVSRGLDGGLHTSFCQDELRSSRAKDVTKRVIGSTVPVLDGEAFSMRVLVDHSIVQGFAMGGRTTMTSRVYPMEAYQEAKVYLFNNATGASVTAERLVVHDMDSAHNQLSNMDDYSYVQ

SEQ ID NO: 16 - protein, 616 aa, Unknown
  Triticum turgidum subsp. durum
MGSHGKPPLPYAYKPLPSDADGERTGCTRWRVCAVALTASAMVVVVVGATLLAGFRVDQAVDEEAAGGFPWSNEMLQWQRSGYHFQTAKNYMSDPNGLMYYRGWNHMFFQYNPVGTDWDDGMEWGHAVSRNLVQWRTLPIAMVADQWYDILGVLSGSMTVLPNGTVIMIYTGATNASAVEVQCIATPADPTDPLLRRWTKHPANPVIWSPPGVGTKDFRDPMTAWYDESDDTWRTLLGSKDDNNGHHDGIAMMYKTKDFLNYELIPGILHRVERTGEWECIDFYPVGRRTSDNSSEMLHVLKASMDDERHDYYSLGTYDSAANRWTPIDPELDLGIGLRYDWGKFYASTSFYDPAKKRRVLMGYVGEVDSKRADVVKGWASIQSVPRTIALDEKTRTNLLLWPVEEIETLRLNATELSDVTLNTGSVIHIPLRQGTQLDIEATFHLDASAVAAFNEADVGYNCSSSGGAVNRGALGPFGLLVLAAGDRRGEQTAVYFYVSRGLDGGLHTSFCQDELRSSRAKDVTKRVIGSTVPVLDGEAFSMRVLVDHSIVQGFAMGGRTTMTSRVYPMEAYQEAKVYLFNNATGASVTAERLVVHEMDSAHNQLSNMDDHSYVQ

SEQ ID NO: 17 - protein, 648 aa, Unknown
  Triticum turgidum subsp. durum
MESSRGILIPGTPPLPYAYEPLPSSLTDANGQEDRRITGGVRWRAWAAVLAVGALVVAAAVFGASRVDRDAVASSVPATAEHGVLEKASGPYSASGGFPWSNAMLQWQRTGYHFQPEKNYQNDPNGPVYYKGWYHFFYQHNPGGTGWGNISWGHAVSRDMVHWRHLPLAMVPEHWYDIEGVLTGSITVLPDGRVILLYTGNTETFAQVTCLAEAADPSDPLLREWAKHPANPVVYPPPGIGMKDYRDPTTAWFDNSDNTWRIIIGSKNDTDHSGIVFTYKTKDFVSYELIPGYLYRGPAGTGMYECIDLFAVGGGRAASDMYNSTAEDVLYVLKESSDDDRRDYYALGRFDAAANTWTPIDTERELGVALRYDYGRYDTSKSFYDPVKQRRIVWGYVVETDSWSADAAKGWANLQSIPRTVELDEKTRTNLVQWPVGELNTLRINTTDLSDITVGAGSVDSLPLHQTSQLDIEASFRINASTIEALNEVDVGYNCTMTSGAATRGALGPFGILVLANVALTEQTAVYFYVSKGLDGGLRTHFCHDELRSTHATDVAKEVVGSTVPVLDGEDFSVRVLVDHSIVQSFVMGGRMTATSRAYPTEAIYAAAGVYLFNNATGASITAEKLVVHDMDSSYNRIFTDEDLLVLD

SEQ ID NO: 18 - protein, 556 aa, Triticum aestivum
MANAFPWSNAMLQWQRTGFHFQPDKYYQNDPNGPVYYGGWYHFFYQYNPSGSVWEPQIVWGHAVSKDLIHWRHLPPALVPDQWYDIKGVLTGSITVLPDGKVILLYTGNTETFAQVTCLAEPADPSDPLLREWVKHPANPVVFPPPGIGMKDFRDPTTAWYDESDGTWRTIIGSKNDSDHSGIVFSYKTKDFISYELMPGYMYRGPKGTGEYECIDLYAVGGGRKASDMYNSTAEDVLYVLKESSDDDRHDWYSLGRFDAAANKWTPIDTELELGVGLRYDWGKYYASKSFYDPVKKRRVVWAYVGETDSERADITKGWANLQSIPRTVELDEKTRTNLIQWPVEELNTLRINTTDLSGITVGAGSVAFLPLHQTAQLDIEATFRIDASAIEALNEADVSYNCTTSRGAATRGALGPFGLLVLANHALTEQTGVYFYVSKGLDGGLRTHFCHDELRSSHASDVVKRVVGSTVPVLDGEDFSVRVLVDHSIVQSFAMGGRLTATSRAYPTEAIYAAAGVYMFNNATGTSVTAEKLVVHDMDSSYNHIYTDGDLVVVD

SEQ ID NO: 19 - protein, 623 aa, Allium cepa
MESRDIESSPALNAPLLQASPPIKSSKLKVALLATSTSVLLLIAAFFAVKYSVFDSGSGLLKDDPPSDSEDYPWTNEMLKWQRTGYHFQPPNHFMADPNAAMYYKGWYHFFYQYNPNGSAWDYSISWGHAVSKDMIHWLHLPVAMVPDHWYDSKGVWSGYATTLPDGRIIVLYTGGTDQLVQVQNLAEPADPSDPLLIEWKKSNGNPILMPPPGVGPHDFRDPFPVWYNESDSTWHMLIGSKDDNHYGTVLIYTTKDFETYTLLPDILHKTKDSVGMLECVDLYPVATTGNQIGNGLEMKGGSGKGIKHVLKASMDDERHDYYAIGTFDLESFSWVPDDDTIDVGVGLRYDYGKFYASKTFYDQEKKRRILWGYVGEVDSKADDILKGWASVQNIARTILFDAKTRSNLLVWPVEELDALRTSGKEFNGVVVEPGSTYHLDVGTATQLDIEAEFEINKEAVDAVVEADVTYNCSTSDGAAHRGLLGPFGLLVLANEKMTEKTATYFYVSRNVDGGLQTHFCQDELRSSKANDITKRVVGHTVPVLHGETFSLRILVDHSIVESFAQKGRAVATSRVYPTEAIYDSTRVFLFNNATSATVTAKSVKIWHMNSTHNHPFPGFPAP

SEQ ID NO: 20 - protein, 621 aa, Agave tequilana
MASSTKDVEAPPTLDAPLLGSAAPRSRLRVAAVSLSVMAFLLVAIAAAVLYYNPGGVASNLMRLRENDYPWTNDMLRWQRTGFHFQPEKNFQADPNAAMFYKGWYHFFYQYNPTGVAWDYTISWGHAVSKDLLHWNYLPMALRPDHWYDRKGVWSGYSTLLPDGRIVVLYTGGTKELVQVQNLAVPVNLSDPLLLEWKKSHVNPILVPPPGIEDHDFRDPFPVWYNESDSRWHVVIGSKDPEHYGIVLIYTTKDFVNFTLLPNILHSTKQPVGMLECVDLFPVATTDSRANQALDMTTMRPGPGLKYVLKASMDDERHDYYALGSFDLDSFTFTPDDETIDVGVGLRYDWGKFYASKTFYDQEKHRRVLWGYVGEVDSKRDDALKGWASLQNIPRTILFDTKTKSNLILWPVEEVESLRTINKNFNSIPLYPGSTYQLDVGEATQLDIVAEFEVDEKAIEATAEADVTYNCSTSGGAANRGVLGPFGLLVLANQELSEQTATYFYVSRGIDGNLRTHFCQDELRSSKAGAITKRVVGSTVPVLHGETWALRILVDHSIVESFAQRGRAVATSRVYPTEAIYSSARVFLFNNATDAIVTAKTVNVWHMNSTYNHVFPGLVAP

SEQ ID NO: 21 - protein, 623 aa, Allium cepa
MESRDIESSPALNAPLLQTSPPIKSSKLKVALLATSTSVLLLIAAFFAVKYSVFDSGSGLLKDDPPSDSEDYPWTNEMLKWQRTGYHFQPPNHFMADPNAAMYYKGWYHFFYQYNPNGSAWDYSISWGHAVSKDMIHWLHLPVAMVPDHWYDSKGVWSGYATTLPDGRIIVLYTGGTDQLVQVQNLAEPADPSDPLLIEWKKSNGNPILMPPPGVGPHDFRDPFPVWYNESDSTWHMLIGSKDDNHYGTVLIYTTKDFETYTLLPDILHKTKDSVGMLECVDLYPVATTGNQIGNGLEMKGGSGKGIKHVLKASMDDERHDYYAIGTFDLESFSWVPDDDTIDVGVGLRYDYGKFYASKTFYDQEKKRRILWGYVGEVDSKADDILKGWASVQNIARTILFDAKTRSNLLVWPVEELDALRTSGKEFNGVVVEPGSTYHLDVGTATQLDIEAEFEINKEAVDAVVEADVTYNCSTSDGAAHRGLLGPFGLLVLANEKMTEKTATYFYVSRNADGGLQTHFCQDELRSSKANDITKRVVGHTVPVLHGETFSLRILVDHSIVESFAQKGRAVATSRVYPTEAIYDSTRVFLFNNATSATVTAKSVKIWHMNSTHNHPFPGFPAP

SEQ ID NO: 22 - DNA, 1917 bp, Artificial Sequence
  Synthetic
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgtacccggtaaattagaatcgaatgccgatgtcgagtggcaacgttctgcataccattttcagccagacaagaacttcatatccgatcctgacggcccaatgtatcacatgggatggtaccacctattctaccaatataacccggaatcagctatttgggggaatatcacttggggtcatagtgtgtctagggacatgattaactggtttcacttgccattcgctatggttccagatcattggtacgacatcgaaggtgttatgaccggtagcgctacggttcttcctaacggtcaaatcattatgttgtatactggtaatgcgtacgatttgtctcaattgcaatgcttagcttatgccgtcaactcctcagatccactactcttggaatggaagaagtacgaaggtaatccaatattgttcccaccacccggtgtcggttacaaagactttagagatccttccaccttatggatgggcccagacggcgaatggagaatggttatgggtagtaagcacaacgagacaatcggatgtgctttggtctatcgaactaccaatttcactcactttgaacttaacgaagaagttttacatgctgtaccacacacaggaatgtgggaatgtgtggatctctacccggtcagcacgacccatactaacgggttggaaatgaaggacaatggtccaaacgttaaatatattttaaagcaatctggtgatgaggatagacacgactggtacgccattggtacattcgatccagaaaaggacaaatggtaccctgatgacccagagaatgacgttggtatcggtttgagatacgactatgggaagttctatgccagtaagactttttacgatcaacataaaaagcggagagtattgtggggttacgttggtgaaactgatccaccaaagtcggatctattgaaaggttgggctaacattctcaacatccctagatcagtcgttttggatacccagacagagactaatttgattcaatggccaatcgaagaagttgaaaaacttagatccaagaagtacgacgaatttaaggacgtcgaactgcgtcctggttctttgattccattggaaatcggtaccgctacccaattggatatatctgcaactttcgaaattgatgaaaagaaactggagtctactttagaagctgacgttttattcaactgtacaacttcagaaggttccgtcggtagaggtgttctaggccctttcggtatcgttgtcttggctgatgctaacagatccgaacaattgccagtttacttctacattgcaaaggacaccgatggtacttctcgcacctatttctgtgctgacgaatctcgttcttcgaaggataaggatgtgggtaagtgggtttacggatcttccgtaccagtcctggagggtgaaaactataatatgagattgctcgtcgatcattcgattgtagaaggttttgcccaagggggtagaaccgttgtcacctctcgcgtttatccaacgatggcaatctacggtgccgctaagatatttttgttcaacaatgctaccggtatttcagtgaaggctagtttaaaaatctggaagatggctgaggcccaattggaccccttcccactttccggttggagcagttaa

SEQ ID NO: 23 - protein, 623 aa, Phleum pratense
MAPPQAIANGAPAPLPYAYARLPSSGDEKQDQSKSGGARYCRACVAGVAALLIVAGALAGARVGLGGIYDDADAFAWNNSMLQWQRAGFHFQTEKNFMSDPNGPVYYRGYYHLFYQYNMKGVVWDDGIVWGHVVSRDLVHWRHLPIAMVPDHWYDSMGVLSGSITVLQNGSLVMIYTGVFSKTTDRSGMMEVQCLAVPADPNDPLLRSWTKHPANPVLVHPPGIKDMDFRDPTTAWFDESDSTYRTVIGTKDDHHGSHAGFAMVYKTKDFLSFQRIPGILHSVEHTGMWECMDFYPVGGGDNSSSEVLYVIKASMDDERHDYYALGMYDAAANTWTPLDQELDLGIGLRYDWGKLYASTTFYDPAKRRRVMLGYVGETDSRRSDEAKGWASIQSIPRTVALDEKTRTNLLLWPVEEIETLRLNATEFNDINIDTGSVFHLPIRQGNQLDIEASFRLDASAVAAINEADVGYNCSSSGGAATRGALGPFGLLVLAAEGIGEQTAVYFYVSRGLDGGLRTSFCNDELRSSWARDVTKRVVGSTVPVLNGETLSMRVLVDHSIVQSFAMGGRVTATSRVYPTEAIYAAAGVYLFNNATNASVTAERIIVHEMDSIDNNQIFLIDDL

SEQ ID NO: 24 - protein, 562 aa, Artificial Sequence
  Synthetic
NLMRLRENDYPWTNDMLRWQRTGFHFQPGKNFQADPNAAMFYKGWYHFFYQYNPTGVAWDYTISWGHAVSKDLLHWNYLPMALRPDHWYDRKGVWSGYSTLLPDGRIVVLYTGGTKELVQVQNLAVPVNLSDPLLLEWKKSHVNPILVPPPGIEDHDFRDPFPVWYNESDSRWHVVIGSKDPEHYGIVLIYTTKDFVNFTLLPNILHSTKQPVGMLECVDLFPVATTDSRANQALDMTTMRPGPGLKYVLKASMDDERHDYYALGSFDLDSFTFTPDDETIDVGIGLRYDWGKFYASKTFYDQEKQRRVLWGYVGEVDSKRDDALKGWASLQNIPRTILFDTKTKSNLILWPVEEVESLRTINKNFNSIPLYPGSTYQLDVGEATQLDIVAEFEVDEKAIEATAEADVTYNCSTSGGAANRGVLGPFGLLVLANQELSEQTATYFYVSRGIDGNLRTHFCQDELRSSKAGAITKRVVGSTVPVLHGETWALRILVDHSIVESFAQRGRAVATSRVYPTEAIYSSARVFLFNNATDAIVTAKTVNVWHINSTYNHVFPGLVAP

SEQ ID NO: 25 - protein, 648 aa, Artificial Sequence
  Synthetic
MRFPSIFTAVLFAASSALAAPVNTTTEDETAQIPAEAVIGYSDLEGDFDVAVLPFSNSTNNGLLFINTTIASIAAKEEGVSLEKREAEADLNQPYRTGYHFQPLKNWMNGPMIYKGIYHLFYQYNPYGAVWDVRIVWGHSTSVDLVNWISQPPAFNPSQPSDINGCWSGSVTILPNGKPVILYTGIDQNKGQVQNVAVPVNISDPYLREWSKPPQNPLMTTNAVNGINPDRFRDPTTAWLGRDGEWRVIVGSSTDDRRGLAILYKSRDFFNWTQSMKPLHYEDLTGMWECPDFFPVSITGSDGVETSSVGENGIKHVLKVSLIETLHDYYTIGSYDREKDVYVPDLGFVQNESAPRLDYGKYYASKTFYDDVKKRRILWGWVNESSPAKDDIEKGWSGLQSFPRKIWLDESGKELLQWPIEEIETLRGQQVNWQKKVLKAGSTLQVHGVTAAQADVEVSFKVKELEKADVIEPSWTDPQKICSQGDLSVMSGLGPFGLMVLASNDMEEYTSVYFRIFKSNDDTNKKTKYVVLMCSDQSRSSLNDENDKSTFGAFVAIDPSHQTISLRTLIDHSIVESYGGGGRTCITSRVYPKLAIGENANLFVFNKGTQSVDILTLSAWSLKSAQINGDLMSPFIEREESRSPNHQF

SEQ ID NO: 26 - protein, 559 aa, Artificial Sequence
  Synthetic
DLNQPYRTGYHFQPLKNWMNGPMIYKGIYHLFYQYNPYGAVWDVRIVWGHSTSVDLVNWISQPPAFNPSQPSDINGCWSGSVTILPNGKPVILYTGIDQNKGQVQNVAVPVNISDPYLREWSKPPQNPLMTTNAVNGINPDRFRDPTTAWLGRDGEWRVIVGSSTDDRRGLAILYKSRDFFNWTQSMKPLHYEDLTGMWECPDFFPVSITGSDGVETSSVGENGIKHVLKVSLIETLHDYYTIGSYDREKDVYVPDLGFVQNESAPRLDYGKYYASKTFYDDVKKRRILWGWVNESSPAKDDIEKGWSGLQSFPRKIWLDESGKELLQWPIEEIETLRGQQVNWQKKVLKAGSTLQVHGVTAAQADVEVSFKVKELEKADVIEPSWTDPQKICSQGDLSVMSGLGPFGLMVLASNDMEEYTSVYFRIFKSNDDTNKKTKYVVLMCSDQSRSSLNDENDKSTFGAFVAIDPSHQTISLRTLIDHSIVESYGGGGRTCITSRVYPKLAIGENANLFVFNKGTQSVDILTLSAWSLKSAQINGDLMSPFIEREESRSPNHQF

SEQ ID NO: 27 - protein, 654 aa, Artificial Sequence
  Synthetic
MRFPSIFTAVLFAASSALAAPVNTTTEDETAQIPAEAVIGYSDLEGDFDVAVLPFSNSTNNGLLFINTTIASIAAKEEGVSLEKREAEARSDPIKEHDYPWTNEMLTWQRSGFHFQPAKNFQSDPNAAMYYKGWYHFFYQYNPTGTAWDYTISWGHAVSRDLIHWLHLPMAMVPDHWYDAKGVWSGYSTLLPDGRVIVLYTGGTPELVQVQNLAVPADASDPLLLKWKKSSVNPILVPPPGIGTSDFRDPFPIWYNETDSNWHVLIGSKDSNHHGIVLLYKTKDFFNFTLLPSLLHTSTQSVGMFECVDLYPVATGGPLSNRGLEMSVDLSNGGIKHVLKASMDEERHDYYAIGTFDLDSFKWTPDDPSIDVGVGLRYDWGKFYASKTFFDTEKQRRILWGYVGEVDSKDDDKMKGWATLQNIPRTILLDTKTQSNLIIWPVEEVEDLRTDGNIFNDIKIGAGSSVQLDIGAASQLDIEAEFELDNSALDGAIEADVTYNCSTSGGAANRGLLGPFGLLVLANQDLTEQTATYFYVSRGTDGDLRTHFCQDELRSSKAGDIVKRVVGSVVPVLHGETWSLRILVDHSIIESFAQRGRAVATSRVYPTEAIYNKARLFLFNNATDAKVTAKSVKIWHMNSTHNHPFPGLESLFES

SEQ ID NO: 28 - protein, 565 aa, Artificial Sequence
  Synthetic
RSDPIKEHDYPWTNEMLTWQRSGFHFQPAKNFQSDPNAAMYYKGWYHFFYQYNPTGTAWDYTISWGHAVSRDLIHWLHLPMAMVPDHWYDAKGVWSGYSTLLPDGRVIVLYTGGTPELVQVQNLAVPADASDPLLLKWKKSSVNPILVPPPGIGTSDFRDPFPIWYNETDSNWHVLIGSKDSNHHGIVLLYKTKDFFNFTLLPSLLHTSTQSVGMFECVDLYPVATGGPLSNRGLEMSVDLSNGGIKHVLKASMDEERHDYYAIGTFDLDSFKWTPDDPSIDVGVGLRYDWGKFYASKTFFDTEKQRRILWGYVGEVDSKDDDKMKGWATLQNIPRTILLDTKTQSNLIIWPVEEVEDLRTDGNIFNDIKIGAGSSVQLDIGAASQLDIEAEFELDNSALDGAIEADVTYNCSTSGGAANRGLLGPFGLLVLANQDLTEQTATYFYVSRGTDGDLRTHFCQDELRSSKAGDIVKRVVGSVVPVLHGETWSLRILVDHSIIESFAQRGRAVATSRVYPTEAIYNKARLFLFNNATDAKVTAKSVKIWHMNSTHNHPFPGLESLFES

SEQ ID NO: 29 - DNA, 1947 bp, Artificial Sequence
  Synthetic
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgacttgaatcaaccttatagaaccggttaccacttccagccattaaaaaactggatgaacggcccaatgatttacaagggaatctatcatctgttttaccaatacaacccatacggtgccgtgtgggatgtaaggattgtctggggtcacagtacttccgtcgatttggttaattggataagccaacccccggcattcaacccatcacaaccatctgacatcaacggttgttggtcgggttctgttacgattctacctaatgggaagccagttatcctttatacaggtattgatcaaaacaagggtcaagttcagaatgtcgcggttccagtcaatatctctgacccatatttgcgtgaatggtccaaaccacctcaaaacccattgatgactaccaacgctgttaacggtatcaaccctgatagatttagagatccaactacagcttggctaggaagagatggtgagtggagagtcattgtgggttcatctaccgacgaccgccggggtttggccatattatacaagtcccgcgatttctttaattggactcaatctatgaaaccgttgcattacgaagatttgaccggaatgtgggaatgcccagacttcttcccagtttcaattacggggagtgatggtgtggaaacttcttccgtaggtgaaaacggtataaagcacgttctcaaggtcagcttaatcgaaactttgcatgactactataccattggttcgtatgacagagagaaggatgtctacgttcctgacttaggtttcgtccaaaatgaatccgctccacgtttggattacgggaaatactacgcctctaagacattttatgacgacgtcaaaaagcggagaattttatggggttgggttaacgaatcttcgccagctaaggacgatattgaaaagggctggtctggtttgcagtcatttccaagaaagatttggttggacgagagcggtaaagaattgctgcaatggccaatcgaagaaatagaaactctacgtggccaacaagttaactggcaaaagaaggttttgaaggctggttctaccttacaagtccacggtgttactgctgctcaagcggatgtagaggtttccttcaaagtcaaggaattggaaaaagcagacgtcatcgaaccctcctggaccgatccccaaaaaatatgttcgcagggtgacttgtctgttatgtctggtttaggtccgttcggtcttatggttcttgcttctaatgatatggaagaatacacttccgtttacttcagaatcttcaagagtaacgatgatactaataaaaagaccaagtatgttgtgctcatgtgttccgatcaatcaagaagttctttgaacgatgagaacgataagtcaacctttggggcctttgttgctattgatccatctcatcagaccatctctctccgaacattgattgaccactccatagtcgaatcatacggtggtggtggcagaacttgtatcacgagtagagtatatccaaagttggccatcggtgaaaatgcaaatttattcgtctttaacaagggtactcaatctgttgacattctgactttaagcgcttggtcccttaagagtgctcaaattaacggagacttgatgtctcctttcatcgagagagaagaaagtagatcacccaaccatcaattctaa

SEQ ID NO: 30 - DNA, 1965 bp, Artificial Sequence
  Synthetic
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctagatcagatcctattaaagagcatgactatccatggactaatgaaatgttgacatggcaacgtagtggatttcacttccagcccgctaagaacttccaatccgacccaaacgcagccatgtactacaagggctggtatcacttcttttaccaatacaatccgaccggtactgcttgggattacacgatctcttggggtcatgctgtctcgcgggacttaatacactggcttcatctgccaatggctatggtaccagatcactggtatgatgcgaagggtgtgtggtccggttactctaccctattgccagatggtagagttattgtcttatatactggtggtaccccagaattggttcaagttcaaaacttggccgttcctgctgacgcctctgatccactgttgttgaaatggaagaagtcctcagtcaaccccatccttgttccgccaccagggattggaactagcgacttcagggatccatttcctatctggtacaatgaaacagactccaactggcacgtcttgataggttctaaagactccaaccaccatggtattgtattattgtataagactaaggacttctttaacttcacattgcttccatctttattgcacaccagtacccagagcgttggtatgttcgaatgcgtggatctctacccagtcgctactggtgggccactatctaatagaggtttggaaatgagcgttgatctctcaaatggtggtatcaaacatgttttgaaggcttctatggatgaggaaagacatgactactatgcgattggcacctttgacttagattctttcaaatggacgcccgacgatccaagtatcgacgttggtgtcggtctaagatacgattggggtaagttctacgcttctaagaccttttttgatactgaaaagcaacgccgaattttatggggctatgtcggtgaagttgactccaaggatgatgacaagatgaaaggttgggcaaccttacaaaatatacctagaactatcttgcttgacacgaaaactcaatctaacttgattatctggccagtcgaggaagttgaagatttgagaactgacggcaacattttcaacgatataaaaattggtgctggttcttcagtacaattggatattggtgccgcttcgcagttggacatcgaagccgaatttgaactagataacagtgctttggacggcgctattgaagctgatgtcacttacaattgttcaacttcgggtggtgccgcaaatagaggtttgctggggcctttcggtttacttgttttagctaaccaagacttgacagaacaaaccgctacatacttctacgtgtccagaggtaccgatggtgatttgagaacccacttctgtcaagacgaattacgttcctccaaggcaggagacattgtcaagcgcgttgttggttctgtggtgccagttctacatggtgaaacttggtccttgagaattttggttgaccactctatcatcgaaagctttgcacaaagaggacgggctgttgctacctctagggtctacccaactgaggcaatctacaacaaagccagactgtttttgttcaacaatgctacagacgctaaggttactgccaagagtgttaaaatatggcatatgaactctacacacaaccatccattccctggtttagaatcgctattcgaatcataa

SEQ ID NO: 31 - protein, 542 aa, Artificial Sequence
  Synthetic
SSVQPSAAERLTWERTAFHFQPAKNFIYDPNGPLFHMGWHHLFYQYNPYAPVWGNMSWGHAVSKDMINWFELPVALVPTEWYDIEGVLSGSTTALPNGQIFALYTGNANDFSQLQCKAVPVDVSDPLLVKWVKYDGNPILYTPPGIGLKDYRDPSTVWTGPDGKHRMIMGTKRGTTGLVLVYHTTDFTNYVMLDEPLHSVPNTDMWECVDLFPVSTTNDSALDIAAYGSGIKHVLKESWEGHAMDFYSIGTYDAINDKWTPDNPELDVGIGLRCDYGRFFASKSLYDPLKKRRVTWGYVAESDSADQDVSRGWATIYNVARTIVLDRKTGTHLLQWPVEELESLRSNVREFKEMTLEPGSIVPLDIGSATQLDIIATFEVDQEALKATSDANDEYACTTSSGAAERGSFGPFGIAVLADGTLSELTPVYFYIAKNTKGGVDTHFCTDKLRSSLDYDSEKVVYGSTIPVLDGEQITMRVLVDHSVVEGFAQGGRTVITSRVYPTKAIYEGAKLFVFNNATTTNVKATLNVWQMSHALIQPYPF

SEQ ID NO: 32 - protein, 631 aa, Artificial Sequence
  Synthetic
MRFPSIFTAVLFAASSALAAPVNTTTEDETAQIPAEAVIGYSDLEGDFDVAVLPFSNSTNNGLLFINTTIASIAAKEEGVSLEKREAEASSVKHSQSDRLRWERTAYHFQPAKNFIYDPNGPLFHMGWYHLFYQYNPYAPIWGNMSWGHAVSKDMIHWFELPVAIVPTEWYDIEGVLSGSTTALPNGQIFALYTGNAKDFSQLQCKAVPLNASDPLLVEWVKYEDNPILYIPPGIGPKDYRDPSTVWTGPDGKHRMIMGTKQNGTGMVHVYHTTDFINYVLLDEPLHSVPNTDMWECVDFYPVSTINDSALDIAAYGSDIKHVIKESWEGHGMDLYSIGTYDAYKDKWTPDNPEFDVGIGLRVDYGRFFASKSLYDPLKKRRVTWGYVAESDSSDQDLNRGWATIYNVGRTVVLDRKTGTHLLHWPVEEIESLRSNVREFNEIELVPGSIIPLDIGMATQLDIVATFKVDPEALMAKSDINSEYGCTTSSGATQRGSLGPFGIVVLADVALSELTPVYFYIAKNIDGGLVTHFCTDKLRSSLDYDGERVVYGSTVPVLDGEELTMRLLVDHSVVEGFAQGGRTVMTSRVYPTNAIYEEAKIFLFNNATGASVKASLKIWQMGSASIQAYPF

SEQ ID NO: 33 - protein, 542 aa, Artificial Sequence
  Synthetic
SSVKHSQSDRLRWERTAYHFQPAKNFIYDPNGPLFHMGWYHLFYQYNPYAPIWGNMSWGHAVSKDMIHWFELPVAIVPTEWYDIEGVLSGSTTALPNGQIFALYTGNAKDFSQLQCKAVPLNASDPLLVEWVKYEDNPILYIPPGIGPKDYRDPSTVWTGPDGKHRMIMGTKQNGTGMVHVYHTTDFINYVLLDEPLHSVPNTDMWECVDFYPVSTINDSALDIAAYGSDIKHVIKESWEGHGMDLYSIGTYDAYKDKWTPDNPEFDVGIGLRVDYGRFFASKSLYDPLKKRRVTWGYVAESDSSDQDLNRGWATIYNVGRTVVLDRKTGTHLLHWPVEEIESLRSNVREFNEIELVPGSIIPLDIGMATQLDIVATFKVDPEALMAKSDINSEYGCTTSSGATQRGSLGPFGIVVLADVALSELTPVYFYIAKNIDGGLVTHFCTDKLRSSLDYDGERVVYGSTVPVLDGEELTMRLLVDHSVVEGFAQGGRTVMTSRVYPTNAIYEEAKIFLFNNATGASVKASLKIWQMGSASIQAYPF

SEQ ID NO: 34 - protein, 631 aa, Artificial Sequence
  Synthetic
MRFPSIFTAVLFAASSALAAPVNTTTEDETAQIPAEAVIGYSDLEGDFDVAVLPFSNSTNNGLLFINTTIASIAAKEEGVSLEKREAEASSVKHSQSDRLRWERTAYHFQPAKNFIYDPNGPLFHMGWYHLFYQYNPYAPIWGNMSWGHAVSKDMIHWFELPVAMVPTEWYDIEGVLSGSTTALPNGQIFALYTGNAKDFSQLQCKAVPLNASDPLLVDWVKYEDNPILYIPPGIGPKDYRDPSTVWTGPDGKHRMIMGTKQNGTGMVHVYHTTDFINYVLLDEPLHSVPNTDMWECVDFYPVSTINDSALDIAAYGSDIKHVIKESWEGHGMDLYSIGTYDAYKDKWTPDNPELDVGIGLRVDYGRLFASKSLYDPLKKRRVTWGYVGESDSPDQDINRGWATIYNVGRTVVLDRKTGTHLLHWPVEEIESLRSNVREFNEIELVPGSIIPLDIGMATQLDIVATFKVDPEALMAKSDINSEYGCTTSSGATQRGSLGPFGIVVLADLALSELTPLYFYIAKNTDGGLVTHFCTDKLRSSLDYDGERVVYGGTVPVLDGEELTMRLLVDHSVVEGFAQGGRTVITSRVYPTNAIYEEAKIFLFNNATGASVKASLKIWQMGSASIQAYPF

SEQ ID NO: 35 - protein, 542 aa, Artificial Sequence
  Synthetic
SSVKHSQSDRLRWERTAYHFQPAKNFIYDPNGPLFHMGWYHLFYQYNPYAPIWGNMSWGHAVSKDMIHWFELPVAMVPTEWYDIEGVLSGSTTALPNGQIFALYTGNAKDFSQLQCKAVPLNASDPLLVDWVKYEDNPILYIPPGIGPKDYRDPSTVWTGPDGKHRMIMGTKQNGTGMVHVYHTTDFINYVLLDEPLHSVPNTDMWECVDFYPVSTINDSALDIAAYGSDIKHVIKESWEGHGMDLYSIGTYDAYKDKWTPDNPELDVGIGLRVDYGRLFASKSLYDPLKKRRVTWGYVGESDSPDQDINRGWATIYNVGRTVVLDRKTGTHLLHWPVEEIESLRSNVREFNEIELVPGSIIPLDIGMATQLDIVATFKVDPEALMAKSDINSEYGCTTSSGATQRGSLGPFGIVVLADLALSELTPLYFYIAKNTDGGLVTHFCTDKLRSSLDYDGERVVYGGTVPVLDGEELTMRLLVDHSVVEGFAQGGRTVITSRVYPTNAIYEEAKIFLFNNATGASVKASLKIWQMGSASIQAYPF

SEQ ID NO: 36 - DNA, 1896 bp, Artificial Sequence
  Synthetic
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctagttccgttaaacattctcagtcagatcgattgaggtgggaacgtactgcctaccactttcaaccagcaaagaacttcatatatgaccctaatggtccacttttccacatgggatggtaccatctattttaccaatataacccgtatgctccaatttggggcaatatgtcttggggtcacgctgtgtccaaggacatgatccattggttcgagctgcccgtcgctatcgttccaacggaatggtacgatattgaaggtgtattaagcggttcgacaactgcgttgccaaacggtcaaattttcgccttgtacaccggtaatgctaaggatttttctcaattacaatgcaaagctgtccctttgaacgcttccgacccattgttggttgaatgggttaagtacgaagataaccctatcctatatattccaccaggcatcggtcctaaggactacagagatccatctaccgtgtggacaggtccagatggtaaacacagaatgattatgggaaccaagcaaaacggtactgggatggttcatgtctaccacaccactgactttataaattatgtcttattagacgagccgttgcactccgtcccaaacaccgatatgtgggaatgtgtggacttctacccagtatctactatcaatgacagcgcgttggatattgcagcctacggttcagacatcaagcatgttataaaagaatcttgggaaggtcatggtatggatttatactctattggtacttatgacgcttacaaggataagtggacgccagataaccccgagttcgatgttgggattggtctgagagttgattacggcagattctttgcttccaagagcttgtacgacccgttgaagaagagaagagtcacatggggttatgttgctgaaagtgattcttccgaccaagacctcaatagaggttgggccacaatctataacgttggtagaactgtcgtcttggaccggaaaaccggtacacacctattacattggccagtggaggaaattgaatctctgcgttcgaacgtcagagaatttaatgaaattgaattggttccaggatcgatcataccattggatattggtatggctactcaattggacatcgttgccaccttcaaagtagacccagaagctcttatggctaagtccgatattaactctgaatacggttgtaccacttcctcaggtgctactcagcgtgggtctttaggcccttttggtatcgttgttttggctgacgtagctctatcggagttaaccccagtttacttctatatcgcaaagaatatcgatggtggtctggtcactcacttctgtaccgataaattgcgctctagtttggactacgatggagaaagagttgtttacggttcaactgttccagtcttggacggtgaagaattaaccatgagattgctggtggatcatagtgtagtcgaaggtttcgctcaaggtggtagaactgttatgacctccagagtctaccccactaacgccatctatgaagaggcgaagatttttcttttcaataacgcgactggcgctagtgttaaagcatctttgaagatttggcaaatgggttctgcctctattcaggcttatcccttctaa

SEQ ID NO: 37 - DNA, 1896 bp, Artificial Sequence
  Synthetic
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctagttccgttaaacattctcagtcagatcgattgaggtgggaacgtactgcctaccactttcaaccagcaaagaacttcatatatgaccctaatggtccacttttccacatgggatggtaccatctattttaccaatataacccgtatgctccaatttggggcaatatgtcttggggtcacgctgtgtccaaggacatgatccattggttcgagctgcccgtcgctatggttccaacggaatggtacgatattgaaggtgtcttgtctgggagcaccacagctttgcctaacggtcaaatcttcgccttatacactggtaatgcgaaagatttttcccaattacaatgcaaggctgttccattgaacgcctcggacccattgctcgtagattgggtcaagtacgaagataacccaattttgtatatccccccaggtattggaccaaaggactacagagatccgagtaccgtgtggactggtcctgacggtaaacacagaatgatcatgggtaccaagcaaaacggcactggtatggttcacgtataccatacaaccgactttattaattatgttttattggacgaaccattgcactctgttccaaatactgatatgtgggagtgtgtcgatttctacccagtctctacgataaacgacagcgcactcgatatagctgcttatggtagtgatattaagcacgttattaaagaatcttgggaaggtcatggtatggacttgtactccatcggtacttacgatgcttacaaggataagtggaccccagacaaccctgaattagacgttggtatcgggctaagagtggactatggtagattgttcgcatcgaaaagcctttacgatccactgaagaaaagaagagtcacttggggttacgttggcgagtctgattctccagatcaggacattaacagaggttgggcgaccatctataatgttggacgtaccgtcgttttggatagaaagactggtactcatctactgcactggcctgtcgaagaaatcgaatcattaagaagtaatgttagagaatttaacgaaattgagttggtaccaggttctataattcctttggacattggtatggccacacaattggacatcgttgctacattcaaggttgatccagaagctttaatggctaagtctgacataaactccgaatacggttgtaccacttcctccggtgcgactcaaagaggttcgttgggtccattcggtatcgtcgttctagccgatttggctctctctgaattgactccattatacttttatatcgctaagaacaccgatgggggcttggtaacacacttctgtactgataaattaagatcaagtttggattacgacggtgaacgcgtcgtatacggtggtacggttcccgtgttagacggggaagaactcaccatgaggctattggtcgatcattctgttgttgagggttttgctcaaggtggaagaaccgttattactagccgtgtctatcccacaaatgctatttatgaagaagccaagattttcctttttaacaacgctaccggtgcatccgttaaggcttctttgaagatatggcaaatgggtagcgcttctatccaagcctacccattctaa

SEQ ID NO: 38 - protein, 549 aa, Artificial Sequence
  Synthetic
VPGKLESNADVEWQRSAYHFQPDKNFISDPDGPMYHMGWYHLFYQYNPESAIWGNITWGHSVSRDMINWFHLPFAMVPDHWYDIEGVMTGSATVLPNGQIIMLYTGNAYDLSQLQCLAYAVNSSDPLLLEWKKYEGNPILFPPPGVGYKDFRDPSTLWMGPDGEWRMVMGSKHNETIGCALVYRTTNFTHFELNEEVLHAVPHTGMWECVDLYPVSTTHTNGLEMKDNGPNVKYILKQSGDEDRHDWYAIGTFDPEKDKWYPDDPENDVGIGLRYDYGKFYASKTFYDQHKKRRVLWGYVGETDPPKSDLLKGWANILNIPRSVVLDTQTETNLIQWPIEEVEKLRSKKYDEFKDVELRPGSLIPLEIGTATQLDISATFEIDEKKLESTLEADVLFNCTTSEGSVGRGVLGPFGIVVLADANRSEQLPVYFYIAKDTDGTSRTYFCADESRSSKDKDVGKWVYGSSVPVLEGENYNMRLLVDHSIVEGFAQGGRTVVTSRVYPTMAIYGAAKIFLFNNATGISVKASLKIWKMAEAQLDPFPLSGWSS

SEQ ID NO: 39 - protein, 644 aa, Artificial Sequence
  Synthetic
MRFPSIFTAVLFAASSALAAPVNTTTEDETAQIPAEAVIGYSDLEGDFDVAVLPFSNSTNNGLLFINTTIASIAAKEEGVSLEKREAEADEEAAGGFPWSNEMLQWQRSGYHFQTAKNYMSDPNGLMYYRGWYHMFFQYNPVGTDWDDGMEWGHAVSRNLVQWRTLPIAMVADQWYDILGVLSGSMTVLPNGTVIMIYTGATNASAVEVQCIATPADPNDPLLRRWTKHPANPVIWSPPGVGTKDFRDSMTAWYDESDDTWRTLLGSKDDNNGHHDGIAMMYKTKDFLNYELIPGILHRVERTGEWECIDFYPVGHRTSDNSSEMLHVLKASMDDERHDYYSLGTYDSAANRWTPIDPELDLGIGLRYDWGKFYASTSFYDPAKKRRVLMGYVGEVDSKRADVVKGWASIQSVPRTIALDEKTRTNLLLWPVEEIETLRLNATQLSDVTLNTGSVIHIPLRQGTQLDIEATFHLDASAVAALNEADVGYNCSSSGGAVNRGALGPFGLLVLAAGDRRGEQTAVYFYVSRGLDGGLHTSFCQDELRSSRAKDVTKRVIGSTVPVLDGEAFSMRVLVDHSIVQGFAMGGRTTMTSRVYPMEAYQEAKVYLFNNATGASVTAERLVVHDMDSAHNQLSNMDDYSYVQ

SEQ ID NO: 40 - protein, 555 aa, Artificial Sequence
  Synthetic
DEEAAGGFPWSNEMLQWQRSGYHFQTAKNYMSDPNGLMYYRGWYHMFFQYNPVGTDWDDGMEWGHAVSRNLVQWRTLPIAMVADQWYDILGVLSGSMTVLPNGTVIMIYTGATNASAVEVQCIATPADPNDPLLRRWTKHPANPVIWSPPGVGTKDFRDSMTAWYDESDDTWRTLLGSKDDNNGHHDGIAMMYKTKDFLNYELIPGILHRVERTGEWECIDFYPVGHRTSDNSSEMLHVLKASMDDERHDYYSLGTYDSAANRWTPIDPELDLGIGLRYDWGKFYASTSFYDPAKKRRVLMGYVGEVDSKRADVVKGWASIQSVPRTIALDEKTRTNLLLWPVEEIETLRLNATQLSDVTLNTGSVIHIPLRQGTQLDIEATFHLDASAVAALNEADVGYNCSSSGGAVNRGALGPFGLLVLAAGDRRGEQTAVYFYVSRGLDGGLHTSFCQDELRSSRAKDVTKRVIGSTVPVLDGEAFSMRVLVDHSIVQGFAMGGRTTMTSRVYPMEAYQEAKVYLFNNATGASVTAERLVVHDMDSAHNQLSNMDDYSYVQ

SEQ ID NO: 41 - protein, 644 aa, Artificial Sequence
  Synthetic
MRFPSIFTAVLFAASSALAAPVNTTTEDETAQIPAEAVIGYSDLEGDFDVAVLPFSNSTNNGLLFINTTIASIAAKEEGVSLEKREAEADEEAAGGFPWSNEMLQWQRSGYHFQTAKNYMSDPNGLMYYRGWNHMFFQYNPVGTDWDDGMEWGHAVSRNLVQWRTLPIAMVADQWYDILGVLSGSMTVLPNGTVIMIYTGATNASAVEVQCIATPADPTDPLLRRWTKHPANPVIWSPPGVGTKDFRDPMTAWYDESDDTWRTLLGSKDDNNGHHDGIAMMYKTKDFLNYELIPGILHRVERTGEWECIDFYPVGRRTSDNSSEMLHVLKASMDDERHDYYSLGTYDSAANRWTPIDPELDLGIGLRYDWGKFYASTSFYDPAKKRRVLMGYVGEVDSKRADVVKGWASIQSVPRTIALDEKTRTNLLLWPVEEIETLRLNATELSDVTLNTGSVIHIPLRQGTQLDIEATFHLDASAVAAFNEADVGYNCSSSGGAVNRGALGPFGLLVLAAGDRRGEQTAVYFYVSRGLDGGLHTSFCQDELRSSRAKDVTKRVIGSTVPVLDGEAFSMRVLVDHSIVQGFAMGGRTTMTSRVYPMEAYQEAKVYLFNNATGASVTAERLVVHEMDSAHNQLSNMDDHSYVQ

SEQ ID NO: 42 - protein, 555 aa, Artificial Sequence
  Synthetic
DEEAAGGFPWSNEMLQWQRSGYHFQTAKNYMSDPNGLMYYRGWNHMFFQYNPVGTDWDDGMEWGHAVSRNLVQWRTLPIAMVADQWYDILGVLSGSMTVLPNGTVIMIYTGATNASAVEVQCIATPADPTDPLLRRWTKHPANPVIWSPPGVGTKDFRDPMTAWYDESDDTWRTLLGSKDDNNGHHDGIAMMYKTKDFLNYELIPGILHRVERTGEWECIDFYPVGRRTSDNSSEMLHVLKASMDDERHDYYSLGTYDSAANRWTPIDPELDLGIGLRYDWGKFYASTSFYDPAKKRRVLMGYVGEVDSKRADVVKGWASIQSVPRTIALDEKTRTNLLLWPVEEIETLRLNATELSDVTLNTGSVIHIPLRQGTQLDIEATFHLDASAVAAFNEADVGYNCSSSGGAVNRGALGPFGLLVLAAGDRRGEQTAVYFYVSRGLDGGLHTSFCQDELRSSRAKDVTKRVIGSTVPVLDGEAFSMRVLVDHSIVQGFAMGGRTTMTSRVYPMEAYQEAKVYLFNNATGASVTAERLVVHEMDSAHNQLSNMDDHSYVQ

SEQ ID NO: 43 - protein, 649 aa, Artificial Sequence
  Synthetic
MRFPSIFTAVLFAASSALAAPVNTTTEDETAQIPAEAVIGYSDLEGDFDVAVLPFSNSTNNGLLFINTTIASIAAKEEGVSLEKREAEASGPYSASGGFPWSNAMLQWQRTGYHFQPEKNYQNDPNGPVYYKGWYHFFYQHNPGGTGWGNISWGHAVSRDMVHWRHLPLAMVPEHWYDIEGVLTGSITVLPDGRVILLYTGNTETFAQVTCLAEAADPSDPLLREWAKHPANPVVYPPPGIGMKDYRDPTTAWFDNSDNTWRIIIGSKNDTDHSGIVFTYKTKDFVSYELIPGYLYRGPAGTGMYECIDLFAVGGGRAASDMYNSTAEDVLYVLKESSDDDRRDYYALGRFDAAANTWTPIDTERELGVALRYDYGRYDTSKSFYDPVKQRRIVWGYVVETDSWSADAAKGWANLQSIPRTVELDEKTRTNLVQWPVGELNTLRINTTDLSDITVGAGSVDSLPLHQTSQLDIEASFRINASTIEALNEVDVGYNCTMTSGAATRGALGPFGILVLANVALTEQTAVYFYVSKGLDGGLRTHFCHDELRSTHATDVAKEVVGSTVPVLDGEDFSVRVLVDHSIVQSFVMGGRMTATSRAYPTEAIYAAAGVYLFNNATGASITAEKLVVHDMDSSYNRIFTDEDLLVLD

SEQ ID NO: 44 - protein, 560 aa, Artificial Sequence
  Synthetic
SGPYSASGGFPWSNAMLQWQRTGYHFQPEKNYQNDPNGPVYYKGWYHFFYQHNPGGTGWGNISWGHAVSRDMVHWRHLPLAMVPEHWYDIEGVLTGSITVLPDGRVILLYTGNTETFAQVTCLAEAADPSDPLLREWAKHPANPVVYPPPGIGMKDYRDPTTAWFDNSDNTWRIIIGSKNDTDHSGIVFTYKTKDFVSYELIPGYLYRGPAGTGMYECIDLFAVGGGRAASDMYNSTAEDVLYVLKESSDDDRRDYYALGRFDAAANTWTPIDTERELGVALRYDYGRYDTSKSFYDPVKQRRIVWGYVVETDSWSADAAKGWANLQSIPRTVELDEKTRTNLVQWPVGELNTLRINTTDLSDITVGAGSVDSLPLHQTSQLDIEASFRINASTIEALNEVDVGYNCTMTSGAATRGALGPFGILVLANVALTEQTAVYFYVSKGLDGGLRTHFCHDELRSTHATDVAKEVVGSTVPVLDGEDFSVRVLVDHSIVQSFVMGGRMTATSRAYPTEAIYAAAGVYLFNNATGASITAEKLVVHDMDSSYNRIFTDEDLLVLD

SEQ ID NO: 45 - protein, 644 aa, Artificial Sequence
  Synthetic
MRFPSIFTAVLFAASSALAAPVNTTTEDETAQIPAEAVIGYSDLEGDFDVAVLPFSNSTNNGLLFINTTIASIAAKEEGVSLEKREAEAANAFPWSNAMLQWQRTGFHFQPDKYYQNDPNGPVYYGGWYHFFYQYNPSGSVWEPQIVWGHAVSKDLIHWRHLPPALVPDQWYDIKGVLTGSITVLPDGKVILLYTGNTETFAQVTCLAEPADPSDPLLREWVKHPANPVVFPPPGIGMKDFRDPTTAWYDESDGTWRTIIGSKNDSDHSGIVFSYKTKDFISYELMPGYMYRGPKGTGEYECIDLYAVGGGRKASDMYNSTAEDVLYVLKESSDDDRHDWYSLGRFDAAANKWTPIDTELELGVGLRYDWGKYYASKSFYDPVKKRRVVWAYVGETDSERADITKGWANLQSIPRTVELDEKTRTNLIQWPVEELNTLRINTTDLSGITVGAGSVAFLPLHQTAQLDIEATFRIDASAIEALNEADVSYNCTTSRGAATRGALGPFGLLVLANHALTEQTGVYFYVSKGLDGGLRTHFCHDELRSSHASDVVKRVVGSTVPVLDGEDFSVRVLVDHSIVQSFAMGGRLTATSRAYPTEAIYAAAGVYMFNNATGTSVTAEKLVVHDMDSSYNHIYTDGDLVVVD

SEQ ID NO: 46 - protein, 555 aa, Artificial Sequence
  Synthetic
ANAFPWSNAMLQWQRTGFHFQPDKYYQNDPNGPVYYGGWYHFFYQYNPSGSVWEPQIVWGHAVSKDLIHWRHLPPALVPDQWYDIKGVLTGSITVLPDGKVILLYTGNTETFAQVTCLAEPADPSDPLLREWVKHPANPVVFPPPGIGMKDFRDPTTAWYDESDGTWRTIIGSKNDSDHSGIVFSYKTKDFISYELMPGYMYRGPKGTGEYECIDLYAVGGGRKASDMYNSTAEDVLYVLKESSDDDRHDWYSLGRFDAAANKWTPIDTELELGVGLRYDWGKYYASKSFYDPVKKRRVVWAYVGETDSERADITKGWANLQSIPRTVELDEKTRTNLIQWPVEELNTLRINTTDLSGITVGAGSVAFLPLHQTAQLDIEATFRIDASAIEALNEADVSYNCTTSRGAATRGALGPFGLLVLANHALTEQTGVYFYVSKGLDGGLRTHFCHDELRSSHASDVVKRVVGSTVPVLDGEDFSVRVLVDHSIVQSFAMGGRLTATSRAYPTEAIYAAAGVYMFNNATGTSVTAEKLVVHDMDSSYNHIYTDGDLVVVD

SEQ ID NO: 47 - protein, 650 aa, Artificial Sequence
  Synthetic
MRFPSIFTAVLFAASSALAAPVNTTTEDETAQIPAEAVIGYSDLEGDFDVAVLPFSNSTNNGLLFINTTIASIAAKEEGVSLEKREAEADDPPSDSEDYPWTNEMLKWQRTGYHFQPPNHFMADPNAAMYYKGWYHFFYQYNPNGSAWDYSISWGHAVSKDMIHWLHLPVAMVPDHWYDSKGVWSGYATTLPDGRIIVLYTGGTDQLVQVQNLAEPADPSDPLLIEWKKSNGNPILMPPPGVGPHDFRDPFPVWYNESDSTWHMLIGSKDDNHYGTVLIYTTKDFETYTLLPDILHKTKDSVGMLECVDLYPVATTGNQIGNGLEMKGGSGKGIKHVLKASMDDERHDYYAIGTFDLESFSWVPDDDTIDVGVGLRYDYGKFYASKTFYDQEKKRRILWGYVGEVDSKADDILKGWASVQNIARTILFDAKTRSNLLVWPVEELDALRTSGKEFNGVVVEPGSTYHLDVGTATQLDIEAEFEINKEAVDAVVEADVTYNCSTSDGAAHRGLLGPFGLLVLANEKMTEKTATYFYVSRNVDGGLQTHFCQDELRSSKANDITKRVVGHTVPVLHGETFSLRILVDHSIVESFAQKGRAVATSRVYPTEAIYDSTRVFLFNNATSATVTAKSVKIWHMNSTHNHPFPGFPAP

SEQ ID NO: 48 - protein, 561 aa, Artificial Sequence
  Synthetic
DDPPSDSEDYPWTNEMLKWQRTGYHFQPPNHFMADPNAAMYYKGWYHFFYQYNPNGSAWDYSISWGHAVSKDMIHWLHLPVAMVPDHWYDSKGVWSGYATTLPDGRIIVLYTGGTDQLVQVQNLAEPADPSDPLLIEWKKSNGNPILMPPPGVGPHDFRDPFPVWYNESDSTWHMLIGSKDDNHYGTVLIYTTKDFETYTLLPDILHKTKDSVGMLECVDLYPVATTGNQIGNGLEMKGGSGKGIKHVLKASMDDERHDYYAIGTFDLESFSWVPDDDTIDVGVGLRYDYGKFYASKTFYDQEKKRRILWGYVGEVDSKADDILKGWASVQNIARTILFDAKTRSNLLVWPVEELDALRTSGKEFNGVVVEPGSTYHLDVGTATQLDIEAEFEINKEAVDAVVEADVTYNCSTSDGAAHRGLLGPFGLLVLANEKMTEKTATYFYVSRNVDGGLQTHFCQDELRSSKANDITKRVVGHTVPVLHGETFSLRILVDHSIVESFAQKGRAVATSRVYPTEAIYDSTRVFLFNNATSATVTAKSVKIWHMNSTHNHPFPGFPAP

SEQ ID NO: 49 - protein, 651 aa, Artificial Sequence
  Synthetic
MRFPSIFTAVLFAASSALAAPVNTTTEDETAQIPAEAVIGYSDLEGDFDVAVLPFSNSTNNGLLFINTTIASIAAKEEGVSLEKREAEANLMRLRENDYPWTNDMLRWQRTGFHFQPEKNFQADPNAAMFYKGWYHFFYQYNPTGVAWDYTISWGHAVSKDLLHWNYLPMALRPDHWYDRKGVWSGYSTLLPDGRIVVLYTGGTKELVQVQNLAVPVNLSDPLLLEWKKSHVNPILVPPPGIEDHDFRDPFPVWYNESDSRWHVVIGSKDPEHYGIVLIYTTKDFVNFTLLPNILHSTKQPVGMLECVDLFPVATTDSRANQALDMTTMRPGPGLKYVLKASMDDERHDYYALGSFDLDSFTFTPDDETIDVGVGLRYDWGKFYASKTFYDQEKHRRVLWGYVGEVDSKRDDALKGWASLQNIPRTILFDTKTKSNLILWPVEEVESLRTINKNFNSIPLYPGSTYQLDVGEATQLDIVAEFEVDEKAIEATAEADVTYNCSTSGGAANRGVLGPFGLLVLANQELSEQTATYFYVSRGIDGNLRTHFCQDELRSSKAGAITKRVVGSTVPVLHGETWALRILVDHSIVESFAQRGRAVATSRVYPTEAIYSSARVFLFNNATDAIVTAKTVNVWHMNSTYNHVFPGLVAP

SEQ ID NO: 50 - protein, 562 aa, Artificial Sequence
  Synthetic
NLMRLRENDYPWTNDMLRWQRTGFHFQPEKNFQADPNAAMFYKGWYHFFYQYNPTGVAWDYTISWGHAVSKDLLHWNYLPMALRPDHWYDRKGVWSGYSTLLPDGRIVVLYTGGTKELVQVQNLAVPVNLSDPLLLEWKKSHVNPILVPPPGIEDHDFRDPFPVWYNESDSRWHVVIGSKDPEHYGIVLIYTTKDFVNFTLLPNILHSTKQPVGMLECVDLFPVATTDSRANQALDMTTMRPGPGLKYVLKASMDDERHDYYALGSFDLDSFTFTPDDETIDVGVGLRYDWGKFYASKTFYDQEKHRRVLWGYVGEVDSKRDDALKGWASLQNIPRTILFDTKTKSNLILWPVEEVESLRTINKNFNSIPLYPGSTYQLDVGEATQLDIVAEFEVDEKAIEATAEADVTYNCSTSGGAANRGVLGPFGLLVLANQELSEQTATYFYVSRGIDGNLRTHFCQDELRSSKAGAITKRVVGSTVPVLHGETWALRILVDHSIVESFAQRGRAVATSRVYPTEAIYSSARVFLFNNATDAIVTAKTVNVWHMNSTYNHVFPGLVAP

SEQ ID NO: 51 - protein, 650 aa, Artificial Sequence
  Synthetic
MRFPSIFTAVLFAASSALAAPVNTTTEDETAQIPAEAVIGYSDLEGDFDVAVLPFSNSTNNGLLFINTTIASIAAKEEGVSLEKREAEADDPPSDSEDYPWTNEMLKWQRTGYHFQPPNHFMADPNAAMYYKGWYHFFYQYNPNGSAWDYSISWGHAVSKDMIHWLHLPVAMVPDHWYDSKGVWSGYATTLPDGRIIVLYTGGTDQLVQVQNLAEPADPSDPLLIEWKKSNGNPILMPPPGVGPHDFRDPFPVWYNESDSTWHMLIGSKDDNHYGTVLIYTTKDFETYTLLPDILHKTKDSVGMLECVDLYPVATTGNQIGNGLEMKGGSGKGIKHVLKASMDDERHDYYAIGTFDLESFSWVPDDDTIDVGVGLRYDYGKFYASKTFYDQEKKRRILWGYVGEVDSKADDILKGWASVQNIARTILFDAKTRSNLLVWPVEELDALRTSGKEFNGVVVEPGSTYHLDVGTATQLDIEAEFEINKEAVDAVVEADVTYNCSTSDGAAHRGLLGPFGLLVLANEKMTEKTATYFYVSRNADGGLQTHFCQDELRSSKANDITKRVVGHTVPVLHGETFSLRILVDHSIVESFAQKGRAVATSRVYPTEAIYDSTRVFLFNNATSATVTAKSVKIWHMNSTHNHPFPGFPAP

SEQ ID NO: 52 - protein, 561 aa, Artificial Sequence
  Synthetic
DDPPSDSEDYPWTNEMLKWQRTGYHFQPPNHFMADPNAAMYYKGWYHFFYQYNPNGSAWDYSISWGHAVSKDMIHWLHLPVAMVPDHWYDSKGVWSGYATTLPDGRIIVLYTGGTDQLVQVQNLAEPADPSDPLLIEWKKSNGNPILMPPPGVGPHDFRDPFPVWYNESDSTWHMLIGSKDDNHYGTVLIYTTKDFETYTLLPDILHKTKDSVGMLECVDLYPVATTGNQIGNGLEMKGGSGKGIKHVLKASMDDERHDYYAIGTFDLESFSWVPDDDTIDVGVGLRYDYGKFYASKTFYDQEKKRRILWGYVGEVDSKADDILKGWASVQNIARTILFDAKTRSNLLVWPVEELDALRTSGKEFNGVVVEPGSTYHLDVGTATQLDIEAEFEINKEAVDAVVEADVTYNCSTSDGAAHRGLLGPFGLLVLANEKMTEKTATYFYVSRNADGGLQTHFCQDELRSSKANDITKRVVGHTVPVLHGETFSLRILVDHSIVESFAQKGRAVATSRVYPTEAIYDSTRVFLFNNATSATVTAKSVKIWHMNSTHNHPFPGFPAP

SEQ ID NO: 53 - DNA, 1935 bp, Artificial Sequence
  Synthetic
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgacgaagaggctgccggtggatttccctggtcaaacgaaatgttacaatggcagagatccggttaccacttccaaacagcaaaaaattatatgtctgatcctaacggcctaatgtactataggggttggtaccatatgttcttccaatacaacccagtcgggactgattgggacgacggtatggaatggggtcacgctgtgtcgcgtaatttggtacaatggagaacgttgccaatagctatggttgccgatcaatggtatgatattctgggtgttctttctggttctatgaccgtcttgccaaacggtactgttatcatgatctacaccggtgctactaatgcgagcgctgtcgaagttcaatgtattgcaaccccagccgatccgaacgaccctttgttaagaagatggactaagcatccagctaaccctgtgatctggagtccaccaggtgtagggacaaaggattttcgagactccatgaccgcttggtacgacgagtcagatgacacttggagaaccttgttgggctccaaggacgataacaatggtcaccatgatggtattgctatgatgtataaaactaaggatttcctaaattacgaacttatcccaggcatactgcaccgtgtcgaaaggacaggtgaatgggaatgcatcgacttttacccggttggtcatagaacgtctgataactctagcgaaatgttgcacgttttgaaagcctctatggatgacgaacggcacgattattactccttaggtacttacgatagtgctgccaacagatggaccccaattgaccccgaactagacttgggtattggattgagatatgattggggtaagttttacgctagcacttcattctacgatccagcaaagaaacgtcgagtcttaatgggatatgttggtgaggttgactccaagagagctgacgtcgtgaagggttgggcttctatccaatctgttccaagaacaattgcattggacgaaaagactagaaccaacctgctgttatggcccgttgaggaaatcgaaacattgagactaaatgctacccaactctcggatgtcaccttgaatactggttctgtcattcatattcctttgagacaaggtacccagttggatatagaagctacattccaccttgatgcctccgctgttgccgctttaaacgaagcggacgtcggttacaactgttcctcttctggtggtgctgtgaatagaggagctttgggtccattcggtttgttagttctcgcggctggagacagacgtggtgagcaaactgctgtttacttttatgttagtagaggtttggacggcggtttgcatacctccttctgtcaagatgaactcagaagttcccgcgcgaaggatgttactaaaagagtcatcggttcgactgtcccggttcttgacggcgaagcattctctatgagggttttagttgatcattcgattgtccaaggttttgcaatgggtggtagaactacgatgacatctcgggtctatccaatggaagcttaccaggaggccaaggtttacctctttaacaacgctaccggagcatccgttaccgctgaaagacttgtagttcacgatatggactcagcccataatcaattgtctaacatggacgactactcatatgtacagtaa

SEQ ID NO: 54 - DNA, 1935 bp, Artificial Sequence
  Synthetic
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgacgaagaggctgccggtggatttccctggtcaaacgaaatgttacaatggcagagatccggttaccacttccaaacagcaaaaaattatatgtctgatcctaacggcctaatgtactataggggttggaaccatatgttcttccaatacaatccagtcgggactgattgggacgacggtatggaatggggtcacgctgtgtcgcgtaacttggtacaatggagaacgttgccaatagctatggttgccgatcaatggtacgatattctgggtgttctttctggttctatgaccgtcttgccaaatggtactgttatcatgatctataccggtgctactaacgcgagcgctgtcgaagttcaatgtattgcaaccccagccgatccgacggaccctttgttaagaagatggactaagcatccagctaaccctgtgatctggagtccaccaggtgtagggacaaaggattttcgagatccaatgaccgcttggtacgacgaatcagacgatacttggagaacgctattgggctctaaggatgacaataatggtcaccacgacggtattgctatgatgtacaaaactaaggatttcttgaactacgagctgattcctggtatcctccatagagttgaaagaacaggagaatgggaatgcatagacttttatccggtcggtcgtagaacctctgataactcgtccgaaatgttgcatgttttaaaggcttccatggatgacgagagacacgactactactctctaggtacttatgatagtgccgccaataggtggactccaattgacccagaattggatttgggtattggtttgagatatgactgggggaaattctacgcttccaccagcttctatgatcccgcaaagaagagaagagttttgatgggttacgtcggtgaagtggactctaaacgcgctgacgttgttaagggttgggcctctatccaaagtgtcccacgcaccattgctctggacgaaaaaactcgtacaaaccttttattgtggccagtagaagaaatcgaaaccttaagattgaacgctactgagttgtccgacgttactttaaacactggttccgtcatccacattccattgagacagggaacccaattggatattgaagcaacctttcatctcgatgcgagtgctgttgcagctttcaatgaagctgatgtcggttacaattgttcatcttcgggtggtgctgttaatagaggtgctctagggcctttcggcctcttagtcttggctgccggtgatagaagaggtgaacaaaccgctgtttacttttacgtatctcgtggtttggacggcggtctacacacctctttttgtcaggatgagttaagatcctcaagggctaaggacgttactaagagagtcataggatcaactgtgcccgttttggatggtgaagccttttctatgcgtgtacttgttgatcattccatagtccaaggtttcgcaatgggtggtagaacaactatgacgagcagagtttatccaatggaagcgtaccaagaagctaaggtttatcttttcaacaacgcaacaggtgcctctgttacagccgagagattggtcgtacacgaaatggactccgcccacaaccaattgtcgaacatggacgaccactcgtatgttcaataa

SEQ ID NO: 55 - DNA, 1950 bp, Artificial Sequence
  Synthetic
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctagtggcccttattctgcttcgggtggttttccatggtctaatgccatgttgcagtggcaacgtacaggataccacttccaacccgaaaaaaactaccaaaacgacccaaacggtccagtctactataagggttggtatcatttcttttaccaacataatccaggtggtaccgggtggggtaacatctcatggggtcacgcagtttccagagatatggtacactggaggcatttaccactagctatggttcctgagcattggtacgatatagaaggtgttttgactggaagcattactgtccttccagacggtagagtcattttgttatataccggcaatactgaaacgttcgctcaagtgacctgtttggcggaggctgccgacccttccgatccactgttgagagaatgggctaagcacccggccaacccagtagtttacccgccaccaggtatcggtatgaaagactacagagatccaactacagcttggttcgataactcagacaatacctggagaataatcattggttctaagaatgatactgatcactctggtatcgtttttacttacaagaccaaggacttcgtcagctacgaactgattcctggatacctatatagaggtccagccgggacgggtatgtacgaatgcattgatttgttcgctgttggtggtgggcgtgctgcatcagatatgtataactctaccgctgaagatgtcttatacgttttgaaagaatcctccgacgacgacagacgggattactatgccttagggcgatttgacgctgccgctaatacttggacacccatagatacagaaagagagttgggtgtcgcactcagatatgattacggtagatacgatacttctaagtctttctacgacccagttaagcaaaggagaattgtctggggttacgttgtcgaaaccgacagttggtccgctgacgctgcaaaaggttgggctaacctgcaatctatccctagaactgttgaattggatgaaaagactcgaacaaaccttgtacagtggccagtgggtgagttgaacaccctacgtatcaataccactgatttgagtgacattaccgttggtgctggctcggtcgattctttacccttgcaccaaacttcccaactagacatcgaagcgtcatttagaattaatgcctctactatagaagccttgaacgaagttgatgtaggttataactgtactatgacgtctggtgctgctactagaggtgctttgggtccattcggaattttagtcttggctaacgtggccttgacagaacagaccgctgtttatttttatgtttccaagggtttagacggtggtttacgaacccacttctgtcatgacgaattgaggtctacacacgctaccgacgtcgccaaggaggttgttgggtctactgttccagttctcgatggtgaagattttagcgtcagagttttggtcgatcactcaatcgtacaatctttcgtcatgggtggcagaatgacagcaacttccagagcttacccgactgaagcaatctatgctgccgctggcgtttacctcttcaacaatgctacaggtgcttccattaccgcagaaaaattggtggtacatgacatggattcctcctacaacagaatctttactgacgaggatttattggtgcttgactaa

SEQ ID NO: 56 - DNA, 1935 bp, Artificial Sequence
  Synthetic
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgcaaatgcttttccttggtcgaacgctatgttgcagtggcaacgtactggcttccatttccaaccagacaaatactatcaaaacgatccaaacggtcccgtctactacggaggttggtatcactttttctaccaatataatccgtctggtagtgtttgggagccacaaattgtatggggtcacgccgtttccaaggacctgatccattggcggcacttaccaccagctttggtcccagatcaatggtacgacataaagggtgttctaaccgggtcaattacggtccttcctgatggtaaggtgatcttgttatatactggtaatacagaaaccttcgctcaagttacttgcttggccgaacccgcagatccaagcgatccattgctcagagaatgggtaaagcatcctgctaacccagttgtctttccaccacccggtattggtatgaaagacttcagagatccaaccactgcttggtacgacgaatctgacggcacatggagaaccatcattggatctaaaaacgactccgaccactctggtatcgttttttcctacaagactaaggatttcattagttatgagttgatgccgggttacatgtacagaggcccaaaggggaccggtgaatacgaatgtatagatttatacgcggtgggtggtggtaggaaggcttctgatatgtataactccactgcggaagatgtcctatatgttttaaaagaatcatctgacgatgatagacatgactggtactcattgggtagatttgacgccgctgctaataagtggacacctatagatactgagcttgaacttggcgttggtttgcgatatgactggggtaagtactacgccagcaagtctttctacgacccagttaaaaaaagacgtgtcgtgtgggcttatgtcggtgaaaccgattccgaaagagccgacatcaccaagggttgggcaaatttgcagtctatcccacgcactgttgaattggacgaaaaaactagaacgaacttaattcaatggccggttgaggaactaaatacactgcgtattaacactacagatttgtcgggaatcaccgtaggtgctggtagtgtcgctttcttgccattgcaccaaactgcccagctcgacattgaagctacttttagaattgatgcttctgcgatagaagctctaaacgaagctgatgtttcctacaattgtaccacatcgcgaggagctgctaccagaggtgccttaggtccattcggtttgttggtattagccaaccatgccttgaccgaacaaactggtgtttacttttacgtgtctaagggtttggacggtggtttaagaactcacttctgtcacgatgaactaagatcctctcatgcttcagatgtcgttaagagagtcgtgggtagtacggttcctgttttggatggggaggactttagcgttcgtgtcttggttgaccactctattgtccaaagtttcgccatgggtggtaggttgacagctacctccagagcttatccaactgaagcaatctacgctgcggcaggcgtatacatgttcaacaacgctacaggtacttccgttacggctgaaaagcttgttgtccacgatatggattcttcctacaaccacatctataccgacggtgacctggtggtagttgattaa

SEQ ID NO: 57 - DNA, 1953 bp, Artificial Sequence
  Synthetic
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgacgatcctccatctgatagtgaagattacccatggaccaatgagatgcttaaatggcaaaggacgggttatcacttccagcccccaaaccattttatggcagacccaaacgccgctatgtactacaaggggtggtatcacttcttttaccaatataaccctaatggttcagcttgggactactccatctcgtggggtcatgctgtatctaaggacatgattcactggctgcatttaccagtcgccatggttccagatcattggtacgatagcaaaggagtttggtccggctacgctactactttgccagatggtagaataattgtcttgtataccggtggtacagaccaattggttcaagtgcaaaatttagccgaaccagcggacccttctgatccactattgatcgaatggaagaagtcaaacggaaacccaattttgatgcctccgccgggtgtaggtccacacgatttcagagatccattcccagtttggtacaacgaatctgactccacatggcacatgttgatcggttctaaagatgacaatcactacggtaccgttctaatttatactactaaggattttgagacatacactttattgccagacatcctacataagaccaaggactcggttggtatgttggaatgtgtcgatctttatccagtggctactaccgggaatcaaattggtaacggtttagaaatgaaaggtggttccggcaagggtatcaagcacgtcctgaaggcttctatggacgatgaacgtcacgattattacgccataggtacgttcgacttggaatcctttagttgggttccggacgacgataccatagatgtcggcgtcggcttgcgctatgactacggtaagttctacgcttcaaaaactttctatgatcaggaaaagaagagaagaattttgtggggatacgttggtgaagtagactctaaggctgacgacatcttaaaaggttgggcgagcgttcaaaatattgcaagaactatcctatttgatgcaaaaactagaagtaacttgctcgtctggcccgtcgaggaattggacgctttgcgaacctctggtaaggaatttaacggtgtggttgttgaacctggttctacttaccatttagacgtaggtaccgccacccaattggatattgaagctgaatttgagatcaataaggaagctgttgacgctgttgtcgaagccgatgttacatacaactgctccacatctgatggtgctgctcacagaggtttgttgggaccattcggtcttttggttttagctaatgaaaagatgacagaaaaaaccgccacttatttctacgtcagtcgtaacgttgatgggggtctacaaactcatttctgtcaagacgagcttagaagctctaaagctaacgatattaccaaacgtgtcgttggccacactgttccagttctgcatggtgaaaccttctccttgagaattttagtagaccactcgatcgttgaatcgtttgcgcagaagggtagagcagtcgctacgtctagggtgtatccaactgaagctatctacgattctacaagagttttcctcttcaacaacgccacttcagctacggtcactgccaagtccgtaaagatatggcatatgaacagtacccataaccacccttttccaggtttccccgcaccataa

SEQ ID NO: 58 - DNA, 1956 bp, Artificial Sequence
  Synthetic
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctaacttgatgcgtttaagagagaatgattatccctggactaacgacatgctaagatggcaacgcacgggatttcacttccagcctgaaaaaaacttccaagccgacccaaatgcagctatgttttacaagggctggtaccatttcttttatcaatacaacccgaccggtgtggcttgggattacacaatctcctggggtcacgctgtcagtaaggatttgctgcattggaattatcttccaatggccttgaggcctgaccactggtacgatagaaaaggtgtttggagcggttactctactttattgccagacggtagaattgttgtcttgtacaccggtggaactaaggaattagttcaagtccaaaacttggctgtcccagtaaacctttctgacccattgctattggaatggaagaagtcacacgttaacccaatactcgttccacctccggggatcgaggatcatgatttccgagatccattcccagtgtggtataatgaatctgactcgcggtggcacgttgtaattggttccaaagatccagaacactatggtattgtcttgatctacactaccaaggacttcgttaactttacgttattaccaaacatattgcattccaccaagcagccggttggtatgctggaatgtgtagacttgttcccagttgctacaactgattctcgtgcaaatcaagctttggatatgactaccatgaggcccggtcctggcctcaaatatgtgttaaaggcgagtatggatgacgaaagacacgattactacgccctaggtagctttgacttggactcgttcacttttacaccagatgatgaaaccattgacgtcggtgtcggtttgagatacgactggggtaagttctatgcttcaaaaactttctatgaccaagaaaagcatagaagagttttatggggttacgtgggggaagttgattctaagagagatgacgcgttaaaaggctgggcttccttgcaaaacatcccaagaacaattttgttcgataccaaaactaagtctaatctaatcttgtggccagttgaagaggtcgaatcattgagaactattaacaagaattttaactctataccactttacccaggttccacttaccaattggatgttggggaagccacccaactggatattgtcgctgaatttgaagtcgatgagaaggctattgaagcaactgctgaagctgacgttacatataactgctctaccagcggtggtgccgctaacagaggtgttttgggtcctttcggtctattggttctagccaatcaagaactttccgaacagactgccacttacttctatgtatcgcgtggtatcgacggcaacctgagaacccacttttgtcaagacgaattgagatcctccaaagccggtgctatcaccaagagggtcgtaggttctacagttcctgttttgcatggtgaaacgtgggctttacgtatcctagttgaccactctattgtcgagtcttttgcacaacggggacgcgccgtcgctaccagtagagtatacccaactgaggctatatactcttcggctagagtctttctcttcaataacgcaaccgatgccattgttacagctaaaacggtcaacgtttggcatatgaatagcacttacaaccacgtctttcctggtttggttgctccataa

SEQ ID NO: 59 - DNA, 1953 bp, Artificial Sequence
  Synthetic
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgacgatcctccatctgatagtgaagattacccatggaccaatgagatgcttaaatggcaaaggacgggttatcacttccagcccccaaaccattttatggcagacccaaacgccgctatgtactacaaggggtggtatcacttcttttaccaatataaccctaatggttcagcttgggactactccatctcgtggggtcatgctgtatctaaggacatgattcactggctgcatttaccagtcgccatggttccagatcattggtacgatagcaaaggagtttggtccggctacgctactactttgccagatggtagaataattgtcttgtataccggtggtacagaccaattggttcaagtgcaaaatttagccgaaccagcggacccttctgatccactattgatcgaatggaagaagtcaaacggaaacccaattttgatgcctccgccgggtgtaggtccacacgatttcagagatccattcccagtttggtacaacgaatctgactccacatggcacatgttgatcggttctaaagatgacaatcactacggtaccgttctaatttatactactaaggattttgagacatacactttattgccagacatcctacataagaccaaggactcggttggtatgttggaatgtgtcgatctttatccagtggctactaccgggaatcaaattggtaacggtttagaaatgaaaggtggttccggcaagggtatcaagcacgtcctgaaggcttctatggacgatgaacgtcacgattattacgccataggtacgttcgacttggaatcctttagttgggttccggacgacgataccatagatgtcggcgtcggcttgcgctatgactacggtaagttctacgcttcaaaaactttctatgatcaggaaaagaagagaagaattttgtggggatacgttggtgaagtagactctaaggctgacgacatcttaaaaggttgggcgagcgttcaaaatattgcaagaactatcctatttgatgcaaaaactagaagtaacttgctcgtctggcccgtcgaggaattggacgctttgcgaacctctggtaaggaatttaacggtgtggttgttgaacctggttctacttaccatttagacgtaggtaccgccacccaattggatattgaagctgaatttgagatcaataaggaagctgttgacgctgttgtcgaagccgatgttacatacaactgctccacatctgatggtgctgctcacagaggtttgttgggaccattcggtcttttggttttagctaatgaaaagatgacagaaaaaaccgccacttatttctacgtcagtcgtaacgctgatgggggtctacaaactcatttctgtcaagacgagcttagaagctctaaagctaacgatattaccaaacgtgtcgttggccacactgttccagttctgcatggtgaaaccttctccttgagaattttagtcgatcactcaattgtcgagtccttcgcgcaaaagggtagggctgttgcaacctctcgggtgtatccaactgaagccatctacgattctacgagagtttttctcttcaacaacgctacttcggcaacggtaactgctaagtccgtaaagatatggcatatgaacagtacccataaccacccttttccaggtttccccgcgccataa

SEQ ID NO: 60 - protein, 89 aa, Artificial Sequence
  Synthetic
MRFPSIFTAVLFAASSALAAPVNTTTEDETAQIPAEAVIGYSDLEGDFDVAVLPFSNSTNNGLLFINTTIASIAAKEEGVSLEKREAEA

SEQ ID NO: 61 - DNA, 267 bp, Artificial Sequence
  Synthetic
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagct

SEQ ID NO: 62 - DNA, 1956 bp, Artificial Sequence
  Synthetic
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctaacttgatgcgtttaagagagaatgattatccctggactaacgacatgctaagatggcaacgcacgggatttcacttccagcctggtaaaaacttccaagccgacccaaatgcagctatgttttacaagggctggtaccatttcttttatcaatacaacccgaccggtgtggcttgggattacacaatctcctggggtcacgctgtcagtaaggatttgctgcattggaattatcttccaatggccttgaggcctgaccactggtacgatagaaaaggtgtttggagcggttactctactttattgccagacggtagaattgttgtcttgtacaccggtggaactaaggaattagttcaagtccaaaacttggctgtcccagtaaacctttctgacccattgctattggaatggaagaagtcacacgttaacccaatactcgttccacctccggggatcgaagatcatgatttccgagatccattcccagtgtggtataatgaatctgactcgcggtggcacgttgtaattggttccaaagatccagagcactatggtattgtcttgatctacactaccaaggacttcgttaactttacgttattaccaaacatattgcattccaccaagcagccggttggtatgctggaatgtgtagacttgttcccagttgctacaactgattctcgtgcaaatcaagctttggatatgactaccatgaggcccggtcctgggctcaaatatgtgttaaaggcgagtatggatgacgaaagacacgattactacgccctaggtagctttgacttggactcgttcacttttacaccagatgatgaaaccattgacgtcggtattggtcttagatacgactggggcaagttctacgcgtccaagactttttacgaccaagaaaaacaaagaagagttttgtggggatacgtcggtgaagttgactcgaagcgtgatgatgctctgaaaggttgggcttctttgcaaaatatcccacgtacaatcttgttcgacaccaaaaccaagtccaacctaattttgtggccagttgaagaagtcgagtctttaagaactattaacaagaatttcaattcaatccctttgtatcctggttctacttaccagcttgatgtgggtgaagctacccaattggatattgtggccgagttcgaagtcgatgaaaaggctattgaagctactgccgaagctgatgttacatataactgctccacctccggtggtgcagctaatagaggggttttgggtccattcggtttgttagttttagctaaccaagagttgtctgaacaaactgctacttacttctatgtctctcgcggcatagatggtaacttaagaacacatttttgtcaagacgaactgcgatcttccaaggctggtgccatcactaagcgggtagttggttctaccgtcccagttctacatggcgaaacctgggccttgagaattttggtcgatcactcaatcgtagagtcttttgcacagagaggtagagctgttgccacgagtagagtctatcctacagaagcaatttatagctcagctagagtctttctattcaacaatgccactgacgctattgttaccgctaagacagtaaacgtttggcacatcaactccacctacaatcatgtttttccgggtctggtcgctccataa

SEQ ID NO: 63 - protein, 564 aa, Artificial Sequence
  Synthetic
GARVGLGGIYDDADAFAWNNSMLQWQRAGFHFQTEKNFMSDPNGPVYYRGYYHLFYQYNMKGVVWDDGIVWGHVVSRDLVHWRHLPIAMVPDHWYDSMGVLSGSITVLQNGSLVMIYTGVFSKTTDRSGMMEVQCLAVPADPNDPLLRSWTKHPANPVLVHPPGIKDMDFRDPTTAWFDESDSTYRTVIGTKDDHHGSHAGFAMVYKTKDFLSFQRIPGILHSVEHTGMWECMDFYPVGGGDNSSSEVLYVIKASMDDERHDYYALGMYDAAANTWTPLDQELDLGIGLRYDWGKLYASTTFYDPAKRRRVMLGYVGETDSRRSDEAKGWASIQSIPRTVALDEKTRTNLLLWPVEEIETLRLNATEFNDINIDTGSVFHLPIRQGNQLDIEASFRLDASAVAAINEADVGYNCSSSGGAATRGALGPFGLLVLAAEGIGEQTAVYFYVSRGLDGGLRTSFCNDELRSSWARDVTKRVVGSTVPVLNGETLSMRVLVDHSIVQSFAMGGRVTATSRVYPTEAIYAAAGVYLFNNATNASVTAERIIVHEMDSIDNNQIFLIDDL